SEQUENCE LISTING (113 sequences)

SEQ ID NO: 1 - protein, 178 aa, Artificial Sequence
  Caveolin-1
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 2 - protein, 20 aa, Artificial Sequence
  Caveolin-1 scaffolding domain (CSD)
DGIWKASFTTFTVTKYWFYR

SEQ ID NO: 3 - protein, 7 aa, Artificial Sequence
  CSP-7
FTTFTVT

SEQ ID NO: 4 - protein, 19 aa, Artificial Sequence
  Var55
  MOD_RES (1)..(1): ACETYLATION
  SITE (1)..(2): D-Ala
  SITE (18)..(19): D-Ala
  MOD_RES (19)..(19): AMIDATION
AAEGKASFTTFTVTKGSAA

SEQ ID NO: 5 - protein, 13 aa, Artificial Sequence
  Caveolin-1 derivative
KASFTTFTVTKGS

SEQ ID NO: 6 - protein, 19 aa, Artificial Sequence
  Caveolin-1 derivative
  SITE (1)..(2): D-Ala
  SITE (18)..(19): D-Ala
AAEGKASFTTFTVTKGSAA

SEQ ID NO: 7 - protein, 12 aa, Artificial Sequence
  Caveolin-1 derivative
  MOD_RES (1)..(1): Orn
  MOD_RES (11)..(11): Orn
XASFTTFTVTXS

SEQ ID NO: 8 - protein, 19 aa, Artificial Sequence
  Caveolin-1 derivative
  SITE (1)..(2): D-Ala
  SITE (18)..(19): D-Ala
  MOD_RES (19)..(19): AMIDATION
AAEGKASFTTFTVTKGSAA

SEQ ID NO: 9 - protein, 9 aa, Artificial Sequence
  Caveolin-1 derivative
ASFTTFTVT

SEQ ID NO: 10 - protein, 12 aa, Artificial Sequence
  Caveolin-1 derivative
  MOD_RES (1)..(1): Orn
  MOD_RES (11)..(11): Orn
  MOD_RES (12)..(12): AMIDATION
XASFTTFTVTXS

SEQ ID NO: 11 - protein, 7 aa, Artificial Sequence
  Caveolin-1 derivative
  MOD_RES (7)..(7): AMIDATION
FTTFTVT

SEQ ID NO: 12 - protein, 8 aa, Artificial Sequence
  Caveolin-1 derivative
  MOD_RES (8)..(8): AMIDATION
FTTFTVTK

SEQ ID NO: 13 - protein, 11 aa, Artificial Sequence
  Caveolin-1 derivative
  MOD_RES (11)..(11): AMIDATION
KASFTTFTVTK

SEQ ID NO: 14 - protein, 11 aa, Artificial Sequence
  Caveolin-1 derivative
  MOD_RES (1)..(1): ACETYLATION
  MOD_RES (11)..(11): AMIDATION
KASFTTFTVTK

SEQ ID NO: 15 - protein, 11 aa, Artificial Sequence
  Caveolin-1 derivative
  MOD_RES (1)..(1): Orn
  MOD_RES (11)..(11): AMIDATION
XASFTTFTVTK

SEQ ID NO: 16 - protein, 11 aa, Artificial Sequence
  Caveolin-1 derivative
  MOD_RES (1)..(1): Orn
  MOD_RES (1)..(1): ACETYLATION
  MOD_RES (11)..(11): AMIDATION
XASFTTFTVTK

SEQ ID NO: 17 - protein, 13 aa, Artificial Sequence
  Caveolin-1 derivative
  MOD_RES (1)..(1): ACETYLATION
  MOD_RES (13)..(13): AMIDATION
KASFTTFTVTKGS

SEQ ID NO: 18 - protein, 14 aa, Artificial Sequence
  Caveolin-1 derivative
  MOD_RES (14)..(14): AMIDATION
DSGKASFTTFTVTK

SEQ ID NO: 19 - protein, 14 aa, Artificial Sequence
  Caveolin-1 derivative
  MOD_RES (1)..(1): ACETYLATION
  MOD_RES (14)..(14): AMIDATION
DSGKASFTTFTVTK

SEQ ID NO: 20 - protein, 12 aa, Artificial Sequence
  Caveolin-1 derivative
  MOD_RES (1)..(1): Orn
  MOD_RES (1)..(1): ACETYLATION
  MOD_RES (11)..(11): Orn
  MOD_RES (12)..(12): AMIDATION
XASFTTFTVTXS

SEQ ID NO: 21 - protein, 13 aa, Artificial Sequence
  Caveolin-1 derivative
  MOD_RES (13)..(13): AMIDATION
KASFTTFTVTKGS

SEQ ID NO: 22 - protein, 12 aa, Artificial Sequence
  Caveolin-1 derivative
KASFTTFTVTKY

SEQ ID NO: 23 - protein, 12 aa, Artificial Sequence
  Caveolin-1 derivative
IWKASFTTFTVT

SEQ ID NO: 24 - protein, 13 aa, Artificial Sequence
  Caveolin-1 derivative
SFTTFTVTKYWFY

SEQ ID NO: 25 - protein, 13 aa, Artificial Sequence
  Caveolin-1 derivative
KASFTTFTVTKYW

SEQ ID NO: 26 - protein, 13 aa, Artificial Sequence
  Caveolin-1 derivative
IWKASFTTFTVTK

SEQ ID NO: 27 - protein, 14 aa, Artificial Sequence
  Caveolin-1 derivative
FTTFTVTKYWFYRL

SEQ ID NO: 28 - protein, 14 aa, Artificial Sequence
  Caveolin-1 derivative
ASFTTFTVTKYWFY

SEQ ID NO: 29 - protein, 14 aa, Artificial Sequence
  Caveolin-1 derivative
WKASFTTFTVTKYW

SEQ ID NO: 30 - protein, 14 aa, Artificial Sequence
  Caveolin-1 derivative
GIWKASFTTFTVTK

SEQ ID NO: 31 - protein, 15 aa, Artificial Sequence
  Caveolin-1 derivative
FTTFTVTKYWFYRLL

SEQ ID NO: 32 - protein, 15 aa, Artificial Sequence
  Caveolin-1 derivative
ASFTTFTVTKYWFYR

SEQ ID NO: 33 - protein, 15 aa, Artificial Sequence
  Caveolin-1 derivative
WKASFTTFTVTKYWF

SEQ ID NO: 34 - protein, 15 aa, Artificial Sequence
  Caveolin-1 derivative
GIWKASFTTFTVTKY

SEQ ID NO: 35 - protein, 15 aa, Artificial Sequence
  Caveolin-1 derivative
FDGIWKASFTTFTVT

SEQ ID NO: 36 - protein, 16 aa, Artificial Sequence
  Caveolin-1 derivative
SFTTFTVTKYWFYRLL

SEQ ID NO: 37 - protein, 16 aa, Artificial Sequence
  Caveolin-1 derivative
KASFTTFTVTKYWFYR

SEQ ID NO: 38 - protein, 16 aa, Artificial Sequence
  Caveolin-1 derivative
IWKASFTTFTVTKYWF

SEQ ID NO: 39 - protein, 16 aa, Artificial Sequence
  Caveolin-1 derivative
DGIWKASFTTFTVTKY

SEQ ID NO: 40 - protein, 16 aa, Artificial Sequence
  Caveolin-1 derivative
SFDGIWKASFTTFTVT

SEQ ID NO: 41 - protein, 17 aa, Artificial Sequence
  Caveolin-1 derivative
SFTTFTVTKYWFYRLLS

SEQ ID NO: 42 - protein, 17 aa, Artificial Sequence
  Caveolin-1 derivative
KASFTTFTVTKYWFYRL

SEQ ID NO: 43 - protein, 17 aa, Artificial Sequence
  Caveolin-1 derivative
IWKASFTTFTVTKYWFY

SEQ ID NO: 44 - protein, 17 aa, Artificial Sequence
  Caveolin-1 derivative
DGIWKASFTTFTVTKYW

SEQ ID NO: 45 - protein, 17 aa, Artificial Sequence
  Caveolin-1 derivative
SFDGIWKASFTTFTVTK

SEQ ID NO: 46 - protein, 18 aa, Artificial Sequence
  Caveolin-1 derivative
FTTFTVTKYWFYRLLSAL

SEQ ID NO: 47 - protein, 18 aa, Artificial Sequence
  Caveolin-1 derivative
ASFTTFTVTKYWFYRLLS

SEQ ID NO: 48 - protein, 18 aa, Artificial Sequence
  Caveolin-1 derivative
WKASFTTFTVTKYWFYRL

SEQ ID NO: 49 - protein, 18 aa, Artificial Sequence
  Caveolin-1 derivative
GIWKASFTTFTVTKYWFY

SEQ ID NO: 50 - protein, 18 aa, Artificial Sequence
  Caveolin-1 derivative
FDGIWKASFTTFTVTKYW

SEQ ID NO: 51 - protein, 18 aa, Artificial Sequence
  Caveolin-1 derivative
HSFDGIWKASFTTFTVTK

SEQ ID NO: 52 - protein, 19 aa, Artificial Sequence
  Caveolin-1 derivative
FTTFTVTKYWFYRLLSALF

SEQ ID NO: 53 - protein, 19 aa, Artificial Sequence
  Caveolin-1 derivative
ASFTTFTVTKYWFYRLLSA

SEQ ID NO: 54 - protein, 19 aa, Artificial Sequence
  Caveolin-1 derivative
WKASFTTFTVTKYWFYRLL

SEQ ID NO: 55 - protein, 19 aa, Artificial Sequence
  Caveolin-1 derivative
GIWKASFTTFTVTKYWFYR

SEQ ID NO: 56 - protein, 19 aa, Artificial Sequence
  Caveolin-1 derivative
FDGIWKASFTTFTVTKYWF

SEQ ID NO: 57 - protein, 19 aa, Artificial Sequence
  Caveolin-1 derivative
HSFDGIWKASFTTFTVTKY

SEQ ID NO: 58 - protein, 19 aa, Artificial Sequence
  Caveolin-1 derivative
GTHSFDGIWKASFTTFTVT

SEQ ID NO: 59 - protein, 8 aa, Artificial Sequence
  Caveolin-1 derivative
FTTFTVTK

SEQ ID NO: 60 - protein, 8 aa, Artificial Sequence
  Caveolin-1 derivative
SFTTFTVT

SEQ ID NO: 61 - protein, 9 aa, Artificial Sequence
  Caveolin-1 derivative
FTTFTVTKY

SEQ ID NO: 62 - protein, 9 aa, Artificial Sequence
  Caveolin-1 derivative
SFTTFTVTK

SEQ ID NO: 63 - protein, 10 aa, Artificial Sequence
  Caveolin-1 derivative
FTTFTVTKYW

SEQ ID NO: 64 - protein, 10 aa, Artificial Sequence
  Caveolin-1 derivative
SFTTFTVTKY

SEQ ID NO: 65 - protein, 10 aa, Artificial Sequence
  Caveolin-1 derivative
ASFTTFTVTK

SEQ ID NO: 66 - protein, 10 aa, Artificial Sequence
  Caveolin-1 derivative
KASFTTFTVT

SEQ ID NO: 67 - protein, 11 aa, Artificial Sequence
  Caveolin-1 derivative
FTTFTVTKYWF

SEQ ID NO: 68 - protein, 11 aa, Artificial Sequence
  Caveolin-1 derivative
SFTTFTVTKYW

SEQ ID NO: 69 - protein, 11 aa, Artificial Sequence
  Caveolin-1 derivative
ASFTTFTVTKY

SEQ ID NO: 70 - protein, 11 aa, Artificial Sequence
  Caveolin-1 derivative
KASFTTFTVTK

SEQ ID NO: 71 - protein, 11 aa, Artificial Sequence
  Caveolin-1 derivative
WKASFTTFTVT

SEQ ID NO: 72 - protein, 12 aa, Artificial Sequence
  Caveolin-1 derivative
FTTFTVTKYWFY

SEQ ID NO: 73 - protein, 12 aa, Artificial Sequence
  Caveolin-1 derivative
SFTTFTVTKYWF

SEQ ID NO: 74 - protein, 12 aa, Artificial Sequence
  Caveolin-1 derivative
ASFTTFTVTKYW

SEQ ID NO: 75 - protein, 12 aa, Artificial Sequence
  Caveolin-1 derivative
WKASFTTFTVTK

SEQ ID NO: 76 - protein, 13 aa, Artificial Sequence
  Caveolin-1 derivative
FTTFTVTKYWFYR

SEQ ID NO: 77 - protein, 13 aa, Artificial Sequence
  Caveolin-1 derivative
ASFTTFTVTKYWF

SEQ ID NO: 78 - protein, 13 aa, Artificial Sequence
  Caveolin-1 derivative
WKASFTTFTVTKY

SEQ ID NO: 79 - protein, 13 aa, Artificial Sequence
  Caveolin-1 derivative
GIWKASFTTFTVT

SEQ ID NO: 80 - protein, 14 aa, Artificial Sequence
  Caveolin-1 derivative
SFTTFTVTKYWFYR

SEQ ID NO: 81 - protein, 14 aa, Artificial Sequence
  Caveolin-1 derivative
KASFTTFTVTKYWF

SEQ ID NO: 82 - protein, 14 aa, Artificial Sequence
  Caveolin-1 derivative
IWKASFTTFTVTKY

SEQ ID NO: 83 - protein, 14 aa, Artificial Sequence
  Caveolin-1 derivative
DGIWKASFTTFTVT

SEQ ID NO: 84 - protein, 15 aa, Artificial Sequence
  Caveolin-1 derivative
SFTTFTVTKYWFYRL

SEQ ID NO: 85 - protein, 15 aa, Artificial Sequence
  Caveolin-1 derivative
KASFTTFTVTKYWFY

SEQ ID NO: 86 - protein, 15 aa, Artificial Sequence
  Caveolin-1 derivative
IWKASFTTFTVTKYW

SEQ ID NO: 87 - protein, 15 aa, Artificial Sequence
  Caveolin-1 derivative
DGIWKASFTTFTVTK

SEQ ID NO: 88 - protein, 16 aa, Artificial Sequence
  Caveolin-1 derivative
FTTFTVTKYWFYRLLS

SEQ ID NO: 89 - protein, 16 aa, Artificial Sequence
  Caveolin-1 derivative
ASFTTFTVTKYWFYRL

SEQ ID NO: 90 - protein, 16 aa, Artificial Sequence
  Caveolin-1 derivative
WKASFTTFTVTKYWFY

SEQ ID NO: 91 - protein, 16 aa, Artificial Sequence
  Caveolin-1 derivative
GIWKASFTTFTVTKYW

SEQ ID NO: 92 - protein, 16 aa, Artificial Sequence
  Caveolin-1 derivative
FDGIWKASFTTFTVTK

SEQ ID NO: 93 - protein, 17 aa, Artificial Sequence
  Caveolin-1 derivative
FTTFTVTKYWFYRLLSA

SEQ ID NO: 94 - protein, 17 aa, Artificial Sequence
  Caveolin-1 derivative
ASFTTFTVTKYWFYRLL

SEQ ID NO: 95 - protein, 17 aa, Artificial Sequence
  Caveolin-1 derivative
WKASFTTFTVTKYWFYR

SEQ ID NO: 96 - protein, 17 aa, Artificial Sequence
  Caveolin-1 derivative
GIWKASFTTFTVTKYWF

SEQ ID NO: 97 - protein, 17 aa, Artificial Sequence
  Caveolin-1 derivative
FDGIWKASFTTFTVTKY

SEQ ID NO: 98 - protein, 17 aa, Artificial Sequence
  Caveolin-1 derivative
HSFDGIWKASFTTFTVT

SEQ ID NO: 99 - protein, 18 aa, Artificial Sequence
  Caveolin-1 derivative
SFTTFTVTKYWFYRLLSA

SEQ ID NO: 100 - protein, 18 aa, Artificial Sequence
  Caveolin-1 derivative
KASFTTFTVTKYWFYRLL

SEQ ID NO: 101 - protein, 18 aa, Artificial Sequence
  Caveolin-1 derivative
IWKASFTTFTVTKYWFYR

SEQ ID NO: 102 - protein, 18 aa, Artificial Sequence
  Caveolin-1 derivative
DGIWKASFTTFTVTKYWF

SEQ ID NO: 103 - protein, 18 aa, Artificial Sequence
  Caveolin-1 derivative
SFDGIWKASFTTFTVTKY

SEQ ID NO: 104 - protein, 18 aa, Artificial Sequence
  Caveolin-1 derivative
THSFDGIWKASFTTFTVT

SEQ ID NO: 105 - protein, 19 aa, Artificial Sequence
  Caveolin-1 derivative
SFTTFTVTKYWFYRLLSAL

SEQ ID NO: 106 - protein, 19 aa, Artificial Sequence
  Caveolin-1 derivative
KASFTTFTVTKYWFYRLLS

SEQ ID NO: 107 - protein, 19 aa, Artificial Sequence
  Caveolin-1 derivative
IWKASFTTFTVTKYWFYRL

SEQ ID NO: 108 - protein, 19 aa, Artificial Sequence
  Caveolin-1 derivative
DGIWKASFTTFTVTKYWFY

SEQ ID NO: 109 - protein, 19 aa, Artificial Sequence
  Caveolin-1 derivative
SFDGIWKASFTTFTVTKYW

SEQ ID NO: 110 - protein, 19 aa, Artificial Sequence
  Caveolin-1 derivative
THSFDGIWKASFTTFTVTK

SEQ ID NO: 111 - protein, 13 aa, Artificial Sequence
  Cell penetrating peptide
GRKKRRQRRRPPQ

SEQ ID NO: 112 - protein, 16 aa, Artificial Sequence
  Cell penetrating peptide
RQIKIWFQNRRMKWKK

SEQ ID NO: 113 - protein, 26 aa, Artificial Sequence
  Cell penetrating peptide
GIGAVLKVLTTGLPALISWIKRKRQQ

(19) United States
(12) Patent Application Publication
(10) Pub. No.: US 2025/0076312 A1
(43) Pub. Date: Mar. 6, 2025

(54) BIOMARKERS FOR THE TREATMENT OF INTERSTITIAL LUNG DISEASE

(71) Applicant: Lung Therapeutics, LLC, Austin, TX (US)

(72) Inventors: BreAnne MACKENZIE, Austin, TX (US); Cory HOGABOAM, Los Angeles, CA (US)

(73) Assignee: Lung Therapeutics, LLC, Austin, TX (US)

(21) Appl. No.: 18/547,931

(22) PCT Filed: Feb. 25, 2022
(86) PCT No.: PCT/US2022/017833

§ 371 Date: Aug. 25, 2023

Related U.S. Application Data
(60) Provisional application No. 63/153,565, filed on Feb. 25, 2021.
(60) Provisional application No. 63/270,867, filed on Oct. 22, 2021.

(51) Int. Cl. G01N 33/68; A61K 38/17

(52) U.S. Cl. CPC G01N 33/6884; A61K 38/177; G01N 2333/521; G01N 2333/5431; G01N 2333/705; G01N 2333/70503; G01N 2333/70596; G01N 2333/71; G01N 2333/96494; G01N 2800/12; G01N 2800/52

(57) ABSTRACT

The present disclosure provides biomarkers associated with caveolin-1 peptide therapy in subjects with interstitial lung disease. In particular, the present disclosure describes biomarkers such as MYDGF, soluble RAGE, pSMAD2/3, and PDGFRβ associated with caveolin-1 peptide therapy in subjects with idiopathic pulmonary fibrosis. These biomarkers could be used to determine efficacy, monitoring, and optimal dosing of caveolin-1 peptide therapy in subjects with interstitial lung disease.

### CROSS-REFERENCE TO RELATED APPLICATIONS

This application claims the benefit of priority to U.S. Provisional Application No. 63/153,565, filed on Feb. 25, 2021, and U.S. Provisional Application No. 63/270,867, filed on Oct. 22, 2021, hereby incorporated by reference in their entireties.

### TECHNICAL FIELD

The present disclosure generally relates to the fields of medicine, pulmonary disease, and protein biology. In particular, the present disclosure relates to biomarkers associated with caveolin-1 peptide therapy for the treatment of interstitial lung disease, such as idiopathic pulmonary fibrosis.

### SEQUENCE LISTING

The Sequence Listing associated with this application is provided in text format in lieu of a paper copy, and is hereby incorporated by reference into the specification. The name of the text file containing the Sequence Listing is LUTX_020_02WO_SeqList_ST25.txt. The text file is about 31 KB in size, was created on Feb. 25, 2022, and is being submitted electronically via EFS-Web.

### BACKGROUND

Interstitial lung disease (ILD) is a group of over 200 respiratory diseases that cause fibrosis and inflammation of the tissue and space around the air sacs of the lungs. Idiopathic pulmonary fibrosis (IPF) is a type of ILD that is estimated to affect over 200,000 people in the United States and 1 out of every 200 adults over the age of 60 years old. IPF is characterized by progressive interstitial fibrosis of the lung parenchyma, and this interstitial fibrosis leads to loss of lung function, resulting in death due to respiratory failure in most patients. The median survival from the time of diagnosis is 2-3 years (Raghu et al., Am J Respir Crit Care Med 183:788-824 (2011)). The etiology and key molecular and pathophysiological drivers of IPF are unknown.

Pirfenidone and nintedanib are approved by the FDA for the treatment of IPF in the United States. Pirfenidone abrogates transforming growth factor beta 1 (TGF-β1)-stimulated collagen synthesis and down-regulates proinflammatory cytokines, and nintedanib is a broad spectrum tyrosine kinase inhibitor with anti-fibrotic properties. Although both compounds have been shown to slow disease progression in patients with IPF, neither treatment is known to be curative, and many patients experience side effects that reduce their quality of life. While lung transplantation is an option for IPF, it is expensive and associated with considerable morbidity, as only approximately 50% of lung-transplant recipients survive up to 5 years.

Caveolin-1 is an integral membrane protein that has homeostatic function in the fibrosis process by participating in a series of key regulatory pathways, such as TGF-β signaling (Shihata et al., Front. Pharmacol. 2017; 8:567; and Gvaramia et al., Matrix Biol, 2013: 32 (6): 307-315). Endogenous caveolin-1 was constitutively suppressed in fibrotic lungs in a bleomycin-induced animal model of IPF and IPF patients (Wang et al., J Exp Med, 2006; 203 (13): 2895-2906; Sanders et al., PLOS One, 2015; 10 (2), e0116995; and Sanders et al., Am J Respir Cell Mol Biol, 2017; 56 (1): 50-61). Full-length caveolin-1 protein may be a less desirable pharmaceutical candidate due to inherent concerns for protein drugs, including stability, delivery, cost, and autoimmunogenicity. Therefore, fragments of caveolin-1, such as the caveolin scaffolding domain peptides (CSPs), have been studied and found to be effective substitutes for the full-length caveolin-1 protein. In particular, the 20-mer form of CSP has been shown to prevent, limit, or reverse fibrosis in animal models, and a seven amino acid fragment of CSP named CSP-7 was sufficiently effective in promoting the reduction of fibrosis both in vitro and in vivo (Marudamuthu et al., Sci Transl Med, 2019; 11 (522), eaat2848). As a result, CSPs have been identified as promising therapeutic agents for the treatment of fibrotic diseases such as IPF.

Biomarkers associated with caveolin-1 peptide therapy in patients with IPF have yet to be identified. These biomarkers could be used to determine or predict efficacy of caveolin-1 peptide therapy. These biomarkers could also be used to determine optimal dosing of caveolin-1 peptide therapy in patients with IPF.

Thus, there is a need in the art to identify biomarkers associated with caveolin-1 peptide therapy in patients with interstitial lung disease such as IPF.

### SUMMARY OF THE DISCLOSURE

The present disclosure provides a method for treating a subject with caveolin-1 peptide therapy, wherein the subject is suffering from fibrosis, the method comprising the steps of: (a) obtaining or having obtained a biological sample from the subject; (b) treating or having treated a cell of the biological sample with a caveolin-1 peptide or derivative thereof; (c) measuring an expression level of a biomarker in the cell; and (d) comparing the expression level of the biomarker to a control sample; wherein the biomarker is myeloid-derived growth factor (MYDGF), soluble RAGE, phosphorylated mothers against decapentaplegic homolog 2/3 (pSMAD2/3), platelet-derived growth factor receptor beta (PDGFRβ), galectin-7 (LGALS7), interleukin-11 (IL-11), matrix metalloproteinase-2 (MMP-2), chemokine ligand 7 (CXCL-7), soluble CD163, phosphorylated mTOR (p-mTOR), phosphorylated PDGFRβ (pPDGFRβ), prolifin (PROF1), calmodulin 2 (CALM2), calreticulin (CALR), peptidyl-prolyl cis-trans isomerase A (PPIA), or eukaryotic translation initiation factor 5A (EIF5A1); and
if the expression level of MYDGF, PROF1, CALM2, CALR, PPIA, IF5A1, LGALS7, soluble RAGE, or EIF5A1 is increased, then administering caveolin-1 peptide or derivative thereof to the subject; or
if the expression level of PDGFRβ, pPDGFRβ, p-mTOR, CALR, IL-11, MMP-2, CXCL7, soluble CD163, or pSMAD2/3 is decreased, then administering caveolin-1 peptide or derivative thereof to the subject.

The present disclosure also provides a method of identifying an altered expression level of a biomarker associated with caveolin-1 peptide therapy, the method comprising: (a) treating a cell of a subject with interstitial lung disease with a caveolin-1 peptide or derivative thereof; (b) measuring an expression level of a biomarker in the cell; and (c) comparing the expression level of the biomarker to a control sample; wherein the caveolin-1 peptide or derivative thereof modulates the expression level of the biomarker in the cell; and wherein the biomarker is myeloid-derived growth factor (MYDGF), soluble RAGE, phosphorylated mothers against decapentaplegic homolog 2/3 (pSMAD2/3), platelet-derived growth factor receptor beta (PDGFRβ), phosphorylated PDGFRβ (pPDGFRβ), phosphorylated mTOR (p-mTOR), galectin-7 (LGALS7), interleukin-11 (IL-11), matrix metalloproteinase-2 (MMP-2), chemokine ligand 7 (CXCL-7), soluble CD163 (sCD163), prolifin (PROF1), calmodulin 2 (CALM2), calreticulin (CALR), peptidyl-prolyl cis-trans isomerase A (PPIA), or eukaryotic translation initiation factor 5A (EIF5A1). In some embodiments, steps (a)-(c) of the method are repeated one or more times.

In some embodiments, the control sample is a cell obtained from the subject with interstitial lung disease prior to treatment with the caveolin-1 peptide or derivative thereof. In some embodiments, the control sample is a cell obtained from a subject or a population of subjects with interstitial lung disease. In some embodiments, the control sample is a cell obtained from a healthy subject.

In some embodiments, the cell is a fibroblast, a type I alveolar epithelial cell, a type II alveolar epithelial cell, a basal-like cell, a Clara cell, a ciliated cell, a club cell, a goblet cell, a neuroendocrine cell, an endothelial cell, a bialveolar stem cell, a macrophage, an alveolar macrophage, an ionocyte, a pericyte, a mesothelial cell, a mesenchymal cell, a neuroendocrine cell, a myofibroblast, a B-cell, a plasma cell, an innate lymphoid cell, a T-cell, a monocyte, an NK cell, a dendritic cell, or a peripheral blood mononuclear cell (PBMC). In some embodiments, the cell is a fibroblast. In some embodiments, the cell is a basal-like cell. In some embodiments, the cell is treated with the caveolin-1 peptide or derivative thereof ex vivo or in vitro. In some embodiments, the cell is obtained from the subject with interstitial lung disease.

In some embodiments, the caveolin-1 peptide or derivative thereof is FTTFTVT (SEQ ID NO: 3). In some embodiments, the caveolin-1 peptide or derivative thereof is Ac-aaEGKASFTTFTVTKGSaa-NH2 (SEQ ID NO: 4).

In some embodiments, the subject is a human.

In some embodiments, the ILD is idiopathic pulmonary fibrosis, lymphangioleiomyomatosis, nonspecific interstitial pneumonia, idiopathic interstitial pneumonia, cryptogenic organizing pneumonia, acute interstitial pneumonia, respiratory bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced ILD, or occupational ILD. In some embodiments, the ILD is idiopathic pulmonary fibrosis.

In some embodiments, the expression level of the biomarker is measured by 2-D gel electrophoresis, Western blot, mass spectrometry, flow cytometry, quantitative RT-PCR, ELISA, and/or a lateral flow immunoassay.

In some embodiments, the increased expression level of MYDGF, PROF1, CALM2, CALR, PPIA, IF5A1, LGALS7, soluble RAGE, or EIF5A1 is associated with caveolin-1 therapy. In some embodiments, the decreased expression level of PDGFRβ, pPDGFRβ, p-mTOR, CALR, IL-11, MMP-2, CXCL7, sCD163, or pSMAD2/3 is associated with caveolin-1 therapy. In some embodiments, the expression level of MYDGF is increased compared to the control sample. In some embodiments, the expression level of MYDGF is increased by about 1.5-fold, about 2-fold, about 3-fold, about 4-fold, about 5-fold, or more compared to the control sample. In some embodiments, the expression level of soluble RAGE is increased compared to the control sample. In some embodiments, the expression level of soluble RAGE is increased by about 1.5-fold, about 2-fold, about 3-fold, about 4-fold, about 5-fold, or more compared to the control sample. In some embodiments, the expression level of pSMAD2/3 is decreased compared to the control sample. In some embodiments, the expression level of pSMAD2/3 is decreased by about 10%, about 25%, about 50%, about 75%, about 90%, or more compared to the control sample. In some embodiments, the expression level of PDGFRβ is decreased compared to the control sample. In some embodiments, the expression level of PDGFRβ is decreased by about 10%, about 25%, about 50%, about 75%, about 90%, or more compared to the control sample.

In some embodiments, the method further comprises measuring an expression level of an internal control and wherein the expression level of the internal control is unaffected by the caveolin-1 peptide or derivative thereof.

In some embodiments, the present disclosure provides a method of predicting or determining the efficacy of a therapeutically active agent, the method comprising: (a) treating a cell of a subject with interstitial lung disease with the therapeutically active agent; (b) measuring an expression level of a biomarker in the cell of the subject; wherein the biomarker is MYDGF, soluble RAGE, pSMAD2/3, PDGFRβ, pPDGFRβ, p-mTOR, LGALS7, IL-11, MMP-2, CXCL7, sCD163, PROF1, CALM2, CALR, PPIA, or EIF5A1; and (c) comparing the expression level of the biomarker to a control sample. In some embodiments, steps (a)-(c) of the method are repeated one or more times.

In some embodiments, the control sample is a cell obtained from the subject with interstitial lung disease prior to treatment with the therapeutically active agent. In some embodiments, the control sample is a cell obtained from a subject or a population of subjects with interstitial lung disease. In some embodiments, the control sample is a cell obtained from a healthy subject.

In some embodiments, the cell is a fibroblast, a type I alveolar epithelial cell, a type II alveolar epithelial cell, a basal-like cell, a Clara cell, a ciliated cell, a club cell, a goblet cell, a neuroendocrine cell, an endothelial cell, a bialveolar stem cell, a macrophage, an alveolar macrophage, an ionocyte, a pericyte, a mesothelial cell, a mesenchymal cell, a neuroendocrine cell, a myofibroblast, a B-cell, a plasma cell, an innate lymphoid cell, a T-cell, a monocyte, an NK cell, a dendritic cell, or a peripheral blood mononuclear cell (PBMC). In some embodiments, the cell is a fibroblast. In some embodiments, the cell is a basal-like cell. In some embodiments, the cell is treated with the therapeutically active agent ex vivo or in vitro. In some embodiments, the cell is obtained from the subject with interstitial lung disease.

In some embodiments, the therapeutically active agent is a small molecule. In some embodiments, the therapeutically active agent is a biologic. In some embodiments, the biologic is a caveolin-1 peptide or derivative thereof. In some embodiments, the caveolin-1 peptide or derivative thereof is FTTFTVT (SEQ ID NO: 3). In some embodiments, the caveolin-1 peptide or derivative thereof is Ac-aaEGKASFTTFTVTKGSaa-NH2 (SEQ ID NO: 4).

In some embodiments, the interstitial lung disease is idiopathic pulmonary fibrosis, lymphangioleiomyomatosis, nonspecific interstitial pneumonia, idiopathic interstitial bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease. In some embodiments, the interstitial lung disease is idiopathic pulmonary fibrosis.

In some embodiments, the expression level of the biomarker is measured by 2-D gel electrophoresis, Western blot, mass spectrometry, flow cytometry, quantitative RT-PCR, ELISA, and/or a lateral flow immunoassay.

In some embodiments, the increased expression level of MYDGF, PROF1, CALM2, CALR, PPIA, IF5A1, LGALS7, soluble RAGE, or EIF5A1 indicates a favorable response to the therapeutically active agent. In some embodiments, the decreased expression level of PDGFRβ, pPDGFRβ, p-mTOR, pSMAD2/3, IL-11, MMP-2, CXCL7, sCD163, or CALR indicates a favorable response to the therapeutically active agent. In some embodiments, the expression level of MYDGF is increased compared to the control sample. In some embodiments, the expression level of MYDGF is increased by about 1.5-fold, about 2-fold, about 3-fold, about 4-fold, about 5-fold, or more compared to the control sample. In some embodiments, the expression level of soluble RAGE is increased compared to the control sample. In some embodiments, the expression level of soluble RAGE is increased by about 1.5-fold, about 2-fold, about 3-fold, about 4-fold, about 5-fold, or more compared to the control sample. In some embodiments, the expression level of pSMAD2/3 is decreased compared to the control sample. In some embodiments, the expression level of pSMAD2/3 is decreased by about 10%, about 25%, about 50%, about 75%, about 90%, or more compared to the control sample. In some embodiments, the expression level of PDGFRβ is decreased compared to the control sample. In some embodiments, the expression level of PDGFRβ is decreased by about 10%, about 25%, about 50%, about 75%, about 90%, or more compared to the control sample.

In some embodiments, the method further comprises measuring an expression level of an internal control and wherein the expression level of the internal control is unaffected by the therapeutically active agent.

In some embodiments, the present disclosure provides a method of predicting or determining the efficacy of a caveolin-1 peptide or derivative thereof, the method comprising: (a) treating a biological sample from a subject with fibrosis with the caveolin-1 peptide or derivative thereof; (b) measuring an expression level of a biomarker in the biological sample from the subject; wherein the biomarker is MYDGF, soluble RAGE, pSMAD2/3, PDGFRβ, pPDGFRβ, p-mTOR, LGALS7, IL-11, MMP-2, CXCL7, sCD163, PROF1, CALM2, CALR, PPIA, or IF5A1; and (c) comparing the expression level of the biomarker to a control sample. In some embodiments, steps (a)-(c) of the method are repeated one or more times.

In some embodiments, the control sample is a biological sample obtained from the subject with fibrosis prior to treatment with the caveolin-1 peptide or derivative thereof. In some embodiments, the control sample is a biological sample obtained from a subject or a population of subjects with fibrosis. In some embodiments, the control sample is a biological sample obtained from a healthy subject.

In some embodiments, the biological sample is bronchoalveolar lavage fluid (BALF), lung tissue, fibroblasts, type I alveolar epithelial cells, type II alveolar epithelial cells, basal cells, Clara cells, ciliated cells, club cells, goblet cells, neuroendocrine cells, endothelial cells, bialveolar stem cells, macrophages, alveolar macrophages, ionocytes, pericytes, mesothelial cells, mesenchymal cells, neuroendocrine cells, myofibroblasts, B-cells, plasma cells, innate lymphoid cells, T-cells, monocytes, NK cells, dendritic cells, or PBMCs. In some embodiments, the biological sample is fibroblasts. In some embodiments, the biological sample is basal-like cells. In some embodiments, the biological sample is lung tissue. In some embodiments, the biological sample is treated with the caveolin-1 peptide or derivative thereof ex vivo or in vitro. In some embodiments, the biological sample is obtained from the subject with fibrosis.

In some embodiments, the caveolin-1 peptide or derivative thereof is FTTFTVT (SEQ ID NO: 3). In some embodiments, the caveolin-1 peptide or derivative thereof is Ac-aaEGKASFTTFTVTKGSaa-NH2 (SEQ ID NO: 4).

In some embodiments, the fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

In some embodiments, the interstitial lung disease is idiopathic pulmonary fibrosis, lymphangioleiomyomatosis, nonspecific interstitial pneumonia, idiopathic interstitial bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease. In some embodiments, the interstitial lung disease is idiopathic pulmonary fibrosis.

In some embodiments, the expression level of the biomarker is measured by 2-D gel electrophoresis, Western blot, mass spectrometry, flow cytometry, quantitative RT-PCR, ELISA, and/or a lateral flow immunoassay.

In some embodiments, the increased expression level of MYDGF, PROF1, CALM2, CALR, PPIA, IF5A1, LGALS7, and soluble RAGE indicates a favorable response to the caveolin-1 peptide or derivative thereof. In some embodiments, the decreased expression level of PDGFRβ, pPDGFRβ, p-mTOR, CALR, IL-11, MMP-2, CXCL7, sCD163, or pSMAD2/3 indicates a favorable response to the caveolin-1 peptide or derivative thereof. In some embodiments, the expression level of MYDGF is increased compared to the control sample. In some embodiments, the expression level of MYDGF is increased by about 1.5-fold, about 2-fold, about 3-fold, about 4-fold, about 5-fold, or more compared to the control sample. In some embodiments, the expression level of soluble RAGE is increased compared to the control sample. In some embodiments, the expression level of soluble RAGE is increased by about 1.5-fold, about 2-fold, about 3-fold, about 4-fold, about 5-fold, or more compared to the control sample. In some embodiments, the expression level of pSMAD2/3 is decreased compared to the control sample. In some embodiments, the expression level of pSMAD2/3 is decreased by about 10%, about 25%, about 50%, about 75%, about 90%, or more compared to the control sample. In some embodiments, the expression level of PDGFRβ is decreased compared to the control sample. In some embodiments, the expression level of PDGFRβ is decreased by about 10%, about 25%, about 50%, about 75%, about 90%, or more compared to the control sample.

In some embodiments, the method further comprises measuring an expression level of an internal control and wherein the expression level of the internal control is unaffected by the caveolin-1 peptide or derivative thereof.

In some embodiments, the present disclosure provides a method of predicting or determining the efficacy of a caveolin-1 peptide or derivative thereof in a subject with fibrosis, the method comprising: (a) obtaining a first biological sample from the subject prior to treatment with the caveolin-1 peptide or derivative thereof; (b) administering the caveolin-1 peptide or derivative thereof to the subject; (c) obtaining a second biological sample from the subject following treatment with the caveolin-1 peptide or derivative thereof; (d) measuring an expression level of a biomarker in the first biological sample and the second biological sample; wherein the biomarker is MYDGF, soluble RAGE, pSMAD2/3, PDGFRβ, pPDGFRβ, p-mTOR, LGALS7, IL-11, MMP-2, CXCL7, sCD163, PROF1, CALM2, CALR, PPIA, or IF5A1; and (e) comparing the expression level of the biomarker in the first biological sample to the expression level of the biomarker in the second biological sample. In some embodiments, step (a) of the method further comprises determining the expression level of the biomarker in the first biological sample compared to a control sample prior to treatment with the caveolin-1 peptide or derivative thereof. In some embodiments, the expression level of the biomarker in the first biological sample of step (a) is used to determine an optimal dose of the caveolin-1 peptide or derivative thereof administered in step (b).

In some embodiments, the control sample is a biological sample obtained from a subject or a population of subjects with fibrosis.

In some embodiments, the biological sample is serum, plasma, BALF, lung tissue, fibroblasts, type I alveolar epithelial cells, type II alveolar epithelial cells, basal cells, Clara cells, ciliated cells, club cells, goblet cells, neuroendocrine cells, endothelial cells, bialveolar stem cells, macrophages, alveolar macrophages, ionocytes, pericytes, mesothelial cells, mesenchymal cells, neuroendocrine cells, myofibroblasts, B-cells, plasma cells, innate lymphoid cells, T-cells, monocytes, NK cells, dendritic cells, or PBMCs. In some embodiments, the biological sample is serum. In some embodiments, the biological sample is plasma. In some embodiments, the biological sample is fibroblasts, basal-like cells, or PBMCs.

In some embodiments, the caveolin-1 peptide or derivative thereof is FTTFTVT (SEQ ID NO: 3). In some embodiments, the caveolin-1 peptide or derivative thereof is Ac-aaEGKASFTTFTVTKGSaa-NH2 (SEQ ID NO: 4).

In some embodiments, the fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

In some embodiments, the interstitial lung disease is idiopathic pulmonary fibrosis, familial pulmonary fibrosis, idiopathic nonspecific interstitial pneumonia, conventional interstitial pneumonia, cryptogenic organizing pneumonia, or sarcoidosis. In some embodiments, the interstitial lung disease is idiopathic pulmonary fibrosis.

In some embodiments, the expression level of the biomarker in the first and second biological sample is measured by 2-D gel electrophoresis, Western blot, mass spectrometry, flow cytometry, quantitative RT-PCR, ELISA, and/or a lateral flow immunoassay.

In some embodiments, the increased expression level of MYDGF, PROF1, CALM2, CALR, PPIA, IF5A1, LGALS7, soluble RAGE, or EIF5A1 indicates a favorable response to the caveolin-1 peptide or derivative thereof. In some embodiments, the decreased expression level of PDGFRβ, pPDGFRβ, p-mTOR, CALR, IL-11, MMP-2, CXCL7, sCD163, or pSMAD2/3 indicates a favorable response to the caveolin-1 peptide or derivative thereof.

In some embodiments, the caveolin-1 peptide or derivative thereof is administered to the subject at a dose of about 0.01 mg/kg to about 250 mg/kg. In some embodiments, the caveolin-1 peptide or derivative thereof is administered to the subject at a dose of about 0.05 mg/kg to about 50 mg/kg.

In some embodiments, the caveolin-1 peptide or derivative thereof is administered to the subject through inhalation, intravenously, subcutaneously, orally, intraperitoneally, sublingually, buccally, or intramuscularly.

In some embodiments, the method comprises administering the caveolin-1 peptide or derivative thereof to the subject once per day, once per week, twice per week, three times per week, five times per week, once every two weeks, or once per month.

In some embodiments, the second biological sample is obtained from the subject one hour, three hours, six hours, twelve hours, one day, two days, three days, four days, five days, one week, two weeks, three weeks, one month, six months, or one year following administration of the caveolin-1 peptide or derivative thereof.

In some embodiments, the method further comprises obtaining one or more additional biological samples following administration of the caveolin-1 peptide or derivative thereof. In some embodiments, the expression level of the biomarker in the one or more additional biological samples is increased compared to the expression level of the biomarker in the first biological sample; and wherein the increased expression level of the biomarker in the one or more additional biological samples indicates a favorable response to the caveolin-1 peptide or derivative thereof. In some embodiments, the expression level of the biomarker in the one or more additional biological samples is decreased compared to the expression level of the biomarker in the first biological sample; and wherein the decreased expression level of the biomarker in the one or more additional biological samples indicates a favorable response to the caveolin-1 peptide or derivative thereof.

In some embodiments, the expression level of MYDGF in the second biological sample is increased compared to the first biological sample. In some embodiments, the expression level of MYDGF in the second biological sample is increased by about 1.5-fold, about 2-fold, about 3-fold, about 4-fold, about 5-fold, or more compared to the first biological sample. In some embodiments, the expression level of soluble RAGE in the second biological sample is increased compared to the first biological sample. In some embodiments, the expression level of soluble RAGE in the second biological sample is increased by about 1.5-fold, about 2-fold, about 3-fold, about 4-fold, about 5-fold, or more compared to the first biological sample. In some embodiments, the expression level of pSMAD2/3 is decreased compared to the control sample. In some embodiments, the expression level of pSMAD2/3 is decreased by about 10%, about 25%, about 50%, about 75%, about 90%, or more compared to the control sample. In some embodiments, the expression level of PDGFRβ in the second biological sample is decreased in the second biological sample compared to the first biological sample. In some embodiments, the expression level of PDGFRβ in the second biological sample is decreased by about 10%, about 25%, about 50%, about 75%, about 90%, or more compared to the first biological sample.

In some embodiments, the expression level of the biomarker in the second biological sample is used to determine an optimal dose of the caveolin-1 peptide or derivative thereof.

### BRIEF DESCRIPTION OF THE DRAWINGS

FIG. 1A and FIG. 1B show MYDGF expression in epithelial cells from IPF patients. Epithelial cells were treated with 1% DMSO vehicle, CSP-7 (10 μM), Var55 (10 μM), control peptide 1 (CP1, 10 μM), control peptide 2 (CP2, 10 μM), pirfenidone (500 μM), or nintedanib (100 nM). MYDGF expression was measured in epithelial cell supernatant by ELISA.

FIG. 2 shows a schematic of testing therapeutic agents on human IPF lung tissue ex vivo. Fresh lungs are obtained from human subjects with IPF and lung cores are drilled and transferred to a Krumdieck slicer. Precision cut lung slices (PCLSs) are kept alive with human serum and culture media for up to one month and therapeutic agents, e.g., CSP-7, are directly tested on the diseased organ made up of multiple cell types.

FIG. 3A and FIG. 3B show soluble RAGE expression in the supernatant of IPF precision cut lung slices (PCLSs). PCLSs were either untreated (UT) or treated with CSP-7 (10 μM or 100 μM), control peptide (CP, 100 μM), TGFβ (2 ng/ml), or pirfenidone (Pirf, 500 μM) and nintedanib (Nin, 100 nM). FIG. 3C shows the effect of different concentrations of CSP-7 (0.001 μM to 100 μM) on soluble RAGE expression in IPF PCLSs. Soluble RAGE expression was measured in the supernatant of IPF PCLSs on day 2 and 4 following treatment by ELISA (FIGS. 3A-3C).

FIG. 4A shows lactate dehydrogenase (LDH) activity in end-stage IPF precision cut lung slices (PCLSs). PCLSs were either untreated or treated with CSP-7 (10 μM or 100 μM), control peptide (CP, 100 μM), TGFβ (2 ng/mL), or pirfenidone (Pirf, 500 μM) and nintedanib (Nin, 100 nM). LDH activity was measured in the supernatant of IPF PCLSs on day 2 (left panel) or day 4 (right panel) following treatment. FIG. 4B shows LDH activity in end-stage IPF PCLSs either untreated (UT) or treated with different concentrations of CSP-7 (0.001 μM to 100 μM). LDH activity was measured in the supernatant of IPF PCLSs on day 2 (left panel) or day 4 (right panel) following treatment.

FIG. 5 shows soluble RAGE expression in plasma of healthy subjects on day 1 and day 13 following inhalation of CSP-7. Soluble RAGE expression was measured by ELISA.

FIG. 6A shows PDGFRβ expression in end-stage IPF precision cut lung slices (PCLSs) from three biological replicates. PCLSs were treated with CSP-7 (10 UM or 100 μM), control peptide (CP, 100 μM), TGFβ (2 ng/ml), pirfenidone (500 μM), or nintedanib (100 nM). Protein expression was determined by Western blot. Beta actin was used as an endogenous control. FIG. 6B shows relative PDGFRβ protein expression in end-stage IPF precision cut lung slices (PCLSs). PCLSs were treated with CSP-7 (10 UM or 100 μM), control peptide (CP, 100 μM), TGFβ (2 ng/mL), or pirfenidone (Pirf, 500 μM) and nintedanib (Nint, 100 nM). Relative protein expression was determined using the data shown in FIG. 6A. *P≤0.05 and **P≤0.01.

FIG. 7A shows pSMAD2/3 phosphorylation in IPF fibroblasts. IPF fibroblasts were treated with DMEM, 1% DMSO vehicle, CSP-7 (10 μM), Var55 (10 μM), pirfenidone (500 μM), or nintedanib (100 nM). SMAD2/3 phosphorylation was determined by Western blot 24 hours following treatment. FIG. 7B shows pSMAD2/3 phosphorylation in IPF epithelial cells. IPF epithelial cells were treated with DMEM (control), Var55 (100 μM), nintedanib (100 nM), control peptide 1 (CP1, 100 μM), or control peptide 2 (CP2, 100 μM). SMAD2/3 phosphorylation was determined by Western blot 24 hours following treatment. Phosphorylated SMAD2/3 protein levels were determined by normalizing to total SMAD2/3 protein (FIG. 7A and FIG. 7B).

FIG. 8 shows galectin-7 in basal-like cells and fibroblasts derived from normal healthy subjects or subjects with idiopathic pulmonary fibrosis (IPF). The cells were treated with DMSO vehicle, CSP-7 (10 μM), Var55 (10 μM), control peptide 1 (CP1, 10 μM), control peptide 2 (CP2, 10 μM), pirfenidone (500 μM), or nintedanib (100 nM).

FIG. 9 shows collagen in total lung homogenates of saline (SAL)- or bleomycin (BLM)-treated aged mice administered control peptide (CP) or CSP-7 through dry powder inhalation (DPI). ****P≤0.0001.

FIG. 10A shows total SMAD (tSMAD, top panel) and phosphorylated SMAD (pSMAD, bottom panel) in total lung homogenates of bleomycin (BLM)-treated aged mice either untreated or administered control peptide (CP) or CSP-7 through dry powder inhalation or CSP-7 through intraperitoneal injection (IP).

FIG. 10B shows galectin-7 in total lung homogenates of bleomycin (BLM)-treated aged mice either untreated or administered control peptide (CP) or CSP-7 through dry powder inhalation or CSP-7 through intraperitoneal injection (IP).

FIG. 11A shows phosphorylation of EGFR (p-EGFR), SRC (p-SRC), MEK1/2 (p-MEK 1/2) and Raptor (p-Raptor) in IPFs (top panel) or normal lung fibroblasts (bottom panel) either untreated or treated with DMSO, control peptide (CP, 100 μM), or CSP-7 (10 μM).

FIG. 11B shows phosphorylation of PDGFRβ (p-PDGFRβ) and mTOR (p-mTOR) in IPFs either untreated or treated with DMSO, control peptide (CP, 100 μM), or CSP-7 (10 μM).

FIG. 11C provides a summary of the reverse phase protein array that assessed receptor tyrosine kinase (RTK) signaling, metabolic signaling, invasion-associated markers, and histone deacetylases (HDAC). The p-values were calculated by comparing IPFs treated with control peptide (CP) or CSP-7.

FIG. 12A shows IL-11 expression levels in bronchoalveolar lavage fluid (BALF) from mice treated with saline, bleomycin (BLM), BLM and CSP-7, BLM and control peptide (CP), or BLM and CSP-7 administered by intraperitoneal (IP) injection. Mice in the preventative group received three daily doses of CP or CSP-7 prior to administration of BLM.

FIG. 12B shows IL-11 expression levels in bronchoalveolar lavage fluid (BALF) from mice treated with saline, bleomycin (BLM), BLM and CSP-7, BLM and control peptide (CP), or BLM and CSP-7 administered by intraperitoneal (IP) injection. Mice in the therapeutic group received three daily doses of CP or CSP-7 following administration of BLM.

FIG. 13A shows soluble CD163 (sCD163) expression in the supernatant of human lung macrophages isolated from subjects with idiopathic pulmonary fibrosis (IPF). IPF macrophages were either left untreated or treated with 0.1 μM or 1 μM of CSP-7 for 24 hours prior to analysis for sCD163. Each line represents a biological replicate of lung macrophages from an individual subject with IPF.

FIG. 13B shows MMP-2 expression in the supernatant of human lung macrophages isolated from subjects with idiopathic pulmonary fibrosis (IPF). IPF lung macrophages were either left untreated or treated with 0.1 μM or 1 μM of CSP-7 for 24 hours prior to analysis for MMP-2. Each line represents a biological replicate of lung macrophages from an individual subject with IPF.

FIG. 14 shows CXCL7 expression in the supernatant of human lung macrophages isolated from healthy control subjects or subjects with idiopathic pulmonary fibrosis (IPF). Lung macrophages were either left untreated or treated with Var55. Each line represents a biological replicate of lung macrophages from an individual control subject (triangles) or an individual with IPF (circles).

### DETAILED DESCRIPTION

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized enzyme-linked immunosorbent assay. As appropriate, procedures involving the use of commercially available kits and reagents are generally carried out in accordance with manufacturer defined protocols and/or parameters unless otherwise noted.

As used in the specification and the appended claims, the terms “a,” “an” and “the” include both singular and the plural referents unless the context clearly dictates otherwise.

The term “about” when immediately preceding a numerical value means ± up to 20% of the numerical value. In some embodiments, “about” a numerical value means ± up to 20%, ± up to 19%, ± up to 18%, ± up to 17%, ± up to 16%, ± up to 15%, ± up to 14%, ± up to 13%, ± up to 12%, ± up to 11%, ± up to 10%, ± up to 9%, ± up to 8%, ± up to 7%, ± up to 6%, ± up to 5%, ± up to 4%, ± up to 3%, ± up to 2%, ± up to 1%, ± up to less than 1%, or any other value or range of values therein, of the numerical value.

The term “biomarker” refers to an indicator of treatment with a therapeutically active agent, e.g., a caveolin-1 peptide or derivative thereof, in a subject with a disease or disorder. In some embodiments, the biomarker is used to predict or determine efficacy in a subject with a disease or disorder. In some embodiments, the biomarker can be detected or measured in a biological sample obtained from a subject with a disease or disorder. In some embodiments, the biomarker can be detected or measured in a biological sample treated in vitro with a therapeutically active agent, e.g., a caveolin-1 peptide or derivative thereof. In some embodiments, the biomarker can be detected or measured in a biological sample obtained from a subject treated in vivo with a therapeutically active agent, e.g., a caveolin-1 peptide or derivative thereof. Examples of biomarkers include, but are not limited to, DNA, RNA, protein, phosphorylated proteins, carbohydrate, or glycolipid-based biomarkers.

As used herein, a protein denoted with the letter “p” refers to a phosphorylated protein, e.g., phosphorylated SMAD2/3 as pSMAD2/3. Methods for detecting phosphorylated proteins are well-known in the art.

The term “detection” or “detecting” as used herein refers to quantitative or qualitative detection of a biomarker in a biological sample. The term “detection” or “detecting” includes any means of detecting the expression level of a biomarker in a biological sample, e.g., an immunoassay, qRT-PCR, or mass spectrometry.

The term “identification” or “identifying” as used herein refers to quantitative or qualitative detection of a biomarker in a biological sample. The term “identification” or “identifying” includes any means of identifying the expression level of a biomarker in a biological sample, e.g., an immunoassay, qRT-PCR, or mass spectrometry.

The term “section” of a tissue sample refers to a single part or piece of a tissue sample, e.g., a thin slice of tissue or cells cut from a tissue sample. In some embodiments, the same section of tissue sample is analyzed at morphological and molecular levels, or is analyzed with respect to both protein and nucleic acid for the biomarkers described herein. In some embodiments, multiple sections of tissue are analyzed at morphological and molecular levels, or are analyzed with respect to both protein and nucleic acid for the biomarkers described herein.

The term “polynucleotide” or “nucleic acid” refers to a polymer of nucleotide monomers covalently bonded in a chain. Exemplary nucleic acids include DNA and RNA.

The term “amino acid” refers to structural units (monomers) that make up a protein, polypeptide, or peptide. The term “polypeptide” or “protein” includes any polymer of amino acids or amino acid residues. A “peptide” is a small polypeptide of sizes less than about 15 to 20 amino acid residues. The term “amino acid sequence” refers to a series of amino acids or amino acid residues.

Methods for determining sequence similarity or identity between two or more nucleic acid sequences or two or more amino acid sequences are known in the art. Sequence similarity or identity may be determined using standard techniques, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection. Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). An exemplary BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional algorithm is gapped BLAST as reported by Altschul et al, (1997) Nucleic Acids Res. 25, 3389-3402. Unless indicated otherwise, calculation of percent identity is performed in the instant disclosure using the BLAST algorithm available at the world wide web address: blast.ncbi.nlm.nih.gov/Blast.cgi.

The term “antibody” refers to an immunoglobulin which may be derived from natural sources or synthetically produced, in whole or in part. Examples of antibodies include, but are not limited to, monoclonal antibodies, polyclonal antibodies, antibodies with polyepitopic specificity, single chain antibodies, multi-specific antibodies and fragments of antibodies (e.g., scFv, diabodies, triabodies, minibodies). Such antibodies can be chimeric, humanized, human and synthetic.

As used herein, the terms “treat,” “treating,” or “treatment”, and grammatical variants thereof, have the same meaning as commonly understood by those of ordinary skill in the art. In some embodiments, these terms may refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (e.g. not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject in need of treatment may thus be a subject already afflicted with the disease or disorder in question. The terms “treat,” “treating,” or “treatment” includes inhibition or reduction of an increase in severity of a pathological state or symptoms relative to the absence of treatment, and is not necessarily meant to imply complete cessation of the relevant disease or condition. The terms “treat,” “treating,” or “treatment” can also refer to providing a therapeutically active agent, e.g., a caveolin-1 peptide or derivative thereof, to a biological sample obtained from a subject with a disease or disorder.

As used herein, the term “pharmaceutically acceptable carrier or excipient” includes without limitation any adjuvant, carrier, glidant, sweetening agent, diluent, preservative, dye/colorant, flavor enhancer, surfactant, wetting agent, dispersing agent, suspending agent, stabilizer, isotonic agent, solvent, surfactant, and/or emulsifier. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

The term “interstitial lung disease” or “ILD” refers to a group of lung diseases affecting the interstitium (the tissue and space around the air sacs of the lungs). ILD can be classified according to a suspected or known cause, or can be idiopathic. For example, ILD can be classified as caused by inhaled substances (inorganic or organic), drug-induced (e.g., antibiotics, chemotherapeutic drugs, antiarrhythmic agents, statins), associated with connective tissue disease (e.g., systemic sclerosis, polymyositis, dermatomyositis, systemic lupus erythematous, rheumatoid arthritis), associated with pulmonary infection (e.g., atypical pneumonia, pneumocystis pneumonia, tuberculosis, Chlamydia trachomatis, Respiratory Syncytial Virus, COVID-19), associated with a malignancy (e.g., lymphangitic carcinomatosis), or can be idiopathic (e.g., sarcoidosis, idiopathic pulmonary fibrosis, Hamman-Rich syndrome, or antisynthetase syndrome).

The term “idiopathic pulmonary fibrosis” or “IPF” refers to a chronic, progressive form of lung disease characterized by fibrosis of the supporting framework (interstitium) of the lungs. Microscopically, lung tissue from patients having IPF shows a characteristic set of histologic/pathologic features known as usual interstitial pneumonia, characterized by a heterogeneous, variegated appearance with alternating areas of healthy lung, interstitial inflammation, fibrosis, and honeycomb change. By definition, the term IPF is used when the cause of the pulmonary fibrosis is unknown (“idiopathic”). Symptoms typically include gradual onset of shortness of breath and a dry cough. Other changes may include feeling tired, and abnormally large and dome shaped finger and toenails (nail clubbing). Complications may include pulmonary hypertension, heart failure, pneumonia, or pulmonary embolism.

The term “optimal dose” refers to an amount of therapeutically active agent effective to “alleviate” or “treat” a disease or disorder in a subject. An optimal dose of a therapeutically active agent may vary according to factors such as the disease state, age, sex, and weight of the individual. An optimal dose is also one in which any toxic or detrimental effects of the therapeutically active agent are outweighed by the therapeutically beneficial effects.

The phrase “elevated expression level” or “increased expression level” refers to an increased expression of a biomarker (e.g., mRNA or protein biomarker) in a biological sample from a subject treated with a therapeutically active agent compared to a control sample.

The phrase “reduced expression level” or “decreased expression level” refers to a decreased expression of a biomarker (e.g., mRNA or protein biomarker) in a biological sample from a subject treated with a therapeutically active agent compared to a control sample.

### Therapeutically Active Agents

The present disclosure provides methods of identifying an altered expression level of a biomarker associated with caveolin-1 peptide therapy. In some embodiments, the biomarkers described herein are used to determine or predict efficacy of a therapeutically active agent in a subject with a disease or disorder. In some embodiments, the biomarkers described herein are used to determine or predict optimal dosing of a therapeutically active agent in a subject with a disease or disorder. In some embodiments, the disease or disorder is interstitial lung disease, e.g., idiopathic pulmonary fibrosis. In some embodiments, the therapeutically active agent is a caveolin-1 peptide or a derivative thereof.

The term “therapeutically active agent” refers to any type of drug, medicine, or pharmaceutical, which is used to treat, control, or prevent any disease, disorder, or undesirable medical condition in a mammalian subject. In some embodiments, the therapeutically active agent is a small molecule. In some embodiments, the therapeutically active agent is a biologic. In some embodiments, the therapeutically active agent is a caveolin-1 peptide or derivative thereof. In some embodiments, the therapeutically active agent is CSP-7. In some embodiments, the therapeutically active agent is Var55.

### Caveolin-1 Peptides and Derivatives Thereof

In some embodiments, the therapeutically active agent is a caveolin-1 peptide or derivative thereof. Native human caveolin-1 has 178 amino acids (see, SEQ ID NO: 1 in Table 1 below) and a molecular weight of 22 kDa. Caveolin-1 is an integral membrane protein associated with endocytosis, extracellular matrix organization, cholesterol distribution, cell migration, and signaling. See, Boscher and Nabi, Adv Exp Med Biol, 2012; 729-29-50. Caveolin-1 regulates several important signaling components, including TGF-β and ERK1/2, and has a homeostatic function in the fibrosis process by participating in a series of key regulatory pathways (see, Shihata et al., Front. Pharmacol. 2017; 8:567; and Gvaramia et al., Matrix Biol, 2013: 32 (6): 307-315). While non-lipid raft associated internalization of TGF-βR increases TGF-β signaling, caveolin-1-associated internalization increases TGF-βR degradation, thus decreasing or abolishing TGF-β signaling (Zhang et al, JBC, 2005; 280:12239-45; Di Guglielmo et al., Nat Cell Biol, 2003; 5:410-21). Endogenous caveolin-1 was found to be constitutively suppressed in fibrotic lungs of a bleomycin-induced animal model of IPF, as well as IPF patients (see, Wang et al., J Exp Med, 2006; 203 (13): 2895-2906; Sanders et al., PLOS One, 2015; 10 (2), e0116995; and Sanders et al., Am J Respir Cell Mol Biol, 2017; 56 (1): 50-61). Derivatives of the caveolin-1 peptide include caveolin-1 scaffolding domain peptides (CSP).

In some embodiments, the therapeutically active agent is a caveolin-1 scaffolding domain peptide (CSP) or derivative thereof. The caveolin-1 scaffolding domain (CSD) is comprised of the amino acids 82-101 of caveolin-1 (see, SEQ ID NO: 2 in Table 1 below). The CSD of caveolin-1 plays a critical role in caveolin-1 dimerization as well as regulation of diverse signaling intermediates, many of which are implicated in the pathogenesis of lung and tissue fibrosis (Shetty et al., Am J Respir Cell Mol Biol 2012; 47:474-83; Fridolfsson et al., FASEB J 2014; 28:3823-31; Degryse et al., Am J Physiol Cell Mol Physiol 2010; 299: L442-L452; and Egger et al., PLos One, 2013; 8: e63432). The CSD domain of caveolin-1 has demonstrated inhibition of Wnt-signaling, β-catenin-mediated transcription, activation of SRC, EGFR, MEK1 and ERK-2 and various other factors implicated in pro-fibrotic signaling (see, Shetty et al., Am J Respir Cell Mol Biol 2012; 47:474-83; Bhandary et al., Am J Physiol Cell Mol Physiol 2012; 302: L463-L473; Bhandary et al., Am J Pathol 2013; 183:131-143; Fridolfsson et al., FASEB J 2014; 28:3823-31; Degryse et al., Am J Physiol Cell Mol Physiol 2010; 299: L442-L452; and Fiddler et al., Ann Am Thorac Soc, 2016; 13:1430-2). Endogenous CSD domains can form homodimers with other caveolin-1 proteins and interact with proteins that have a caveolin binding domain sequence (CBD) motif. It is estimated that up to 30% of all endogenous proteins have CBD motifs and the caveolin-1 CSD domain is hypothesized to provide stability to these proteins (see, Marudamuthu et al., Am J Pathol 2015; 185:55-68). Previous studies have shown that CSPs prevent, limit, or reverse pulmonary fibrosis in animal models (see, Marudamuthu et al., Sci Transl Med, 2019; 11 (522), eaat2848). Treatment with the CSP 20-mer (the full-length CSD of caveolin-1) resulted in reduced lung αSMA and lung epithelial apoptosis, reduced collagen deposition, and down-regulation of expression of profibrogenic signaling molecules (see, Bhandary et al., Am J Phys Lung Cell Mol Phys, 2012, 302 (5), L463-L473; Razani et al., JBC, 2001, 276 (9), 6727-6738; and Lee et al., Biochem Biophys Res Commun, 2007, 359 (2): 385-390).

In some embodiments, the therapeutically active agent is CSP-7. CSP-7 is a seven amino acid fragment of the human CSD of caveolin-1. CSP-7 has been shown to be effective in reducing fibrosis in both in vitro and in vivo models (see, Marudamuthu et al., Sci Transl Med, 2019; 11 (522), eaat2848). CSP-7 increases p53 protein levels, reduces urokinase plasminogen activator (uPA) and uPA receptor (uPAR), and increases plasminogen activator inhibitor-1 (PAI-1) expression in cells, such as fibrotic lung fibroblasts. See, WO 2014/145389A1; and WO 2020/055812 A1, which are hereby incorporated by reference in their entireties.

In some embodiments, the therapeutically active agent is Var55.

Exemplary amino acid sequences of the caveolin-1 peptide or derivatives thereof are shown below in Table 1. Upper case letters denote L-amino acids and lower case letters denote D-amino acids. The term “Ac” refers to an acetyl group and the term “NH2” refers to an amino group. The “O” denotes ornithine.

TABLE 1
Caveolin-1 Peptides and Derivatives Thereof

| | | SEQ ID |
|---|---|---|
| Name | Amino Acid Sequence | NO. |
| Caveolin-1 | MSGGKYVDSEGHLYTVPIREQGNIYKPNNKA | 1 |
| | MADELSEKQVYDAHTKEIDLVNRDPKHLND | |
| | DVVKIDFEDVIAEPEGTHSFDGIWKASFTTFT | |
| | VTKYWFYRLLSALFGIPMALIWGIYFAILSFL | |
| | HIWAVVPCIKSFLIEIQCISRVYSIYVHTVCDPL | |
| | FEAVGKIFSNVRINLQKEI | |
| Caveolin-1 | DGIWKASFTTFTVTKYWFYR | 2 |
| scaffolding domain | | |
| (CSD) | | |
| CSP-7 | FTTFTVT | 3 |
| Var55 | Ac-aaEGKASFTTFTVTKGSaa-NH2 | 4 |
| Caveolin-1 derivative | KASFTTFTVTKGS | 5 |
| Caveolin-1 derivative | aaEGKASFTTFTVTKGSaa | 6 |
| Caveolin-1 derivative | OASFTTFTVTOS | 7 |
| Caveolin-1 derivative | aaEGKASFTTFTVTKGSaa-NH2 | 8 |
| Caveolin-1 derivative | ASFTTFTVT | 9 |
| Caveolin-1 derivative | OASFTTFTVTOS-NH2 | 10 |
| Caveolin-1 derivative | FTTFTVT-NH2 | 11 |
| Caveolin-1 derivative | FTTFTVTK-NH2 | 12 |
| Caveolin-1 derivative | KASFTTFTVTK-NH2 | 13 |
| Caveolin-1 derivative | Ac-KASFTTFTVTK-NH2 | 14 |
| Caveolin-1 derivative | OASFTTFTVTK-NH2 | 15 |
| Caveolin-1 derivative | Ac-OASFTTFTVTK-NH2 | 16 |
| Caveolin-1 derivative | Ac-KASFTTFTVTKGS-NH2 | 17 |
| Caveolin-1 derivative | DSGKASFTTFTVTK-NH2 | 18 |
| Caveolin-1 derivative | Ac-DSGKASFTTFTVTK-NH2 | 19 |
| Caveolin-1 derivative | Ac-OASFTTFTVTOS-NH2 | 20 |

TABLE 2
Additional Illustrative Caveolin-1 Peptides and
Derivatives Thereof

| Amino acid sequence | SEQ ID NO |
|---|---|
| KASFTTFTVTKGS-NH2 | 21 |
| KASFTTFTVTKY | 22 |
| IWKASFTTFTVT | 23 |
| SFTTFTVTKYWFY | 24 |
| KASFTTFTVTKYW | 25 |
| IWKASFTTFTVTK | 26 |
| FTTFTVTKYWFYRL | 27 |
| ASFTTFTVTKYWFY | 28 |
| WKASFTTFTVTKYW | 29 |
| GIWKASFTTFTVTK | 30 |
| FTTFTVTKYWFYRLL | 31 |
| ASFTTFTVTKYWFYR | 32 |
| WKASFTTFTVTKYWF | 33 |
| GIWKASFTTFTVTKY | 34 |
| FDGIWKASFTTFTVT | 35 |
| SFTTFTVTKYWFYRLL | 36 |
| KASFTTFTVTKYWFYR | 37 |
| IWKASFTTFTVTKYWF | 38 |
| DGIWKASFTTFTVTKY | 39 |
| SFDGIWKASFTTFTVT | 40 |
| SFTTFTVTKYWFYRLLS | 41 |
| KASFTTFTVTKYWFYRL | 42 |
| IWKASFTTFTVTKYWFY | 43 |
| DGIWKASFTTFTVTKYW | 44 |
| SFDGIWKASFTTFTVTK | 45 |
| FTTFTVTKYWFYRLLSAL | 46 |
| ASFTTFTVTKYWFYRLLS | 47 |
| WKASFTTFTVTKYWFYRL | 48 |
| GIWKASFTTFTVTKYWFY | 49 |
| FDGIWKASFTTFTVTKYW | 50 |
| HSFDGIWKASFTTFTVTK | 51 |
| FTTFTVTKYWFYRLLSALF | 52 |
| ASFTTFTVTKYWFYRLLSA | 53 |
| WKASFTTFTVTKYWFYRLL | 54 |
| GIWKASFTTFTVTKYWFYR | 55 |
| FDGIWKASFTTFTVTKYWF | 56 |
| HSFDGIWKASFTTFTVTKY | 57 |
| GTHSFDGIWKASFTTFTVT | 58 |
| FTTFTVTK | 59 |
| SFTTFTVT | 60 |
| FTTFTVTKY | 61 |
| SFTTFTVTK | 62 |
| FTTFTVTKYW | 63 |
| SFTTFTVTKY | 64 |
| ASFTTFTVTK | 65 |
| KASFTTFTVT | 66 |
| FTTFTVTKYWF | 67 |
| SFTTFTVTKYW | 68 |
| ASFTTFTVTKY | 69 |
| KASFTTFTVTK | 70 |
| WKASFTTFTVT | 71 |
| FTTFTVTKYWFY | 72 |
| SFTTFTVTKYWF | 73 |
| ASFTTFTVTKYW | 74 |
| WKASFTTFTVTK | 75 |
| FTTFTVTKYWFYR | 76 |
| ASFTTFTVTKYWF | 77 |
| WKASFTTFTVTKY | 78 |
| GIWKASFTTFTVT | 79 |
| SFTTFTVTKYWFYR | 80 |
| KASFTTFTVTKYWF | 81 |
| IWKASFTTFTVTKY | 82 |
| DGIWKASFTTFTVT | 83 |
| SFTTFTVTKYWFYRL | 84 |
| KASFTTFTVTKYWFY | 85 |
| IWKASFTTFTVTKYW | 86 |
| DGIWKASFTTFTVTK | 87 |
| FTTFTVTKYWFYRLLS | 88 |
| ASFTTFTVTKYWFYRL | 89 |
| WKASFTTFTVTKYWFY | 90 |
| GIWKASFTTFTVTKYW | 91 |
| FDGIWKASFTTFTVTK | 92 |
| FTTFTVTKYWFYRLLSA | 93 |
| ASFTTFTVTKYWFYRLL | 94 |
| WKASFTTFTVTKYWFYR | 95 |
| GIWKASFTTFTVTKYWF | 96 |
| FDGIWKASFTTFTVTKY | 97 |
| HSFDGIWKASFTTFTVT | 98 |
| SFTTFTVTKYWFYRLLSA | 99 |
| KASFTTFTVTKYWFYRLL | 100 |
| IWKASFTTFTVTKYWFYR | 101 |
| DGIWKASFTTFTVTKYWF | 102 |
| SFDGIWKASFTTFTVTKY | 103 |
| THSFDGIWKASFTTFTVT | 104 |
| SFTTFTVTKYWFYRLLSAL | 105 |
| KASFTTFTVTKYWFYRLLS | 106 |
| IWKASFTTFTVTKYWFYRL | 107 |
| DGIWKASFTTFTVTKYWFY | 108 |
| SFDGIWKASFTTFTVTKYW | 109 |
| THSFDGIWKASFTTFTVTK | 110 |

In some embodiments, the Cav-1 peptide or the modified Cav-1 peptide:
(a) consists of any one of the amino acid sequences of SEQ ID NOs: 1-110;
(b) comprises a core sequence of any one of the amino acid sequences of SEQ ID NOS: 1-110; or
(c) comprises a core sequence of any one of the amino acid sequences of SEQ ID NOs: 1-110, wherein the core sequence includes one or more amino acid substitutions, insertions, deletions, or chemical modifications.

In some embodiments, the Cav-1 peptide or the modified Cav-1 peptide comprises a core sequence of any one of the amino acid sequences of SEQ ID NO: 1-110. In some embodiments, the Cav-1 peptide or the modified Cav-1 peptide comprises a core sequence of any one of the amino acid sequences of SEQ ID NO: 1-110, wherein the core sequence includes one or more amino acid substitutions, insertions, deletions, or chemical modifications. In some embodiments, the core sequence is SEQ ID NO: 3. In some embodiments, the core sequence is SEQ ID NO: 6.

In some embodiments, the therapeutically active agent is a caveolin-1 peptide or derivative thereof. In some embodiments, the caveolin-1 peptide or derivative thereof comprises or consists of the amino acid sequence of any one of SEQ ID NOs: 1-110. In some embodiments, the caveolin-1 peptide or derivative thereof comprises an amino acid sequence with at least 40%, at least 50%, at least 60%, at least 70%, at least 80%, at least 85%, at least 90%, at least 95%, at least 96%, at least 97%, at least 98%, or at least 99% identity to any one of SEQ ID NOs: 1-110. In some embodiments, the caveolin-1 peptide or derivative thereof comprises the amino acid sequence of any one of SEQ ID NOs: 1-110 with one or more mutations relative thereto. For example, in some embodiments, the caveolin-1 peptide or derivative thereof comprises 1, 2, 3, 4, 5, 6, 7, 8, 9, 10, or more mutations relative to any one of SEQ ID NOs: 1-110. In some embodiments, the caveolin-1 peptide or derivative thereof comprises the amino acid sequence of any one of SEQ ID NOs: 1-110 with 1-5, 5-10, or 11-15, or more mutations. In some embodiments, the caveolin-1 peptide or derivative thereof comprises an additional 1-5 amino acids at either the N- or C-terminus or at both termini of any one of SEQ ID NOs: 1-110.

In some embodiments, the therapeutically active agent is a caveolin-1 peptide or derivative thereof. In some embodiments, the caveolin-1 peptide or derivative thereof comprises or consists of the amino acid sequence of SEQ ID NO: 1. In some embodiments, the caveolin-1 peptide or derivative thereof comprises an amino acid sequence with at least 40%, at least 50%, at least 60%, at least 70%, at least 80%, at least 85%, at least 90%, at least 95%, at least 96%, at least 97%, at least 98%, or at least 99% identity to SEQ ID NO: 1. In some embodiments, the caveolin-1 peptide or derivative thereof comprises the amino acid sequence of SEQ ID NO: 1 with one or more mutations relative thereto. For example, in some embodiments, the caveolin-1 peptide or derivative thereof comprises 1, 2, 3, 4, 5, 6, 7, 8, 9, 10, or more mutations relative to SEQ ID NO: 1. In some embodiments, the caveolin-1 peptide or derivative thereof comprises the amino acid sequence of SEQ ID NO: 1 with 1-5, 5-10, 11-5, 15-20, 10-25, 25-30, or more than 30 mutations.

In some embodiments, the therapeutically active agent is a caveolin-1 peptide or derivative thereof. In some embodiments, the caveolin-1 peptide or derivative thereof comprises or consists of the amino acid sequence of SEQ ID NO: 2. In some embodiments, the caveolin-1 peptide or derivative thereof comprises an amino acid sequence with at least 40%, at least 50%, at least 60%, at least 70%, at least 80%, at least 85%, at least 90%, at least 91%, at least 92%, at least 93%, at least 94%, or at least 95% identity to SEQ ID NO: 2. In some embodiments, the caveolin-1 peptide or derivative thereof comprises the amino acid sequence of SEQ ID NO: 2 with one or more mutations relative thereto. For example, in some embodiments, the caveolin-1 peptide or derivative thereof comprises 1, 2, 3, 4, 5, 6, 7, 8, 9, 10, or more mutations relative to SEQ ID NO: 2. In some embodiments, the caveolin-1 peptide or derivative thereof comprises the amino acid sequence of SEQ ID NO: 2 with 1-5, 5-10, or 11-15, or more mutations. In some embodiments, the caveolin-1 peptide or derivative thereof comprises an additional 1-5 amino acids at either the N- or C-terminus or at both termini of SEQ ID NO: 2.

In some embodiments, the therapeutically active agent is a caveolin-1 peptide or derivative thereof. In some embodiments, the caveolin-1 peptide or derivative thereof comprises or consists of the amino acid sequence of SEQ ID NO: 3. In some embodiments, the caveolin-1 peptide or derivative thereof comprises an amino acid sequence with at least 40%, at least 50%, at least 60%, at least 70%, at least 80%, or at least 85% identity to SEQ ID NO: 3. In some embodiments, the caveolin-1 peptide or derivative thereof comprises the amino acid sequence of SEQ ID NO: 3 with one or more mutations relative thereto. For example, in some embodiments, the caveolin-1 peptide or derivative thereof comprises 1, 2, 3, 4, or 5 mutations relative to SEQ ID NO: 3. In some embodiments, the caveolin-1 peptide or derivative thereof comprises an additional 1-5 amino acids at either the N- or C-terminus or at both termini of SEQ ID NO: 3.

In some embodiments, the therapeutically active agent is a caveolin-1 peptide or derivative thereof. In some embodiments, the caveolin-1 peptide or derivative thereof comprises or consists of the amino acid sequence of SEQ ID NO: 4. In some embodiments, the caveolin-1 peptide or derivative thereof comprises an amino acid sequence with at least 40%, at least 50%, at least 60%, at least 70%, at least 80%, at least 85%, at least 90%, at least 91%, at least 92%, at least 93%, at least 94%, or at least 95% identity to SEQ ID NO: 4. In some embodiments, the caveolin-1 peptide or derivative thereof comprises the amino acid sequence of SEQ ID NO: 4 with one or more mutations relative thereto. For example, in some embodiments, the caveolin-1 peptide or derivative thereof comprises 1, 2, 3, 4, 5, 6, 7, 8, 9, 10, or more mutations relative to SEQ ID NO: 4. In some embodiments, the caveolin-1 peptide or derivative thereof comprises the amino acid sequence of SEQ ID NO: 4 with 1-5, 5-10, or 11-15, or more mutations. In some embodiments, the caveolin-1 peptide or derivative thereof comprises an additional 1-5 amino acids at either the N- or C-terminus or at both termini of SEQ ID NO: 4.

In some embodiments, the caveolin-1 peptide or derivative thereof comprises at least one non-standard amino acid. In some embodiments, the caveolin-1 peptide or derivative thereof comprises two or more non-standard amino acids. In some embodiments, the caveolin-1 peptide or derivative thereof comprises four or more non-standard amino acids. In some embodiments, the non-standard amino acid is ornithine. In some embodiments, the non-standard amino acid is D-alanine. In some embodiments, the caveolin-1 peptide or derivative thereof comprises one or more non-standard amino acids, wherein the non-standard amino acid is ornithine.

In some embodiments, the caveolin-1 peptide or derivative thereof comprises L-amino acids. In some embodiments, the caveolin-1 peptide or derivative thereof comprises D-amino acids. In some embodiments, the caveolin-1 peptide or derivative thereof comprises L-amino acids and D-amino acids. In some embodiments, the caveolin-1 peptide or derivative thereof comprises one or more D-amino acids, wherein the D-amino acid is D-alanine.

In some embodiments, the caveolin-1 peptide or derivative thereof comprises N- or C-terminal modifications. In some embodiments, the caveolin-1 peptide or derivative thereof comprises an N-terminal modification. In some embodiments, the caveolin-1 peptide or derivative thereof comprises a C-terminal modification. In some embodiments, the N-terminal modification is acylation. In some embodiments, the C-terminal modification is amidation.

In some embodiments, the caveolin-1 peptide or derivative thereof comprises a cell-penetrating peptide (CPP). Examples of CPPs are shown in Table 3 below.

TABLE 3
Exemplary Cell Penetrating Peptides

| | SEQ ID |
|---|---|
| Amino Acid Sequence | NO. |
| GRKKRRQRRRPPQ | 111 |
| RQIKIWFQNRRMKWKK | 112 |
| GIGAVLKVLTTGLPALISWIKRKRQQ | 113 |

In some embodiments, the caveolin-1 peptide or derivative thereof is conjugated to a heterologous polypeptide segment or polymer, such as polyethylene glycol (PEG). Caveolin-1 peptide or derivatives thereof may be linked to PEG to increase serum persistence.

In some embodiments, the therapeutically active agent is a peptidomimetic compound that mimics the biological effects of the native caveolin-1 peptide or derivative thereof. A peptidomimetic agent may be an unnatural peptide or a non-peptide agent that recreates the stereospatial properties of the binding elements of the native caveolin-1 peptide or derivative thereof such that it has the binding activity and biological activity of the native caveolin-1 peptide or derivative thereof.

### Other Types of Therapeutically Active Agents

In some embodiments, the therapeutically active agent is an agent that reduces or inhibits the activity of transforming growth factor-β (TGF-β). Examples of therapeutically active agents that reduce or inhibit the activity of TGF-β include, but are not limited to, GC-1008 (fresolimumab, Genzyme/MedImmune), lerdelimumab (CAT-152; Trabio, Cambridge Antibody), metelimumab (CAT-192, Cambridge Antibody), LY-2157299 (Eli Lilly), and ACU-HTR-028 (Opko Health), as well as antibodies that target one or more TGF-β isoforms, and inhibitors of TGF-β receptor kinases TGFBR1 (ALK5) and TGFBR2.

In some embodiments, the therapeutically active agent is pirfenidone (also referred to as esbriet). Pirfenidone is a small molecule that has anti-fibrotic and anti-inflammatory properties. Pirfenidone has been shown to downregulate growth factors (e.g., TGF-β), procollagen I and II, and inflammatory mediators (e.g., TNF-α and IL-β).

In some embodiments, the therapeutically active agent is an endothelin receptor antagonist. In some embodiments, the endothelin receptor antagonist targets both endothelin receptor A and endothelin receptor B. In some embodiments, the endothelin receptor antagonist targets endothelin receptor A. Examples of endothelin receptor antagonists include, but are not limited to, ambrisentan, avosentan, bosentan, clazosentan, darusenta, BQ-153, FR-139317, L-744453, macitentan, PD-145065, PD-156252, PD163610, PS-433540, S-0139, sitaxentan sodium, TBC-3711, and zibotentan.

In some embodiments, the therapeutically active agent is an agent that reduces or inhibits the activity of connective tissue growth factor (CTGF). Examples of therapeutically active agents that reduce or inhibit the activity of CTGF include, but are not limited to, FG-3019 and FibroGen, CTGF-neutralizing antibodies, and matrix metalloproteinase (MMP) inhibitors such as MMP-12, PUP-1 and tigapotide triflutate.

In some embodiments, the therapeutically active agent is an agent that reduces or inhibits the activity of epidermal growth factor receptor (EGFR). Examples of therapeutically active agents that reduce or inhibit the activity of EGFR include, but are not limited to, erlotinib, gefitinib, BMS-690514, cetuximab, antibodies targeting EGFR, inhibitors of EGFR kinase, and modulators of post-receptor signaling pathways.

In some embodiments, the therapeutically active agent is an agent that reduces or inhibits the activity of platelet derived growth factor (PDGF). Examples of therapeutically active agents that reduce or inhibit the activity of PDGF include, but are not limited to, imatinib mesylate (Novartis), PDGF neutralizing antibodies, antibodies targeting PDGF receptor (PDGFR), inhibitors of PDGFR kinase activity, and post-receptor signaling pathways.

In some embodiments, the therapeutically active agent is an agent that reduces or inhibits the activity of vascular endothelial growth factor (VEGF). Examples of therapeutically active agents that reduce or inhibit the activity of VEGF include, but are not limited to, axitinib, bevacizumab, BIBF-1120, CDP-791, CT-322, IMC-18F1, PTC-299, ramucirumab, VEGF-neutralizing antibodies, antibodies targeting the VEGF receptor 1 (VEGFR1, Flt-1) and VEGF receptor 2 (VEGFR2, KDR), the soluble form of VEGFR1 (sFlt) and derivatives thereof which neutralize VEGF, and inhibitors of VEGF receptor kinase activity.

In some embodiments, the therapeutically active agent is an agent that inhibits of multiple receptor kinases. For example, BIBF-1120 (also referred to herein as nintedanib) is a therapeutically active agent that inhibits receptor kinases for vascular endothelial growth factor, fibroblast growth factor, and platelet derived growth factor.

In some embodiments, the therapeutically active agent is an agent that interferes with integrin function. Examples of therapeutically active agents that interfere with integrin function include, but are not limited to, STX-100, IMGN-388, and integrin-targeted antibodies.

In some embodiments, the therapeutically active agent is an agent that interferes with the pro-fibrotic activities of IL-4 and IL-13. Examples of therapeutically active agents that interfere with the pro-fibrotic activities of IL-4 and IL-13 include, but are not limited to, AER-001, AMG-317, APG-201, sIL-4Rα, AER-001, AMG-317, anrukinzumab, CAT-354, cintredekin besudotox, MK-6105, QAX-576, SB-313, SL-102, TNX-650, tralokinumab, lebrikizumab, SAR156597, antibodies that target IL-4 receptor or IL-13 receptor, the soluble form of IL-4 receptor or derivatives thereof that bind and neutralize both IL-4 and IL-13, chimeric proteins including all or part of IL-13, and inhibitors of the JAK-STAT kinase pathway.

In some embodiments, the therapeutically active agent is an agent that inhibits CCL-2, such as an anti-CCL2 antibody (e.g., CNT0888).

In some embodiments, the therapeutically active agent is an agent that interferes with epithelial mesenchymal transition, e.g., inhibitors of mTOR (including, but not limited to AP-23573 and rapamycin).

In some embodiments, the therapeutically active agent is an agent that reduces levels of copper, e.g., tetrathiomolybdate.

In some embodiments, the therapeutically active agent is an agent that reduces oxidative stress. Examples of therapeutically active agents that reduce oxidative stress include, but are not limited to, N-acetyl cysteine, tetrathiomolybdate, and interferon gamma.

In some embodiments, the therapeutically active agent is an inhibitor of phosphodiesterase 4 (e.g., Roflumilast); an inhibitor of phosphodiesterase 5 (e.g., mirodenafil, PF-4480682, sildenafil citrate, SLx-2101, tadalafil, udenafil, UK-369003, vardenafil, and zaprinast), or a modifier of the arachidonic acid pathway, including cyclooxygenase and 5-lipoxegenase inhibitors (e.g., zileuton).

In some embodiments, the therapeutically active agent reduces tissue remodeling or fibrosis. Examples of therapeutically active agents that reduce tissue remodeling or fibrosis include, but are not limited to, prolyl hydrolase inhibitors (e.g., 1016548, CG-0089, FG-2216, FG-4497, FG-5615, FG-6513, fibrostatin A (Takeda), lufironil, P-1894B, and safironil), peroxisome proliferator-activated receptor (PPAR)-gamma agonists (e.g., pioglitazone and rosiglitazone), carbon monoxide, and doxycycline.

In some embodiments, the therapeutically active agent is an agent that has antioxidant, immunosuppressant and/or anti-inflammatory activities such as N-acetylcysteine.

In some embodiments, the therapeutically active agent is an agent that inhibits somatostatin receptors, such as somatostatin analogs (e.g, SOM230 and octreotide).

In some embodiments, the therapeutically active agent is an agent that has anti-angiogenic, immunomodulatory, and/or anti-inflammatory activities such as thalidomide or minocycline.

In some embodiments, the therapeutically active agent is an agent that inhibits the enzyme lysyl oxidase-like 2 (LOXL2), such as an anti-LOXL2 antibody (e.g., GS-6624).

In some embodiments, the therapeutically active agent is an agent that targets the renin-angiotensin system (e.g., losartan).

Other examples of therapeutically active agents for use in the present disclosure are described, e.g., Rafii et al., J. Thorac Dis, 2013; 5 (1): 48-73; US Pat. Pub. No. 2019/0062836A1; US Pat. Pub. No. 2012/0014917A1; WO 2014/145389A1; and WO 2020/055812 A1, which are hereby incorporated by reference in their entireties.

Methods of Treatment with the Therapeutically Active Agents

The present disclosure provides biomarkers associated with a therapeutically active agent in a subject with a disease or disorder (e.g., interstitial lung disease). In some embodiments, one or more therapeutically active agents can be administered in any of the methods disclosed herein.

In some embodiments, the therapeutically active agents described herein are administered in vitro to a biological sample. In some embodiments, the therapeutically active agent is administered in vitro to a biological sample obtained from a subject with a disease or disorder. In some embodiments, the therapeutically active agent is administered in vitro to a biological sample obtained from a subject with interstitial lung disease. In some embodiments, the therapeutically active agent is administered in vitro to a biological sample obtained from a subject with idiopathic pulmonary fibrosis. In some embodiments, the biological sample is fibroblasts isolated from a subject with idiopathic pulmonary fibrosis. In some embodiments, the biological sample is lung tissue isolated from a subject with idiopathic pulmonary fibrosis. In some embodiments, the biological sample is basal-like cells isolated from a subject with idiopathic pulmonary fibrosis. In some embodiments, the therapeutically active agent is a caveolin-1 peptide or derivative thereof (e.g., CSP-7).

In some embodiments, the therapeutically active agent is administered in vivo to a subject with a disease or disorder. In some embodiments, the therapeutically active agent is administered in vivo to a subject with interstitial lung disease. In some embodiments, the therapeutically active agent is administered in vivo to a subject with idiopathic pulmonary fibrosis. In some embodiments, a caveolin-1 peptide or derivative thereof (e.g., CSP-7) is administered in vivo to a subject with idiopathic pulmonary fibrosis.

In some embodiments, the therapeutically active agent (e.g., a caveolin-1 peptide or derivative thereof) comprises at least one pharmaceutically acceptable carrier or excipient. In some embodiments, the therapeutically active agent comprising at least one pharmaceutically acceptable carrier or excipient is administered to a biological sample in vitro. In some embodiments, the therapeutically active agent comprising at least one pharmaceutically acceptable carrier or excipient is administered to a subject with a disease or disorder in vivo.

In some embodiments, the therapeutically active agent (e.g., a caveolin-1 peptide or derivative thereof) is administered in the absence of a pharmaceutically acceptable carrier or excipient. In some embodiments, the therapeutically active agent is administered to a biological sample in vitro in the absence of a pharmaceutically acceptable carrier or excipient. In some embodiments, the therapeutically active agent is administered to a subject with a disease or disorder in vivo in the absence of a pharmaceutically acceptable carrier or excipient.

In some embodiments, the therapeutically active agent is administered orally, parenterally, sublingually, transdermally, rectally, transmucosally, topically, via inhalation, via buccal administration, intrapleurally, intravenously, intraarterially, intragastrically, intraperitoneally, subcutaneously, intramuscularly, intranasally intrathecally, and intraarticularly, or combinations thereof, to a subject with a disease or disorder, e.g., interstitial lung disease.

In some embodiments, the caveolin-1 peptide or derivative thereof is administered orally, parenterally, sublingually, transdermally, rectally, transmucosally, topically, via inhalation, via buccal administration, intrapleurally, intravenously, intraarterially, intragastrically, intraperitoneally, subcutaneously, intramuscularly, intranasally intrathecally, and intraarticularly, or combinations thereof, to a subject with idiopathic pulmonary fibrosis. In some embodiments, the caveolin-1 peptide or derivative thereof is administered intravenously to a subject with idiopathic pulmonary fibrosis. In some embodiments, the caveolin-1 peptide or derivative thereof is administered via inhalation to a subject with idiopathic pulmonary fibrosis. In some embodiments, the caveolin-1 peptide or derivative thereof is administered intranasally to a subject with idiopathic pulmonary fibrosis. In some embodiments, the caveolin-1 peptide or derivative thereof is CSP-7. In some embodiments, the caveolin-1 peptide or derivative thereof is Var55.

In some embodiments, the therapeutically active agent is administered to a subject with a disease or disorder once per day, twice per day, three times per day, once per week, twice per week, three times per week, five times per week, once every two weeks, one every three weeks, once per month, once every two months, once every three months, or once every six months. In some embodiments, the therapeutically active agent is administered to a subject with interstitial lung disease once per day, twice per day, three times per day, once per week, twice per week, three times per week, five times per week, once every two weeks, one every three weeks, once per month, once every two months, once every three months, or once every six months. In some embodiments, the therapeutically active agent is administered to a subject with idiopathic pulmonary fibrosis once per day, twice per day, three times per day, once per week, twice per week, three times per week, five times per week, once every two weeks, one every three weeks, once per month, once every two months, once every three months, or once every six months.

In some embodiments, the caveolin-1 peptide or derivative thereof is administered to a subject with a disease or disorder once per day, twice per day, three times per day, once per week, twice per week, three times per week, five times per week, once every two weeks, one every three weeks, once per month, once every two months, once every three months, or once every six months. In some embodiments, the caveolin-1 peptide or derivative thereof is administered to a subject with interstitial lung disease once per day, twice per day, three times per day, once per week, twice per week, three times per week, five times per week, once every two weeks, one every three weeks, once per month, once every two months, once every three months, or once every six months. In some embodiments, the caveolin-1 peptide or derivative thereof is administered to a subject with idiopathic pulmonary fibrosis once per day, twice per day, three times per day, once per week, twice per week, three times per week, five times per week, once every two weeks, one every three weeks, once per month, once every two months, once every three months, or once every six months. In some embodiments, the caveolin-1 peptide or derivative thereof is CSP-7. In some embodiments, the caveolin-1 peptide or derivative thereof is Var55.

In some embodiments, the therapeutically active agent is administered to a subject with a disease or disorder at a dose of about 0.01 mg/kg to about 250 mg/kg. In some embodiments, the therapeutically active agent is administered to a subject with a disease or disorder at a dose of about 0.01 mg/kg to about 50 mg/kg. In some embodiments, the therapeutically active agent is administered to a subject with a disease or disorder at a dose of about 0.05 mg/kg to about 50 mg/kg. In some embodiments, the therapeutically active agent is administered to a subject with a disease or disorder at a dose of about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 75 mg/kg, about 100 mg/kg, about 125 mg/kg, about 150 mg/kg, about 175 mg/kg, about 200 mg/kg, about 225 mg/kg, about 250 mg/kg, or more.

In some embodiments, the caveolin-1 peptide or derivative thereof is administered to a subject with idiopathic pulmonary fibrosis at a dose of about 0.01 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 75 mg/kg, about 100 mg/kg, about 125 mg/kg, about 150 mg/kg, about 175 mg/kg, about 200 mg/kg, about 225 mg/kg, about 250 mg/kg, or more. In some embodiments, the caveolin-1 peptide or derivative thereof is CSP-7. In some embodiments, the caveolin-1 peptide or derivative thereof is Var55.

### Biomarkers for Caveolin-1 Peptide Therapy

The present disclosure provides biomarkers associated with a therapeutically active agent in a subject with a disease or disorder. In some embodiments, the biomarkers are associated with a caveolin-1 peptide or derivative thereof in a subject with interstitial lung disease, e.g., idiopathic pulmonary fibrosis.

Exemplary biomarkers for caveolin-1 therapy in subjects with interstitial lung disease are shown in Table 4 below.

TABLE 4
Exemplary Biomarkers for Caveolin-1 Peptide Therapy

| | | Other | Protein | Gene |
|---|---|---|---|---|
| Protein Name | Gene | Aliases | ID | ID |
| Myeloid Derived Growth | MYDGF | C19orf10 | Q969H8 | 56005 |
| Factor (MYDGF) | | | | |
| Advanced Glycosylation | AGER | RAGE | Q15109 | 177 |
| End-Product Specific | | | | |
| Receptor (AGER) | | | | |
| Mothers against | SMAD2 | MADH2 | Q15796 | 4087 |
| decapentaplegic homolog | | MADR2 | | |
| 2 (SMAD2) | | | | |
| Mothers against | SMAD3 | MADH3 | P84022 | 4088 |
| decapentaplegic homolog | | | | |
| 3 (SMAD3) | | | | |
| Platelet-derived growth | PDGFRB | PDGFR1 | P09619 | 5159 |
| factor receptor beta | | | | |
| Profilin 1 (PROF1) | PFN1 | ALS18 | P07737 | 5216 |
| Calmodulin-2 | CALM2 | CAM2 | P0DP24 | 801 |
| | | CAMB | | 805 |
| | | PHKD | | 808 |
| Calreticulin | CALR | CRTC | P27797 | 811 |
| | | CRP55 | | |
| | | Calregulin | | |
| | | ERp60 | | |
| | | HACBP | | |
| | | grp60 | | |
| Peptidyl-prolyl cis-trans | PPIA | CYPA | P62937 | 5478 |
| isomerase A | | PPIaseA | | |
| Eukaryotic translation | EIF5A | eIF-5A | P63241 | 1984 |
| initiation factor 5A | | eIF-4D | | |
| (EIF5A) | | | | |
| Galectin-1 | LGALS1 | Gal-1 | P09382 | 3956 |
| | | HLBP14 | | |
| | | HBL | | |
| | | HPL | | |
| Annexin A5 | ANXA5 | ANX5 | P08758 | 308 |
| | | ENX2 | | |
| | | PP4 | | |
| Fatty Acid Binding | FABP5 | E-FABP | Q01469 | 2171 |
| Protein 5 | | | | |
| Glutathione S-transferase | GSTP1 | FAEES3 | P09211 | 2950 |
| P | | GST3 | | |
| Alpha-enolase | ENO1 | ENO1L1 | P06733 | 2023 |
| | | MBPB1 | | |
| | | MPB1 | | |
| 14-3-3 protein gamma | YWHAG | 1433G | P61981 | 7532 |
| Galectin 7 | LGALS7 | GAL-7 | P47929 | 3963 |
| | | PIG1 | | |
| Serine/threonine-protein | mTOR | FRAP | P42345 | 2475 |
| kinase mTOR | | RAFT1 | | |
| | | RAPT1 | | |
| Interleukin-11 | IL11 | AGIF | P20809 | 3589 |
| 72 kDa type IV | MMP2 | MMP2 | P08253 | 4313 |
| collagenase | | Gelatinase A | | |
| Platelet basic protein | PPBP | CXCL7 | P02775 | 5473 |
| | | LDGF | | |
| | | MDGF | | |
| Scavenger receptor | CD163 | CD163 | Q86VB7 | 9332 |
| cysteine-rich type 1 | | M130 | | |
| protein M130 | | | | |

In some embodiments, the biomarker is selected from the group consisting of MYDGF, soluble RAGE, phosphorylated SMAD2 (pSMAD2), pSMAD3, PDGFRβ, phosphorylated PDGFRβ (pPDGFRβ), PROF1, CALM2, CALR, PPIA, EIF5A, LGALS1, ANXA5, FABP5, GSTP1, ENO1, YWHAG, LGALS7, IL-11, MMP-2, CXCL7, soluble CD163 (sCD163), or phosphorylated mTOR (p-mTOR). In some embodiments, the biomarker is selected from the group consisting of MYDGF, soluble RAGE, phosphorylated SMAD2 (pSMAD2), pSMAD3, PDGFB, PROF1, CALM2, CALR, PPIA, EIF5A, LGALS1, ANXA5, FABP5, GSTP1, ENO1, and YWHAG. In some embodiments, the biomarker is MYDGF. In some embodiments, the biomarker is soluble RAGE. In some embodiments, the biomarker is pSMAD2 and/or pSMAD3. In some embodiments, the biomarker is PDGFRβ or pPDGFRβ. In some embodiments, the biomarker is PROF1. In some embodiments, the biomarker is CALM2. In some embodiments, the biomarker is CALR. In some embodiments, the biomarker is PPIA. In some embodiments, the biomarker is EIF5A. In some embodiments, the biomarker is LGALS1. In some embodiments, the biomarker is ANXA5. In some embodiments, the biomarker is FABP5. In some embodiments, the biomarker is GSTP1. In some embodiments, the biomarker is ENO1. In some embodiments, the biomarker is YWHAG. In some embodiments, the biomarker is LGALS7. In some embodiments, the biomarker is p-mTOR. In some embodiments, the biomarker is IL-11. In some embodiments, the biomarker is MMP-2. In some embodiments, the biomarker is CXCL7. In some embodiments, the biomarker is sCD163.

MYDGF is a paracrine-acting protein secreted primarily by monocytes and macrophages. MYDGF is also expressed by eosinophils, bone marrow cells, synoviocytes, cholangiocarcinoma cells, and hepatocellular carcinoma cells. MYDGF promotes tissue repair and heart function after myocardial infarction, thereby protecting against cardiac injury. Cytoprotection by MYDGF is associated with phosphorylation of AKT and inhibition of apoptosis and MYDGF has also been shown to promote proliferation of human coronary artery endothelial cells. Mice deficient in MYDGF exhibited larger infarct scars and decreased cell proliferation and angiogenesis at the infarct border zone compared to wild-type mice. See, Korf-Klingebiel et al., Nat. Med. 21:140-149 (2015).

Advanced Glycosylation End-Product Specific Receptor (RAGE) is a member of the immunoglobulin superfamily of cell surface receptors. It is a multi-ligand receptor that binds AGE, as well as other molecules, to regulate inflammation, oxidative stress, homeostasis, and development. RAGE is abundantly expressed in the normal lung, especially in type 1 pneumocytes. Previous studies have shown that RAGE expression in lung tissue is reduced in IPF when compared with controls, and functional polymorphism of the RAGE gene is associated with risk of IPF. See, Yamaguchi et al., Respirology, 2017; 22:965-71; Ishikawa et al., Respir Res, 2010; 11:123; Manichaikul et al., Ann Am Thorac Soc, 2017; 14:628-35; Konishi et al., Am J Respir Crit Care Med, 2009; 180:167-75; and Yamaguchi et al., Respir Res, 2020, 21:145.

Soluble forms of RAGE lack a transmembrane domain but have an extracellular ligand-binding domain. There are several types of soluble RAGE, including endogenous secretory RAGE (esRAGE), which is a splice variant of RAGE, as well as a shedded form of RAGE derived from cell-surface RAGE. Soluble RAGE has anti-inflammatory properties and acts as a decoy receptor by neutralizing RAGE-ligands. Previous studies have shown through proteomic analysis that soluble RAGE expression was reduced in the lung tissue of subjects with IPF and not in chronic obstructive pulmonary disease (COPD), suggesting that soluble RAGE may be related to the pathophysiology of IPF (see, Ishikawa et al., Respir Res, 2010, 11:123). Other studies have shown a reduction of soluble RAGE in lung tissue, serum, and BAL of subjects with IPF (see, Yamaguchi et al., Respiratory Research, 2020; 21:145).

Mothers against decapentaplegic homolog (SMAD) proteins are signal transducers and transcriptional modulators that mediate multiple signaling pathways. Both SMAD2 and SMAD3 mediate TGF-β signaling, thereby regulating several important cellular processes, including cell proliferation, apoptosis, and differentiation. SMAD2 and SMAD3 are recruited to the TGF-β receptor through its interaction with the SMAD anchor for receptor activation (SARA) protein and are then phosphorylated by the TGF-β receptor. The phosphorylation induces the dissociation of SMAD2/3 with SARA and the association with the family member SMAD4. The association with SMAD4 is important for the translocation of this protein into the nucleus, where it regulates a number of target genes involved in matrix expression, proliferation, and cell differentiation. See, Gauldie et al., Proc Am Thorac Soc, 2006; 3 (8): 696-702.

Platelet-derived growth factor B (PDGFB) belongs to a family of proteins comprised of other PDGFs and vascular endothelial growth factors (VEGF). The PDGFB preproprotein is proteolytically processed to generate platelet-derived growth factor subunit B, which can homodimerize, or alternatively, heterodimerize with the related platelet-derived growth factor subunit A. These proteins bind and activate PDGF receptor tyrosine kinases, which play a role in a wide range of developmental processes. PDGF signals through platelet-derived growth factor receptor beta (PDGFRβ). PDGF signaling has been implicated in the pathogenesis of pulmonary fibrosis. A number of fibrogenic mediators such as TGF-β, IL-1, TNF-α, bFGF, and thrombin exhibit PDGF-dependent profibrotic activities. Inhibition of PDGFRβ ameliorated bleomycin-induced pulmonary fibrosis in mice. See, Abdollahi et al., JEM, 2005 201 (6): 925-935; and Kishi et al., PLOS One, 2018, 13 (12): e0209786).

Profilin-1 (PROF1) is a member of the prolifin family of small actin-binding proteins. Profilin-1 plays an important role in actin dynamics by regulating actin polymerization in response to extracellular signals. Prolifin-1 affects the structure of the cytoskeleton. Deletion of profilin-1 is associated with Miller-Dieker syndrome and may also play a role in Huntington disease.

Calmodulin (CALM2) mediates the control of a large number of enzymes, ion channels, aquaporins, and other proteins through calcium-binding. A number of protein kinases and phosphatases are also stimulated by the calmodulin-calcium complex. Together with CCP110 and centrin, calmodulin is involved in a genetic pathway that regulates the centrosome cycle and progression through cytokinesis (see, Tsang et al., Mol Biol Cell, 2006, 17:3423-3434).

Calreticulin (CALR) is a calcium-binding chaperone that promotes folding, oligomeric assembly, and quality control in the endoplasmic reticulum (ER) via the calreticulin/calnexin cycle. This lectin interacts transiently with almost all of the monoglucosylated glycoproteins that are synthesized in the ER. Calreticulin also interacts with the DNA-binding domain of NR3C1 to mediate its nuclear export.

Peptidyl-prolyl cis-trans isomerase A (PPIA) belongs to the peptidyl-prolyl cis-trans isomerase (PPIase) family. PPIases catalyze the cis-trans isomerization of proline imidic peptide bonds in oligopeptides and accelerate the folding of proteins. PPIA is also a cyclosporin binding-protein and may play a role in cyclosporine A-mediated immunosuppression.

Eukaryotic translation initiation factor 5A (EIF5A) is an mRNA-binding protein involved in translation elongation. EIF5A also plays an important role in mRNA turnover. EIF5A is involved in actin dynamics and cell cycle progression, and regulates p53/TP53-dependent apoptosis and TNFα-mediated apoptosis.

Galectin-1 (LGALS1) binds beta-galactoside and a wide array of complex carbohydrates. Galectin-1 plays a role in regulating apoptosis, cell proliferation, and cell differentiation. Galactin-1 has been previously shown to inhibit CD45 phosphatase activity and the dephosphorylation of Lyn kinase. Galectin-1 is a strong inducer of T cell apoptosis.

Annexin A5 (ANXA5) belongs to the annexin family of calcium-dependent phospholipid binding proteins. Annexin A5 is a phospholipase A2 and protein kinase C inhibitory protein with calcium channel activity and has also been implicated in cellular signal transduction, inflammation, growth and differentiation.

Fatty Acid Binding Protein 5 (FABP5) belongs to a family of small, highly conserved, cytoplasmic proteins that bind long-chain fatty acids and other hydrophobic ligands. FABPs may play roles in fatty acid uptake, transport, and metabolism. In addition to the cytosolic transport, FABP5 selectively delivers specific fatty acids from the cytosol to the nucleus, wherein they activate nuclear receptors. FABP5 also delivers retinoic acid to the nuclear receptor peroxisome proliferator-activated receptor delta, which promotes proliferation and survival.

Glutathione S-transferase P (GSTP1) is a member of the GST superfamily that is prevalently expressed in mammals. GSTP functions to deprotonate glutathione allowing formation of thioether bonds with electrophilic substrates. In addition to catalytic detoxification, other properties of GSTP1 include chaperone functions, regulation of nitric oxide pathways, regulation of a variety of kinase signaling pathways, and participation in the forward reaction of protein S-glutathionylation. See, Zhang et al., Advances in Cancer Res, 2014, 122:143-175,

Alpha-enolase 1 (ENO1) is a glycolytic enzyme that catalyzes the conversion of 2-phosphoglycerate to phosphoenolpyruvate. In addition to glycolysis, ENO1 is involved in various cellular processes, including plasminogen binding, maintenance of mitochondrial membrane stability, RNA chaperone activity and signal transduction (see, Didiasova et al., Front Cell Dev Biol, 2019).

14-3-3 protein gamma (YWHAG) is an adapter protein implicated in the regulation of a large spectrum of both general and specialized signaling pathways. 14-3-3 protein gamma binds to a large number of protein partners usually by recognition of a phosphoserine or phosphothreonine motif and binding generally results in the modulation of the activity of the binding partner.

Galectin-7 (LGALS7) belongs to a family of beta-galactoside-binding proteins implicated in modulating cell-cell and cell-matrix interactions for normal growth control. Galectin-7 has many functions including roles in cell adhesion, migration, and immune cell regulation.

mTOR is a serine/threonine-protein kinase and central regulator of cellular metabolism, growth, proliferation, and survival. mTOR is the catalytic subunit of two distinct protein complexes, mTORC1 and mTORC2 and is involved in the regulation of at least 800 different proteins. Phosphorylation of mTOR enhances its activity thereby promoting activation of downstream signaling pathways.

Interleukin-11 (IL-11) is a pleiotropic cytokine that belongs to the IL-6 family of cytokines. Noncanonical IL-11 signaling drives pathologies common to all fibro-inflammatory disease, including myofibroblast activation, parenchymal cell dysfunction, and inflammation, while also inhibiting tissue regeneration (see, Cook and Schafer, Annual Review of Medicine, 2020; 71:263-276).

MMP-2 belongs to the matrix metalloproteinase family, which are zinc-dependent enzymes capable of cleaving components of the extracellular matrix, as well as signal transduction molecules. MMP-2 is considered the most ubiquitous metalloproteinase in this enzyme family and is involved in many diverse cellular functions, including the regulation of blood vessel formation and remodeling, and tissue repair and regeneration.

CXCL7 belongs to the CXC chemokine family and is a potent chemoattractant and activator of neutrophils. It has been shown to stimulate various cellular processes including DNA synthesis, mitosis, glycolysis, intracellular cAMP accumulation, prostaglandin E2 secretion, and synthesis of hyaluronic acid and sulfated glycosaminoglycan.

CD163 is a member of the scavenger receptor cysteine-rich (SRCR) superfamily and is primarily expressed on monocytes and macrophages. It functions as an acute-phase regulator receptor involved in the clearance and endocytosis of hemoglobin/haptoglobin complexes by macrophages, and may thereby protect tissues from free hemoglobin-mediated oxidative damage. A soluble form of CD163 (sCD163) is released from the cell surface by proteolysis after oxidative stress or inflammatory stimuli. sCD163 is considered a biomarker for macrophage activation in several inflammatory diseases, such as sepsis, inflammatory bowel disease, and influenza-associated encephalopathy. See, Etzerodt et al., Scientific Reports, 2017; 7:40286.

In some embodiments, the expression level of a biomarker is increased in a biological sample treated with a therapeutically active agent (e.g., a caveolin-1 peptide or derivative thereof) compared to a control sample. In some embodiments, the expression level of the biomarker in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of the biomarker in a biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of the biomarker in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis. In some embodiments, the biomarker is selected from the group consisting of MYDGF, soluble RAGE, phosphorylated SMAD2 (pSMAD2), pSMAD3, PDGFRβ, pPDGFRβ, PROF1, CALM2, CALR, PPIA, EIF5A, LGALS1, ANXA5, FABP5, GSTP1, ENO1, YWHAG, LGALS7, IL-11, MMP-2, CXCL7, sCD163, and p-mTOR.

In some embodiments, the expression level of MYDGF is increased in a biological sample treated with a therapeutically active agent (e.g., a caveolin-1 peptide or derivative thereof) compared to a control sample. In some embodiments, the expression level of MYDGF in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of MYDGF in the biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of MYDGF in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of soluble RAGE is increased in a biological sample treated with a therapeutically active agent (e.g., a caveolin-1 peptide or derivative thereof) compared to a control sample. In some embodiments, the expression level of soluble RAGE in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of soluble RAGE in the biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of soluble RAGE in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of prolifin is increased in a biological sample treated with a therapeutically active agent (e.g., a caveolin-1 peptide or derivative thereof) compared to a control sample. In some embodiments, the expression level of prolifin in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of prolifin in the biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of prolifin in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of calmodulin-2 is increased in a biological sample treated with a therapeutically active agent (e.g., a caveolin-1 peptide or derivative thereof) compared to a control sample. In some embodiments, the expression level of calmodulin-2 in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of calmodulin-2 in the biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of calmodulin-2 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of calreticulin is increased in a biological sample treated with a therapeutically active agent (e.g., a caveolin-1 peptide or derivative thereof) compared to a control sample. In some embodiments, the expression level of calreticulin in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of calreticulin in the biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of calreticulin in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis (IPF). In some embodiments, the biological sample is fibroblasts. In some embodiments, the biological sample is IPF fibroblasts.

In some embodiments, the expression level of PPIA is increased in a biological sample treated with a therapeutically active agent (e.g., a caveolin-1 peptide or derivative thereof) compared to a control sample. In some embodiments, the expression level of PPIA in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of PPIA in the biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of PPIA in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of EIF5A1 is increased in a biological sample treated with a therapeutically active agent (e.g., a caveolin-1 peptide or derivative thereof) compared to a control sample. In some embodiments, the expression level of EIF5A1 in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of EIF5A1 in the biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of EIF5A1 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of LGALS7 is increased in a biological sample treated with a therapeutically active agent (e.g., a caveolin-1 peptide or derivative thereof) compared to a control sample. In some embodiments, the expression level of LGALS7 in the biological sample is increased by about 1.1-fold, about 1.2-fold, about 1.3-fold, about 1.4-fold, about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 500-fold, about 1,000-fold, or more compared to a control sample. In some embodiments, the expression level of LGALS7 in the biological sample is increased by about 110%, about 125%, about 150%, about 175%, about 200%, about 225%, about 250%, about 275%, about 300%, about 325%, about 350%, about 375%, about 400%, about 425%, about 450%, about 475%, about 500%, about 600%, about 700%, about 800%, about 900%, about 1000%, or more compared to a control sample. In some embodiments, the increased expression level of LGALS7 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is obtained from an elderly subject with a disease or disorder, e.g., interstitial lung disease.

In some embodiments, the expression level of a biomarker is decreased in a biological sample treated with a therapeutically active agent (e.g., a caveolin-1 peptide or derivative thereof) compared to a control sample. In some embodiments, the expression level of the biomarker in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of the biomarker in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis. In some embodiments, the biomarker is selected from the group consisting of MYDGF, soluble RAGE, phosphorylated SMAD2 (pSMAD2), pSMAD3, PDGFRβ, pPDGFRβ, PROF1, CALM2, CALR, PPIA, EIF5A, LGALS1, ANXA5, FABP5, GSTP1, ENO1, YWHAG, LGALS7, IL-11, MMP-2, CXCL7, sCD163, and p-mTOR.

In some embodiments, the expression level of pSMAD2 and/or pSMAD3 is decreased in a biological sample treated with a therapeutically active agent (e.g., a caveolin-1 peptide or derivative thereof) compared to a control sample. In some embodiments, the expression level of pSMAD2 and/or pSMAD3 in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of pSMAD2 and/or pSMAD3 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of PDGFRβ is decreased in a biological sample treated with a therapeutically active agent (e.g., a caveolin-1 peptide or derivative thereof) compared to a control sample. In some embodiments, the expression level of PDGFRβ in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of PDGFRβ in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of pPDGFRβ is decreased in a biological sample treated with a therapeutically active agent (e.g., a caveolin-1 peptide or derivative thereof) compared to a control sample. In some embodiments, the expression level of pPDGFRβ in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of pPDGFRβ in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of calreticulin is decreased in a biological sample treated with a therapeutically active agent (e.g., a caveolin-1 peptide or derivative thereof) compared to a control sample. In some embodiments, the expression level of calreticulin in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the increased expression level of calreticulin in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis (IPF). In some embodiments, the biological sample is epithelial cells. In some embodiments, the biological sample is IPF epithelial cells.

In some embodiments, the expression level of p-mTOR is decreased in a biological sample treated with a therapeutically active agent (e.g., a caveolin-1 peptide or derivative thereof) compared to a control sample. In some embodiments, the expression level of p-mTOR in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of p-mTOR in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis.

In some embodiments, the expression level of LGALS7 is decreased in a biological sample treated with a therapeutically active agent (e.g., a caveolin-1 peptide or derivative thereof) compared to a control sample. In some embodiments, the expression level of LGALS7 in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of LGALS7 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is obtained from an elderly subject with a disease or disorder, e.g., interstitial lung disease.

In some embodiments, the expression level of IL-11 is decreased in a biological sample treated with a therapeutically active agent (e.g., a caveolin-1 peptide or derivative thereof) compared to a control sample. In some embodiments, the expression level of IL-11 in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of IL-11 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is obtained from an elderly subject with a disease or disorder, e.g., interstitial lung disease.

In some embodiments, the expression level of MMP-2 is decreased in a biological sample treated with a therapeutically active agent (e.g., a caveolin-1 peptide or derivative thereof) compared to a control sample. In some embodiments, the expression level of MMP-2 in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of MMP-2 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is obtained from an elderly subject with a disease or disorder, e.g., interstitial lung disease.

In some embodiments, the expression level of CXCL7 is decreased in a biological sample treated with a therapeutically active agent (e.g., a caveolin-1 peptide or derivative thereof) compared to a control sample. In some embodiments, the expression level of CXCL7 in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of CXCL7 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is obtained from an elderly subject with a disease or disorder, e.g., interstitial lung disease.

In some embodiments, the expression level of SCD163 is decreased in a biological sample treated with a therapeutically active agent (e.g., a caveolin-1 peptide or derivative thereof) compared to a control sample. In some embodiments, the expression level of SCD163 in the biological sample is decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decreased expression level of SCD163 in the biological sample indicates a favorable response to the therapeutically active agent (e.g., a caveolin-1 peptide or derivative thereof). In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is obtained from an elderly subject with a disease or disorder, e.g., interstitial lung disease.

In some embodiments, the therapeutically active agent (e.g., a caveolin-1 peptide or derivative thereof) decreases receptor tyrosine kinase (RTK)-associated signaling pathways in a biological sample compared to a control sample. In some embodiments, the RTK-associated signaling pathways are decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decrease in RTK-associated signaling pathways is a decrease in the expression level of ALK, c-Jun, Herb2/ErbB3, Stat5a, PI3Kp110a, SRC-1, and/or YAP. In some embodiments, the decrease in RTK-associated signaling pathways is a decrease in the phosphorylation of ALK (p-ALK), c-Myc (p-c-Myc), EGFR (p-EGFR), MEK1/2 (p-MEK-1/2), MAPK (p-MAPK), PDK1 (p-PDK1), PDGFRb (p-PDGFRβ), RafB (p-RafB), Ret (p-Ret), Stat5a (p-Stat5a), and/or SRC (p-SRC). In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is fibroblasts obtained from a subject with a fibrotic disease (e.g., idiopathic pulmonary fibrosis).

In some embodiments, the therapeutically active agent (e.g., a caveolin-1 peptide or derivative thereof) decreases metabolic signaling pathways in a biological sample compared to a control sample. In some embodiments, the metabolic signaling pathways are decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decrease in metabolic signaling pathways is a decrease in the expression level of AMPKa, Deptor, LDHA, PFKFB3, and/or Raptor. In some embodiments, the decrease in metabolic signaling pathways is a decrease in the phosphorylation of AMPKa1 (p-AMPKa1), AMPKb1 (p-AMPKb1), mTOR (p-mTOR), Raptor (p-Raptor), and/or tuberin (p-tuberin). In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is fibroblasts obtained from a subject with a fibrotic disease (e.g., idiopathic pulmonary fibrosis).

In some embodiments, the therapeutically active agent (e.g., a caveolin-1 peptide or derivative thereof) decreases invasion-associated molecules in a biological sample compared to a control sample. In some embodiments, the invasion-associated molecules are decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decrease in invasion-associated molecules is a decrease in the expression level of TWIST2 and/or Wnt5ab.

In some embodiments, the therapeutically active agent (e.g., a caveolin-1 peptide or derivative thereof) decreases histone deacetylases (HDACs) in a biological sample compared to a control sample. In some embodiments, the HDACs are decreased by about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or more compared to a control sample. In some embodiments, the decrease in HDACs is a decrease in the expression level of HDAC4 and/or HDAC6. In some embodiments, the biological sample is obtained from a subject with a disease or disorder, e.g., interstitial lung disease. In some embodiments, the biological sample is obtained from a subject with idiopathic pulmonary fibrosis. In some embodiments, the biological sample is fibroblasts obtained from a subject with a fibrotic disease (e.g., idiopathic pulmonary fibrosis).

In some embodiments, the methods provided herein decrease RTK-associated signaling in a subject with a fibrotic disease. In some embodiments, the method comprises administering a caveolin-1 peptide or derivative thereof to a subject with a fibrotic disease. In some embodiments, the caveolin-1 peptide or derivative thereof comprises the amino acid sequence of any one of SEQ ID NOs: 1-110. In some embodiments, the caveolin-1 peptide or derivative thereof comprises the amino acid sequence of SEQ ID NO: 3. In some embodiments, the caveolin-1 peptide or derivative thereof comprises the amino acid sequence of SEQ ID NO: 4.

In some embodiments, the methods provided herein decrease RTK-associated signaling in fibrotic fibroblasts. In some embodiments, the method comprises administering a caveolin-1 peptide or derivative thereof to the fibrotic fibroblasts. In some embodiments, the caveolin-1 peptide or derivative thereof does not affect RTK-associated signaling in normal fibroblasts. In some embodiments, the caveolin-1 peptide or derivative thereof decreases RTK-associated signaling in a normal fibroblast to a lesser extent than in fibrotic fibroblasts. In some embodiments, the caveolin-1 peptide or derivative thereof comprises the amino acid sequence of any one of SEQ ID NOs: 1-110. In some embodiments, the caveolin-1 peptide or derivative thereof comprises the amino acid sequence of SEQ ID NO: 3. In some embodiments, the caveolin-1 peptide or derivative thereof comprises the amino acid sequence of SEQ ID NO: 4.

In some embodiments, the methods provided herein decrease metabolic signaling in a subject with a fibrotic disease. In some embodiments, the method comprises administering a caveolin-1 peptide or derivative thereof to a subject with a fibrotic disease. In some embodiments, the caveolin-1 peptide or derivative thereof comprises the amino acid sequence of any one of SEQ ID NOs: 1-110. In some embodiments, the caveolin-1 peptide or derivative thereof comprises the amino acid sequence of SEQ ID NO: 3. In some embodiments, the caveolin-1 peptide or derivative thereof comprises the amino acid sequence of SEQ ID NO: 4.

In some embodiments, the methods provided herein decrease metabolic signaling in fibrotic fibroblasts. In some embodiments, the method comprises administering a caveolin-1 peptide or derivative thereof to the fibrotic fibroblasts. In some embodiments, the caveolin-1 peptide or derivative thereof does not affect metabolic signaling in normal fibroblasts. In some embodiments, the caveolin-1 peptide or derivative thereof decreases metabolic signaling in a normal fibroblast to a lesser extent than in fibrotic fibroblasts. In some embodiments, the caveolin-1 peptide or derivative thereof comprises the amino acid sequence of any one of SEQ ID NOs: 1-110. In some embodiments, the caveolin-1 peptide or derivative thereof comprises the amino acid sequence of SEQ ID NO: 3. In some embodiments, the caveolin-1 peptide or derivative thereof comprises the amino acid sequence of SEQ ID NO: 4.

In some embodiments, one or more biomarkers are identified to be associated with a therapeutically active agent in a subject with a disease or disorder, e.g., interstitial lung disease. In some embodiments, one or more biomarkers are identified to be associated caveolin-1 therapy in a subject with idiopathic pulmonary fibrosis.

In some embodiments, one or more biomarkers are used to determine or predict optimal dosing of a therapeutically active agent in a subject with a disease or disorder, e.g., interstitial lung disease. In some embodiments, one or more biomarkers are used to determine or predict optimal dosing of a caveolin-1 peptide or a derivative thereof (e.g., CSP-7 or Var55) in a subject with idiopathic pulmonary fibrosis.

### Subjects

The term “subject” as used herein refers to a mammalian subject. In some embodiments, the subject is a human, a companion animal, non-domestic livestock, or a zoo animal. In some embodiments, the subject is a human, mouse, rat, rabbit, hamster, guinea pig, dog, cat, cow, pig, sheep, goat, horse, or monkey. In some embodiments, the subject is human.

In some embodiments, the subject has a disease or disorder. In some embodiments, the subject with the disease or disorder is being treated with a therapeutically active agent, e.g., caveolin-1 peptide or a derivative thereof. In some embodiments, the biomarkers described herein are associated with the therapeutically active agent, e.g., caveolin-1 peptide or a derivative thereof, in a subject with a disease or disorder. Examples of diseases or disorders include, but are not limited to, autoimmune disorders (e.g., diabetes, lupus, multiple sclerosis, psoriasis, rheumatoid arthritis); inflammatory disorders (e.g., arthritis, pelvic inflammatory disease); infectious diseases (e.g., viral infections (e.g., HIV, HCV, RSV, COVID-19)), bacterial infections, fungal infections, sepsis); neurological disorders (e.g., Alzheimer's disease, Huntington's disease; autism; Duchenne muscular dystrophy); cardiovascular disorders (e.g., atherosclerosis, hypercholesterolemia, thrombosis, clotting disorders, angiogenic disorders such as macular degeneration); proliferative disorders (e.g., cancer, benign neoplasms); respiratory disorders (e.g., chronic obstructive pulmonary disease); digestive disorders (e.g., inflammatory bowel disease, ulcers); musculoskeletal disorders (e.g., fibromyalgia, arthritis); endocrine, metabolic, and nutritional disorders (e.g., diabetes, osteoporosis); urological disorders (e.g., renal disease); psychological disorders (e.g., depression, schizophrenia); skin disorders (e.g., wounds, eczema); blood and lymphatic disorders (e.g., anemia, hemophilia); fibrotic disorders (e.g., idiopathic pulmonary fibrosis); or ocular disorders (e.g., glaucoma, diabetic retinopathy).

In some embodiments, the subject has a fibrotic disease. In some embodiments, the subject with fibrotic disease is being treated with a therapeutically active agent, e.g., caveolin-1 peptide or a derivative thereof. In some embodiments, the biomarkers described herein are associated with the therapeutically active agent, e.g., caveolin-1 peptide or a derivative thereof, in a subject with fibrotic disease. Fibrotic diseases include those of the fibrotic diseases include those of the bone marrow, gallbladder, blood vessels, heart, joints, kidney, liver, lung, muscle, pancreas, penis, skin, soft tissue, eye, adrenal glands, thyroids and/or uterus. Exemplary of fibrotic diseases, include but are not limited to, pulmonary fibrosis, acute interstitial pneumonitis, nonspecific interstitial pneumonia, lymphocytic interstitial pneumonia, asthma, idiopathic interstitial fibrosis, interstitial lung disease, interstitial pneumonitis, cystic fibrosis, cryptogenic organizing pneumonia, desquamative interstitial pneumonia, diffuse parenchymal lung disease, respiratory bronchiolitis, subepithelial fibrosis, systemic sclerosis, cardiac fibrosis, myocardial fibrosis, endomyocardial fibrosis, pancreatic fibrosis, chronic pancreatitis, hepatic cirrhosis, chronic kidney disease, cirrhosis of the gallbladder, renal sclerosis, glomerulonephritis, arteriosclerosis, atherosclerosis, restenosis, macular degeneration, ocular scarring, cataracts, retinal and vitreal retinopathy, Grave's ophthalmopathy, neurofibromatosis, scleroderma, glioblastoma, keloid scarring, hypertrophic scarring, colloid and hypertrophic scarring, scarring after surgery, aberrant wound healing, peritoneal fibrosis, retroperitoneal fibrosis, chronic obstructive pulmonary disease, post-operative fibroids, diabetic nephropathy, nephrogenic systemic fibrosis, gynecological cancer, chronic myeloproliferative disorders, myelofibrosis, myeloproliferative syndrome, myeloid leukemia, myelodysplastic syndrome, inflammatory bowel disease (e.g, Crohn's disease and collagenous colitis), alcoholic steatohepatitis, alcoholic fatty liver disease, non-alcoholic fatty liver disease, fibrosarcoma, rheumatoid arthritis, rheumatoid pannus formation in rheumatoid arthritis or osteoarthritis, fibrosis as a result of Graft-Versus-Host Disease, organ transplant fibrosis, anthrofibrosis, non-alcoholic steatohepatitis, Alport syndrome, chronic COVID syndrome, glial scar formation in HIV, Peyronie's disease, associated cognitive motor disease and spongiform encephalopathy, gingival hypertrophy secondary to drugs and fibrocystic disease, Dupuytren's contracture, morphea, endometriosis, uterine fibroids, fibromyalgia, multifocal fibrosclerosis, fibrodysplasia ossificans progressiva, osteoporosis, or osteosclerosis.

In some embodiments, the subject has a pulmonary disease. In some embodiments, the subject with the pulmonary disease is being treated with a therapeutically active agent, e.g., caveolin-1 peptide or a derivative thereof. In some embodiments, the biomarkers described herein are associated with the therapeutically active agent, e.g., caveolin-1 peptide or a derivative thereof, in a subject with the pulmonary disease. Examples of pulmonary diseases, include but are not limited to, pulmonary fibrosis, interstitial lung disease, chronic obstructive pulmonary disease (COPD), acute lung injury, acute respiratory distress syndrome, chronic bronchitis, emphysema, lung cancer, cystic fibrosis, bronchiectasis, pneumonia, pleural effusion, tuberculosis, pulmonary hypertension, collagen vascular lung disease (e.g., from lupus, scleroderma, or mixed connective tissue disease), and asthma. Pulmonary diseases may be caused by smoking, exposure to irritants (e.g., asbestos and silica), allergy, genetics, or unknown causes.

In some embodiments, the subject has interstitial lung disease (ILD). In some embodiments, the subject with ILD is being treated with a therapeutically active agent, e.g., caveolin-1 peptide or a derivative thereof. In some embodiments, the biomarkers described herein are associated with the therapeutically active agent, e.g., caveolin-1 peptide or a derivative thereof, in a subject with ILD. Examples of ILDs include, but are not limited to, idiopathic pulmonary fibrosis, lymphangioleiomyomatosis, nonspecific interstitial pneumonia, cryptogenic organizing pneumonia; acute interstitial pneumonia, respiratory bronchiolitis-associated interstitial lung disease, desquamative interstitial pneumonia, lymphocytic interstitial pneumonia, and pulmonary sarcoidosis. ILDs can be associated with a connective tissue disease selected from systemic sclerosis, polymyositis, systemic lupus erythematosus, or rheumatoid arthritis. ILDs can also be drug-induced, e.g., induced by antibiotics, chemotherapeutic agents, antiarrhythmia agents, statins, and the like. ILDs can also result from a viral infection (e.g., COVID-19), a bacterial infection, or tuberculosis. ILDs can be associated with environmental or occupational exposure to silica, asbestos, talc, hard metals, inorganic or organic dust, radiation, gas/fumes, and the like.

In some embodiments, the subject has pulmonary fibrosis. In some embodiments, the subject with pulmonary fibrosis is being treated with a therapeutically active agent, e.g., caveolin-1 peptide or a derivative thereof. In some embodiments, the biomarkers described herein are associated with the therapeutically active agent, e.g., caveolin-1 peptide or a derivative thereof, in a subject with pulmonary fibrosis. Examples of pulmonary fibrosis, include but are not limited to, familial pulmonary fibrosis, interstitial lung disease, idiopathic pulmonary fibrosis, idiopathic nonspecific interstitial pneumonia, conventional interstitial pneumonia, cryptogenic organizing pneumonia, pulmonary sarcoidosis, fibrosing alveolitis, cystic fibrosis, COPD, adult respiratory distress syndrome, emphysema. Pulmonary fibrosis may be caused by many conditions, including chronic inflammatory processes (e.g., sarcoidosis and Wegener's granulomatosis), infections (e.g., COVID-19), environmental agents (e.g., asbestos, silica, hard metals, talc, and exposure to certain gases), exposure to ionizing radiation (e.g., radiation therapy to treat tumors of the chest), chronic conditions (e.g., lupus, rheumatoid arthritis), and even certain medications.

In some embodiments, the subject has idiopathic pulmonary fibrosis. In some embodiments, the subject with idiopathic pulmonary fibrosis is being treated with a therapeutically active agent, e.g., caveolin-1 peptide or a derivative thereof. In some embodiments, the biomarkers described herein are associated with the therapeutically active agent, e.g., caveolin-1 peptide or a derivative thereof, in a subject with idiopathic pulmonary fibrosis.

### Biological Samples

As used herein, the term “biological sample” refers to a sample obtained from a subject. A biological sample may be obtained from a subject using methods known to those skilled in the art. Examples of biological samples include, but are not limited to, body fluid, organs, tissues, and cells isolated from mammals. In some embodiments, the biological sample is a section of a biological sample, e.g., sections of an organ or tissue. In some embodiments, the biological sample is an extract from a biological sample, e.g., an antigen from a biological fluid. Methods are well known in the art for collecting, handling, and processing biological samples obtained from a subject.

In some embodiments, the biological sample is obtained from a mammal (e.g., rat, mouse, rabbit, dog, cat, cow, horse, donkey, guinea pig, monkey, or human). In some embodiments, the biological sample is obtained from a primate (e.g., chimpanzee or human). In some embodiments, the biological sample is obtained from a human. In some embodiments, the biological sample is obtained from a human with a disease or disorder, e.g., interstitial lung disease. In some embodiments, the biological sample is obtained from a human with idiopathic pulmonary fibrosis. In some embodiments, the biological sample is obtained from a human with no evidence of a disease or disorder (e.g., a control sample from a healthy human subject).

In some embodiments, the biological sample is a bodily fluid. Examples of bodily fluid include, but are not limited to, blood, plasma, serum, urine, peritoneal fluid, cerebral spinal fluid, amniotic fluid, bronchoalveolar lavage fluid, lymph, saliva, pleural effusions, or interstitial fluid. In some embodiments, the biological sample is plasma. In some embodiments, the biological sample is serum. In some embodiments, the biological sample is bronchoalveolar lavage fluid.

In some embodiments, the biological sample is a tissue or cell sample. The source of the tissue or cell sample may be solid tissue as from a fresh, frozen and/or preserved organ or tissue sample or biopsy or aspirate; cells from any time in gestation or development of the subject. The biological sample may also be cultured primary cells or cell lines derived from a subject with a disease or disorder, e.g., interstitial lung disease.

In some embodiments, the biological sample is a tissue sample obtained from a subject. In some embodiments, the biological sample is lung tissue, skin tissue, heart tissue, liver tissue, gastrointestinal tissue, pancreatic tissue, bone tissue, nervous tissue, adipose tissue, cartilage tissue, connective tissue, muscle tissue, epithelial tissue, or ocular tissue. In some embodiments, the biological sample is lung tissue from a subject with idiopathic pulmonary fibrosis.

In some embodiments, the biological sample is a cell sample obtained from a subject. In some embodiments, the biological sample is fibroblasts, conditionally reprogrammed cells, type I alveolar epithelial cells, type II alveolar epithelial cells, basal cells, Clara cells, ciliated cells, goblet cells, neuroendocrine cells, endothelial cell, bialveolar stem cells (BASC), alveolar macrophages, ionocytes, pericytes, mesothelial cells, red blood cells, white blood cells, peripheral blood mononuclear cells, B-cells, plasma cells, innate lymphoid cells, T-cells (e.g., CD8+ T cells, CD4+ T cells, and T regulatory cells), macrophages, non-classical monocytes, monocytes, neutrophils, eosinophils, dendritic cells, mast cells, NK cells, stem cells, cholangiocytes, hepatocytes, Kupffer cells, keratinocytes, melanocytes, cone cells, rod cells, pigmented cells, Langerhan cells, cardiomyocytes, smooth muscle cells, myofibroblasts, stromal cells, bone cells, adipocytes, muscle cells, epithelial cells, endocrine cells, germ cells, vascular cells, neuronal cells, glial cells, trophoblast cells, mesenchymal cells, cancer cells (including cancer cell lines), skin cells, nerve cells, or pancreatic cells. In some embodiments, the biological sample is a fibroblast from a subject with idiopathic pulmonary fibrosis. In some embodiments, the biological sample is a basal-like cell from a subject with idiopathic pulmonary fibrosis. In some embodiments, the biological sample is a conditionally reprogrammed cell from a subject with idiopathic pulmonary fibrosis. In some embodiments, the biological sample is an epithelial cell from a subject with idiopathic pulmonary fibrosis.

In some embodiments, the biological sample is supernatant from cells or tissue cultured in vitro or ex vivo. In some embodiments, the supernatant is from cultured conditionally reprogrammed cells obtained from a subject with idiopathic pulmonary fibrosis. In some embodiments, the supernatant is from cultured lung tissue from a subject with idiopathic pulmonary fibrosis. In some embodiments, the supernatant is from cultured fibroblasts from a subject with idiopathic pulmonary fibrosis.

In some embodiments, the biological sample is a control sample. The term “control sample” as used herein refers to a biological sample obtained from a subject or population of subjects not treated with the therapeutically active agents described herein (e.g., a caveolin-1 peptide or derivative thereof). In some embodiments, the control sample is used in determining or identifying the expression level of a biomarker in a biological sample obtained from a subject treated with the therapeutically active agents described herein (e.g., a caveolin-1 peptide or derivative thereof). In some embodiments, the control sample is a biological sample obtained from a subject or a population of subjects with a disease or disorder, e.g., interstitial lung disease. In some embodiments, the control sample is a biological sample obtained from a healthy subject. In some embodiments, the control sample is a biological sample obtained from a subject prior to treatment with a therapeutically active agent. In some embodiments, the control sample is a biological sample obtained from a subject prior to treatment with a caveolin-1 peptide or derivative thereof. In some embodiments, the control sample is a biological sample obtained from a subject prior to treatment with CSP-7 or Var55.

In some embodiments, the biological sample contains compounds which are not naturally intermixed with the biological sample. Examples of such compounds include, but are not limited to, preservatives, anticoagulants, buffers, fixatives, nutrients, antibiotics, or the like.

### Uses of Biomarkers for Caveolin-1 Peptide Therapy

In some embodiments, the present disclosure provides a method for treating a subject with caveolin-1 peptide therapy, wherein the subject is suffering from fibrosis, the method comprising the steps of: (a) obtaining or having obtained a biological sample from the subject; (b) treating or having treated a cell of the biological sample with a caveolin-1 peptide or derivative thereof; (c) measuring an expression level of a biomarker in the cell; and (d) comparing the expression level of the biomarker to a control sample; wherein the biomarker is myeloid-derived growth factor (MYDGF), soluble RAGE, phosphorylated mothers against decapentaplegic homolog 2/3 (pSMAD2/3), platelet-derived growth factor receptor beta (PDGFRβ), galectin-7 (LGALS7), interleukin-11 (IL-11), matrix metalloproteinase-2 (MMP-2), chemokine ligand 7 (CXCL-7), soluble CD163, phosphorylated mTOR (p-mTOR), phosphorylated PDGFRβ (pPDGFRβ), prolifin (PROF1), calmodulin 2 (CALM2), calreticulin (CALR), peptidyl-prolyl cis-trans isomerase A (PPIA), or eukaryotic translation initiation factor 5A (EIF5A1); and
if the expression level of MYDGF, PROF1, CALM2, CALR, PPIA, IF5A1, LGALS7, soluble RAGE, or EIF5A1 is increased, then administering caveolin-1 peptide or derivative thereof to the subject; or
if the expression level of PDGFRβ, pPDGFRβ, p-mTOR, CALR, IL-11, MMP-2, CXCL7, soluble CD163, or pSMAD2/3 is decreased, then administering caveolin-1 peptide or derivative thereof to the subject.

In some embodiments, the method of administering caveolin-1 peptide or derivative thereof is as described herein (e.g., identity of Cav-1 peptides, dosing, administration route, subject, etc).

In some embodiments, the biological samples, types of cells, measurement of biomarkers, and/or levels of biomarker expression changes are as described herein.

In some embodiment, steps (b)-(d) of the method are repeated one or more times.

In some embodiments, wherein fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome. In some embodiments, interstitial lung disease is idiopathic pulmonary fibrosis, lymphangioleiomyomatosis, nonspecific interstitial pneumonia, idiopathic interstitial pneumonia, cryptogenic organizing pneumonia, acute interstitial pneumonia, respiratory bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease. In some embodiments, the interstitial lung disease is idiopathic pulmonary fibrosis.

The method of claim 1, wherein the control sample is a cell obtained from the subject prior to treatment with the caveolin-1 peptide or derivative thereof.

In some embodiments, the methods provided herein are used to identify an altered expression level of a biomarker associated with a therapeutically active agent in a subject with a disease or disorder. In some embodiments, the method comprises: (a) treating a biological sample from the subject with the therapeutically active agent; (b) measuring an expression level of a biomarker in the biological sample; and (c) comparing the expression level of the biomarker in the biological sample to the expression level of the biomarker in a control sample. In some embodiments, the therapeutically active agent modulates the expression level of the biomarker in the biological sample. In some embodiments, the biomarker is selected from the group consisting of: MYDGF, soluble RAGE, pSMAD2/3, PDGFRβ, pPDGFRβ, PROF1, CALM2, CALR, PPIA, IF5A1, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the increased expression level of MYDGF, PROF1, CALM2, CALR, PPIA, EIF5A1, LGALS7, and/or soluble RAGE is associated with the therapeutically active agent in a subject with a disease or disorder. In some embodiments, the decreased expression level of PDGFRβ, CALR, pPDGFRβ, p-mTOR, IL-11, MMP-2, CXCL7, sCD163, or pSMAD2/3 is associated with the therapeutically active agent in a subject with a disease or disorder.

In some embodiments, the methods provided herein are used to identify an altered expression level of a biomarker associated with caveolin-1 therapy in a subject with interstitial lung disease. In some embodiments, the method comprises: (a) treating a cell from the subject with interstitial lung disease with a caveolin-1 peptide or derivative thereof; (b) measuring an expression level of a biomarker in the cell; and (c) comparing the expression level of the biomarker in the cell to the expression level of the biomarker in a control sample. In some embodiments, the caveolin-1 peptide or derivative thereof modulates the expression level of the biomarker in the cell. In some embodiments, the biomarker is selected from the group consisting of: MYDGF, soluble RAGE, pSMAD2/3, PDGFRβ, pPDGFRβ, PROF1, CALM2, CALR, PPIA, IF5A1, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the increased expression level of MYDGF, PROF1, CALM2, CALR, PPIA, EIF5A1, LGALS7, and/or soluble RAGE is associated with the caveolin-1 peptide or derivative thereof in a subject with a disease or disorder. In some embodiments, the decreased expression level of PDGFRβ, CALR, pPDGFRβ, p-mTOR, IL-11, MMP-2, CXCL7, sCD163, or pSMAD2/3 is associated with the caveolin-1 peptide or derivative thereof in a subject with a disease or disorder. In some embodiments, the interstitial lung disease is idiopathic pulmonary fibrosis.

In some embodiments, the methods provided herein are used to identify an altered expression level of a biomarker associated with CSP-7 treatment in a subject with interstitial lung disease. In some embodiments, the method comprises: (a) treating a cell from the subject with interstitial lung disease with CSP-7; (b) measuring an expression level of a biomarker in the cell; and (c) comparing the expression level of the biomarker in the cell to the expression level of the biomarker in a control sample. In some embodiments, CSP-7 modulates the expression level of the biomarker in the cell. In some embodiments, the biomarker is selected from the group consisting of: MYDGF, soluble RAGE, pSMAD2/3, PDGFRβ, pPDGFRβ, PROF1, CALM2, CALR, PPIA, IF5A1, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the increased expression level of MYDGF, PROF1, CALM2, CALR, PPIA, EIF5A1, LGALS7, and/or soluble RAGE is associated with CSP-7 in a subject interstitial lung disease. In some embodiments, the decreased expression level of PDGFRβ, CALR, pPDGFRβ, p-mTOR, IL-11, MMP-2, CXCL7, sCD163, or pSMAD2/3 is associated with CSP-7 in a subject with interstitial lung disease. In some embodiments, the interstitial lung disease is idiopathic pulmonary fibrosis.

In some embodiments, the methods provided herein are used to identify an altered expression level of a biomarker associated with Var55 treatment in a subject with interstitial lung disease. In some embodiments, the method comprises: (a) treating a cell from the subject with interstitial lung disease with Var55; (b) measuring an expression level of a biomarker in the cell; and (c) comparing the expression level of the biomarker in the cell to the expression level of the biomarker in a control sample. In some embodiments, Var55 modulates the expression level of the biomarker in the cell. In some embodiments, the biomarker is selected from the group consisting of: MYDGF, soluble RAGE, pSMAD2/3, PDGFRβ, pPDGFRβ, PROF1, CALM2, CALR, PPIA, IF5A1, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the increased expression level of MYDGF, PROF1, CALM2, CALR, PPIA, EIF5A1, LGALS7, and/or soluble RAGE is associated with Var55 in a subject with interstitial lung disease. In some embodiments, the decreased expression level of PDGFRβ, CALR, pPDGFRβ, p-mTOR, IL-11, MMP-2, CXCL7, sCD163, or pSMAD2/3 is associated with Var55 in a subject with interstitial lung disease. In some embodiments, the interstitial lung disease is idiopathic pulmonary fibrosis.

In some embodiments, the methods provided herein are used to predict or determine efficacy of a therapeutically active agent in a subject with a disease or disorder. In some embodiments, the method comprises: (a) treating a biological sample from the subject with the therapeutically active agent; (b) measuring an expression level of a biomarker in the biological sample; and (c) comparing the expression level of the biomarker in the biological sample to the expression level of the biomarker in a control sample. In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the increased expression level of MYDGF, PROF1, CALM2, CALR, PPIA, EIF5A1, LGALS7, or soluble RAGE indicates a favorable response to the therapeutically active agent. In some embodiments, the decreased expression level of PDGFRβ, CALR, pPDGFRβ, p-mTOR, IL-11, MMP-2, CXCL7, sCD163, or pSMAD2/3 indicates a favorable response to the therapeutically active agent.

In some embodiments, the methods provided herein are used to predict or determine efficacy of a caveolin-1 peptide or derivative thereof in a subject with interstitial lung disease. In some embodiments, the method comprises: (a) treating a biological sample from the subject with interstitial lung disease with the caveolin-1 peptide or derivative thereof; (b) measuring an expression level of a biomarker in the biological sample; and (c) comparing the expression level of the biomarker in the biological sample to the expression level of the biomarker in a control sample. In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the increased expression level of MYDGF, PROF1, CALM2, CALR, PPIA, EIF5A1, LGALS7, or soluble RAGE indicates a favorable response to the caveolin-1 peptide or derivative thereof. In some embodiments, the decreased expression level of PDGFRβ, CALR, pPDGFRβ, p-mTOR, IL-11, MMP-2, CXCL7, sCD163, or pSMAD2/3 indicates a favorable response to the caveolin-1 peptide or derivative thereof. In some embodiments, the biological sample is a lung fibroblast or basal-like cell obtained from a subject with interstitial lung disease. In some embodiments, the interstitial lung disease is idiopathic pulmonary fibrosis.

In some embodiments, the methods provided herein are used to predict or determine efficacy of CSP-7 in a subject with interstitial lung disease. In some embodiments, the method comprises: (a) treating a biological sample from the subject with CSP-7; (b) measuring an expression level of a biomarker in the biological sample; and (c) comparing the expression level of the biomarker in the biological sample to the expression level of the biomarker in a control sample. In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the increased expression level of MYDGF, PROF1, CALM2, CALR, PPIA, EIF5A1, LGALS7, or soluble RAGE indicates a favorable response to CSP-7. In some embodiments, the decreased expression level of PDGFRβ, pPDGFRβ, p-mTOR, IL-11, MMP-2, CXCL7, sCD163, pSMAD2/3, or CALR indicates a favorable response to CSP-7. In some embodiments, the biological sample is a lung fibroblast or basal-like cell obtained from a subject with interstitial lung disease. In some embodiments, the interstitial lung disease is idiopathic pulmonary fibrosis.

In some embodiments, the methods provided herein are used to predict or determine efficacy of Var55 in a subject with interstitial lung disease. In some embodiments, the method comprises: (a) treating a biological sample from the subject with Var55; (b) measuring an expression level of a biomarker in the biological sample; and (c) comparing the expression level of the biomarker in the biological sample to the expression level of the biomarker in a control sample. In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the increased expression level of MYDGF, PROF1, CALM2, CALR, PPIA, EIF5A1, LGALS7, or soluble RAGE indicates a favorable response to Var55. In some embodiments, the decreased expression level of PDGFRβ, CALR, pPDGFRβ, p-mTOR, IL-11, MMP-2, CXCL7, sCD163, or pSMAD2/3 indicates a favorable response to Var55. In some embodiments, the biological sample is a lung fibroblast or basal-like cell obtained from a subject with interstitial lung disease. In some embodiments, the interstitial lung disease is idiopathic pulmonary fibrosis.

In some embodiments, the methods provided herein are used to determine an optimal dose of a therapeutically active agent in a subject with a disease or disorder. In some embodiments, the method of determining an optimal dose of a therapeutically active agent in a subject with a disease or disorder comprises: (a) obtaining a biological sample from a subject prior to treatment with the therapeutically active agent; (b) measuring an expression level of a biomarker in the biological sample from the subject; (c) comparing the expression level of the biomarker in the biological sample to the expression level of the biomarker in a control sample; and (d) determining an optimal dose of the therapeutically active agent to be administered to the subject based on the expression level of the biomarker in step (c). In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ.

In some embodiments, the methods provided herein are used to determine an optimal dose of a caveolin-1 peptide or derivative thereof in a subject with interstitial lung disease. In some embodiments, the method of determining an optimal dose of a caveolin-1 peptide or derivative thereof in a subject with interstitial lung disease comprises: (a) obtaining a biological sample from a subject prior to treatment with the caveolin-1 peptide or derivative thereof; (b) measuring an expression level of a biomarker in the biological sample from the subject; (c) comparing the expression level of the biomarker in the biological sample to the expression level of the biomarker in a control sample; and (d) determining an optimal dose of the caveolin-1 peptide or derivative thereof to be administered to the subject based on the expression level of the biomarker in step (c). In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the interstitial lung disease is idiopathic pulmonary fibrosis.

In some embodiments, the methods provided herein are used to determine an optimal dose of CSP-7 in a subject with interstitial lung disease. In some embodiments, the method of determining an optimal dose of CSP-7 in a subject with interstitial lung disease comprises: (a) obtaining a biological sample from a subject prior to treatment with CSP-7; (b) measuring an expression level of a biomarker in the biological sample from the subject; (c) comparing the expression level of the biomarker in the biological sample to the expression level of the biomarker in a control sample; and (d) determining an optimal dose of CSP-7 to be administered to the subject based on the expression level of the biomarker in step (c). In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the interstitial lung disease is idiopathic pulmonary fibrosis.

In some embodiments, the methods provided herein are used to determine an optimal dose of Var55 in a subject with interstitial lung disease. In some embodiments, the method of determining an optimal dose of Var55 in a subject with interstitial lung disease comprises: (a) obtaining a biological sample from a subject prior to treatment with Var55; (b) measuring an expression level of a biomarker in the biological sample from the subject; (c) comparing the expression level of the biomarker in the biological sample to the expression level of the biomarker in a control sample; and (d) determining an optimal dose of Var55 to be administered to the subject based on the expression level of the biomarker in step (c). In some embodiments, the biomarker is selected from the group consisting of: PDGFRβ, pPDGFRβ, pSMAD2/3, MYDGF, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the interstitial lung disease is idiopathic pulmonary fibrosis.

In some embodiments of any one of the methods as provided herein, the biomarker is selected from the group consisting of: PDGFRβ, pPDGFRβ, pSMAD2/3, MYDGF, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments of any one of the methods as provided herein, the biomarker is selected from the group consisting of: MYDGF, soluble RAGE, pSMAD2/3, PDGFRβ, PROF1, CALM2, CALR, PPIA, IF5A1, LGALS1, ANXA5, FABP5, GSTP1, ENO1, and YWHAG.

In some embodiments, one or more biological samples are collected from a subject with a disease or disorder following treatment with a therapeutically active agent. In some embodiments, an expression level of a biomarker in the biological sample is measured and compared to a control sample (e.g., a biological sample obtained from the subject prior to treatment with the therapeutically active agent). In some embodiments, the expression level of the biomarker is used to determine efficacy of the therapeutically active agent in the subject. In some embodiments, the expression level of the biomarker is used to determine optimal dosing of the therapeutically active agent in the subject. In some embodiments, the expression level of the biomarker is used to monitor dosing of the therapeutically active agent. In some embodiments, the therapeutically active agent is a caveolin-1 peptide or a derivative thereof, e.g., CSP-7 or Var55.

In some embodiments, a first biological sample and a second biological sample are obtained from a subject with a disease or disorder, e.g., interstitial lung disease. In some embodiments, the first biological sample is obtained from a subject prior to treatment with a therapeutically active agent. In some embodiments, the second biological sample is obtained from the subject following treatment with the therapeutically active agent. In some embodiments, one or more additional biological samples are obtained from the subject following treatment with the therapeutically active agent. In some embodiments, the second biological sample is obtained from the subject about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 8 months, or about 1 year following treatment with the therapeutically active agent. In some embodiments, the one or more additional biological samples are obtained from the subject about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 8 months, or about 1 year following treatment with the therapeutically active agent. In some embodiments, the therapeutically active agent is a caveolin-1 peptide or derivative thereof, e.g., CSP-7 or Var55.

The methods provided herein can be used to predict or determine the efficacy of a therapeutically active agent in a subject with a disease or disorder. In some embodiments, the method of predicting or determining the efficacy of a therapeutically active agent in a subject with a disease or disorder comprises: (a) obtaining a first biological sample from a subject prior to treatment with a therapeutically active agent; (b) administering the therapeutically active agent to the subject; (c) obtaining a second biological sample from the subject following treatment with the therapeutically active agent; (d) measuring an expression level of a biomarker in the first biological sample and the second biological sample; and (e) comparing the expression level of the biomarker in the first biological sample to the expression level of the biomarker in the second biological sample. In some embodiments, step (a) of the method further comprises determining the expression level of the biomarker in the first biological sample compared to a control sample prior to treatment with the therapeutically active agent. In some embodiments, the expression level of the biomarker in step (a) is used to determine an optimal dose of the therapeutically active agent administered in step (b). In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the increased expression level of MYDGF, PROF1, CALM2, CALR, PPIA, EIF5A1, LGALS7, or soluble RAGE indicates a favorable response to the therapeutically active agent. In some embodiments, the decreased expression level of PDGFRβ, CALR, pPDGFRβ, p-mTOR, IL-11, MMP-2, CXCL7, sCD163, or pSMAD2/3 indicates a favorable response to the therapeutically active agent.

In some embodiments, the methods provided herein are used to predict or determine the efficacy of a caveolin-1 peptide or derivative thereof in a subject with interstitial lung disease. In some embodiments, the method of predicting or determining the efficacy of a caveolin-1 peptide or derivative thereof in a subject with interstitial lung disease comprises: (a) obtaining a first biological sample from a subject prior to treatment with the caveolin-1 peptide or derivative thereof; (b) administering the caveolin-1 peptide or derivative thereof to the subject; (c) obtaining a second biological sample from the subject following treatment with the caveolin-1 peptide or derivative thereof; (d) measuring an expression level of a biomarker in the first biological sample and the second biological sample; and (e) comparing the expression level of the biomarker in the first biological sample to the expression level of the biomarker in the second biological sample. In some embodiments, step (a) of the method further comprises determining the expression level of the biomarker in the first biological sample compared to a control sample prior to treatment with the caveolin-1 peptide or derivative thereof. In some embodiments, the expression level of the biomarker in step (a) is used to determine an optimal dose of the caveolin-1 peptide or derivative thereof administered in step (b). In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the increased expression level of MYDGF, PROF1, CALM2, CALR, PPIA, EIF5A1, LGALS7, or soluble RAGE indicates a favorable response to the caveolin-1 peptide or derivative thereof. In some embodiments, the decreased expression level of PDGFRβ, pPDGFRβ, p-mTOR, IL-11, MMP-2, CXCL7, sCD163, or CALR, indicates a favorable response to the caveolin-1 peptide or derivative thereof. In some embodiments, the interstitial lung disease is idiopathic pulmonary fibrosis.

In some embodiments, the methods provided herein are used to predict or determine the efficacy of CSP-7 in a subject with interstitial lung disease. In some embodiments, the method of predicting or determining the efficacy of CSP-7 in a subject with interstitial lung disease comprises: (a) obtaining a first biological sample from a subject prior to treatment with CSP-7; (b) administering CSP-7 to the subject; (c) obtaining a second biological sample from the subject following treatment with CSP-7; (d) measuring an expression level of a biomarker in the first biological sample and the second biological sample; and (e) comparing the expression level of the biomarker in the first biological sample to the expression level of the biomarker in the second biological sample. In some embodiments, step (a) of the method further comprises determining the expression level of the biomarker in the first biological sample compared to a control sample prior to treatment with CSP-7. In some embodiments, the expression level of the biomarker in step (a) is used to determine an optimal dose of CSP-7 administered in step (b). In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is PDGFRβ. In some embodiments, the biomarker is pSMAD2/3. In some embodiments, the increased expression level of MYDGF, PROF1, CALM2, CALR, PPIA, EIF5A1, LGALS7, or soluble RAGE indicates a favorable response to CSP-7. In some embodiments, the decreased expression level of PDGFRβ, pPDGFRβ, p-mTOR, IL-11, MMP-2, CXCL7, sCD163, pSMAD2/3, or CALR indicates a favorable response to CSP-7. In some embodiments, the interstitial lung disease is idiopathic pulmonary fibrosis.

In some embodiments, the methods provided herein are used to predict or determine the efficacy of Var55 in a subject with interstitial lung disease. In some embodiments, the method of predicting or determining the efficacy of Var55 in a subject with interstitial lung disease comprises: (a) obtaining a first biological sample from a subject prior to treatment with Var55; (b) administering Var55 to the subject; (c) obtaining a second biological sample from the subject following treatment with Var55; (d) measuring an expression level of a biomarker in the first biological sample and the second biological sample; and (e) comparing the expression level of the biomarker in the first biological sample to the expression level of the biomarker in the second biological sample. In some embodiments, step (a) of the method further comprises determining the expression level of the biomarker in the first biological sample compared to a control sample prior to treatment with Var55. In some embodiments, the expression level of the biomarker in step (a) is used to determine an optimal dose of Var55 administered in step (b). In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the increased expression level of MYDGF, PROF1, CALM2, CALR, PPIA, EIF5A1, LGALS7, or soluble RAGE indicates a favorable response to Var55. In some embodiments, the decreased expression level of PDGFRβ, CALR, pPDGFRβ, p-mTOR, IL-11, MMP-2, CXCL7, sCD163, or pSMAD2/3 indicates a favorable response to Var55. In some embodiments, the interstitial lung disease is idiopathic pulmonary fibrosis.

In some embodiments, the methods provided herein are used to monitor treatment of a therapeutically active agent in a subject with a disease or disorder. In some embodiments, the method of monitoring treatment of a therapeutically active agent in a subject with a disease or disorder comprises: (a) obtaining a first biological sample from a subject prior to treatment with the therapeutically active agent; (b) measuring an expression level of a biomarker in the biological sample from the subject; (c) comparing the expression level of the biomarker in the biological sample to the expression level of the biomarker in a control sample; (d) administering the therapeutically active agent to the subject; (e) obtaining a second biological sample from the subject following treatment with the therapeutically active agent; (f) measuring the expression level of the biomarker in the second biological sample from the subject; (g) comparing the expression level of the biomarker in the first biological sample to the expression level of the biomarker in the second biological sample. In some embodiments, the dose of the therapeutically active agent will be increased if the expression level of the biomarker in the second biological sample is lower than the expression level of the biomarker in the first biological sample. In some embodiments, the dose of the therapeutically active agent will be increased if the expression level of the biomarker in the second biological sample is the same as the expression level of the biomarker in the first biological sample. In some embodiments, the dose of the therapeutically active agent will be maintained if the expression level of the biomarker in the second biological sample is higher than the expression level of the biomarker in the first biological sample. In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ.

In some embodiments, the methods provided herein are used to monitor treatment of a caveolin-1 peptide or derivative thereof in a subject with interstitial lung disease. In some embodiments, the method of monitoring treatment of a caveolin-1 peptide or derivative thereof in a subject with interstitial lung disease comprises: (a) obtaining a first biological sample from a subject prior to treatment with the caveolin-1 peptide or derivative thereof; (b) measuring an expression level of a biomarker in the biological sample from the subject; (c) comparing the expression level of the biomarker in the biological sample to the expression level of the biomarker in a control sample; (d) administering the caveolin-1 peptide or derivative thereof to the subject; (e) obtaining a second biological sample from the subject following treatment with the caveolin-1 peptide or derivative thereof; (f) measuring the expression level of the biomarker in the second biological sample from the subject; (g) comparing the expression level of the biomarker in the first biological sample to the expression level of the biomarker in the second biological sample. In some embodiments, the dose of the caveolin-1 peptide or derivative thereof will be increased if the expression level of the biomarker in the second biological sample is lower than the expression level of the biomarker in the first biological sample. In some embodiments, the dose of the caveolin-1 peptide or derivative thereof will be increased if the expression level of the biomarker in the second biological sample is the same as the expression level of the biomarker in the first biological sample. In some embodiments, the dose of the caveolin-1 peptide or derivative thereof will be maintained if the expression level of the biomarker in the second biological sample is higher than the expression level of the biomarker in the first biological sample. In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the interstitial lung disease is idiopathic pulmonary fibrosis.

In some embodiments, the methods provided herein are used to monitor treatment of CSP-7 in a subject with interstitial lung disease. In some embodiments, the method of monitoring treatment of CSP-7 in a subject with interstitial lung disease comprises: (a) obtaining a first biological sample from a subject prior to treatment with CSP-7; (b) measuring an expression level of a biomarker in the biological sample from the subject; (c) comparing the expression level of the biomarker in the biological sample to the expression level of the biomarker in a control sample; (d) administering CSP-7 to the subject; (e) obtaining a second biological sample from the subject following treatment with CSP-7; (f) measuring the expression level of the biomarker in the second biological sample from the subject; (g) comparing the expression level of the biomarker in the first biological sample to the expression level of the biomarker in the second biological sample. In some embodiments, the dose of CSP-7 will be increased if the expression level of the biomarker in the second biological sample is lower than the expression level of the biomarker in the first biological sample. In some embodiments, the dose of CSP-7 will be increased if the expression level of the biomarker in the second biological sample is the same as the expression level of the biomarker in the first biological sample. In some embodiments, the dose of CSP-7 will be maintained if the expression level of the biomarker in the second biological sample is higher than the expression level of the biomarker in the first biological sample. In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the interstitial lung disease is idiopathic pulmonary fibrosis.

In some embodiments, the methods provided herein are used to monitor treatment of Var55 in a subject with interstitial lung disease. In some embodiments, the method of monitoring treatment of Var55 in a subject with interstitial lung disease comprises: (a) obtaining a first biological sample from a subject prior to treatment with Var55; (b) measuring an expression level of a biomarker in the biological sample from the subject; (c) comparing the expression level of the biomarker in the biological sample to the expression level of the biomarker in a control sample; (d) administering Var55 to the subject; (e) obtaining a second biological sample from the subject following treatment with Var55; (f) measuring the expression level of the biomarker in the second biological sample from the subject; (g) comparing the expression level of the biomarker in the first biological sample to the expression level of the biomarker in the second biological sample. In some embodiments, the dose of Var55 will be increased if the expression level of the biomarker in the second biological sample is lower than the expression level of the biomarker in the first biological sample. In some embodiments, the dose of Var55 will be increased if the expression level of the biomarker in the second biological sample is the same as the expression level of the biomarker in the first biological sample. In some embodiments, the dose of Var55 will be maintained if the expression level of the biomarker in the second biological sample is higher than the expression level of the biomarker in the first biological sample. In some embodiments, the biomarker is selected from the group consisting of: PDGFRβ, pPDGFRβ, MYDGF, pSMAD2/3, PROF1, CALM2, CALR, PPIA, IF5A1, soluble RAGE, LGALS1, ANXA5, FABP5, GSTP1, ENO1, YWHAG, LGALS7, IL-11, MMP-2, CXCL7, sCD163, and p-mTOR. In some embodiments, the biomarker is MYDGF. In some embodiments, the biomarker is soluble RAGE. In some embodiments, the biomarker is pSMAD2/3. In some embodiments, the biomarker is PDGFRβ. In some embodiments, the interstitial lung disease is idiopathic pulmonary fibrosis.

### Methods of Biomarker Detection

The general methods of determining biomarker expression in a biological sample are well-known in the art. Methods of biomarker detection generally include those that quantify the level of a biomarker in a biological sample (quantitative method) or the presence or absence of a biomarker in a biological sample (qualitative method). It is generally known to the skilled artisan which of the following methods are suitable for qualitative and/or for quantitative detection of a biomarker. Biological samples can be assayed for, e.g., protein biomarkers using mass spectrometry and immunoassays, such as Western blots, ELISAs, radioimmunoassays (RIA), fluorescence-based immunoassays, immunoprecipitation assays, flow cytometry, or immunohistochemistry; and mRNA or DNA biomarkers using Northern blot, dot-blot, polymerase chain reaction (PCR) analysis, array hybridization (e.g., in situ hybridization), RNase protection assay, or using DNA SNP chip microarrays. Further suitable methods to detect biomarkers include measuring a physical or chemical property specific for the biomarker such as its precise molecular mass or NMR spectrum. Examples of methods that measure a physical or chemical property of the biomarker include, but are not limited to, enzymatic assays, cytological assays, biosensors, optical devices coupled to immunoassays, biochips, analytical devices such as mass-spectrometers, NMR-analyzers, and chromatography devices.

In some embodiments, the biomarker may be amplified by any method generally known in the art. The term “amplification” refers to the production of a plurality of biomarker molecules from a target biomarker. For example, if the biomarker is a nucleic acid, amplifying the nucleic acid refers to the production of a plurality of nucleic acid molecules from a target nucleic acid wherein primers hybridize to specific sites on the target nucleic acid molecules in order to provide an initiation site for extension by, e.g., a polymerase. Amplification can be carried out by any method generally known in the art, including, but not limited to, PCR-based amplification methods, such as standard PCR, long PCR, hot-start PCR, qPCR, and RT-PCR; isothermal amplification methods, such as nucleic acid sequence-based amplification (NASBA) and the transcription-mediated amplification (TMA); and hybridization signal amplification methods, such as the branched DNA assay method.

In some embodiments, the biomarkers described herein are detected or measured in a biological sample using quantitative real-time PCR (qRT-PCR). qRT-PCR is an amplification technique that can be used to measure levels of mRNA expression. (see, e.g., Gibson et al., 1996, Genome Research 6:995-1001, Heid et al., 1996, Genome Research 6:986-994). qRT-PCR evaluates the level of PCR product accumulation during amplification. This technique permits quantitative evaluation of mRNA levels in multiple samples. For mRNA levels, mRNA is extracted from a biological sample and cDNA is prepared using standard techniques. Primers and fluorescent probes can be designed for the biomarker of interest and optimal concentrations of primers and probes can be determined by those of ordinary skill in the art. Standard curves can be generated using the cycle threshold (Ct) values determined in the qRT-PCR, which are related to the initial concentration of the nucleic acid of interest used in the assay. The Ct value is determined by the cycle number at which exponential amplification of the cDNA molecule begins and is used to determine expression of the nucleic acid of interest (i.e., the biomarker) in the biological sample.

In some embodiments, the RNA or protein samples are normalized for differences in the amount of RNA or protein assayed, variability in the quality of the RNA or protein samples used, and/or variability between assay runs. In some embodiments, the RNA or protein sample is normalized by measuring and incorporating the expression of a housekeeping gene (also referred to as an internal control). Examples of housekeeping genes include, but are not limited to, PPIA, ACTB, GAPDH, TFRC, and 18S. In some embodiments, normalization is based on the mean or median signal of all of the assayed genes or a large subset thereof (global normalization approach). For example, the expression level of a biomarker (e.g., MYDGF) and of a housekeeping gene can be measured from a biological sample from a subject being treated for idiopathic pulmonary fibrosis using caveolin-1 peptide therapy. The resulting detection data of the biomarker can be normalized against the detection data for the housekeeping gene. The mean value and standard deviation for the subject can be calculated. The expression level of the same biomarker from a control sample (e.g., an untreated subject with idiopathic pulmonary fibrosis) can be detected using the same methods, and the mean value and standard deviation for the control sample can be calculated. The relative expression level of the biomarker can be compared between the subject being treated for idiopathic pulmonary fibrosis and the untreated subject with idiopathic pulmonary fibrosis.

In some embodiments, an mRNA biomarker is detected or measured in a biological sample using a DNA array, a chip, or a microarray. Oligonucleotides corresponding to the cDNA of the biomarker are immobilized on a chip which is then hybridized with labeled nucleic acids derived from a sample obtained from a patient. Positive hybridization signal is obtained with the sample containing biomarkers transcripts. For example, to detect or measure an mRNA biomarker, mRNA is extracted from the biological sample, reverse transcribed, and fluorescent-labeled cDNA probes are generated. The microarrays capable of hybridizing to the biomarker's cDNA are then probed with the labeled cDNA probes, the slides scanned and fluorescence intensity measured. This intensity correlates with the hybridization intensity and expression levels. Quantitative microarray hybridization assays are well known in the art (see, e.g., in U.S. Pat. Nos. 6,004,755 and 6,492,122).

In some embodiments, the biomarkers described herein are detected or measured using mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, e.g., U.S. Pat. App. Nos: 20030199001; 20030134304; and 20030077616.

In some embodiments, the biomarkers described herein are detected or measured in a biological sample using two-dimensional gel electrophoresis (2DE). 2DE separates proteins in the biological sample based on molecular charge and weight. The protein is first separated into its charges with isoelectric focusing (IEF) and then is further separated according to its mass by SDS-PAGE. The separated protein on the gel with IEF is negatively charged by treatment with SDS, and the electrophoresis is performed by inserting the gel horizontally into the SDS-PAGE gel. Thus, the proteins that are focused on the pI are separated according to their molecular weights. Using the system known as “ISO-DALT,” both IEF and SDS-PAGE can be carried out simultaneously. See, e.g., Buyukkoroglu et al., Omics Technologies and Bioengineering, 2018, pages 317-351.

Mass spectrometry methods are well known in the art and have been used to quantify and/or identify protein biomarkers (See, e.g., Li et al., 2000, Tibtech 18:151-160; Rowley et al., 2000, Methods 20:383-397; and Kuster and Mann, 1998, Curr. Opin. Structural Biol. 8:393-400). For additional disclosure regarding mass spectrometers, see, e.g., Principles of Instrumental Analysis, 3rd edition, Skoog, Saunders College Publishing, Philadelphia, 1985; and Kirk-Othmer Encyclopedia of Chemical Technology, 4.sup.th ed. Vol. 15, John Wiley & Sons, New York 1995, pp. 1071-1094.

In some embodiments, a gas phase ion spectrophotometer is used to analyze the biological sample. In some embodiments, laser-desorption/ionization mass spectrometry is used to analyze the biological sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. Nos. 5,118,937; and 5,045,694. In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

In some embodiments, the biomarkers described herein are detected or measured in a biological sample using a protein-binding agent. In some embodiments, a protein-binding agent is a ligand that specifically binds to a biomarker protein. Examples of protein-binding agents include, but are not limited to, synthetic peptides, chemicals, small molecules, and antibodies or fragments thereof.

In some embodiments, the biomarkers described herein are detected or measured in a biological sample using an immunoassay. The term “immunoassay” refers to any method of detection that uses at least one specific antibody for the detection and/or quantification of an antigen (e.g., the biomarkers disclosed herein). Examples of immunoassays include, but are not limited to, Western blots, enzyme-linked immunosorbent assay (ELISA), flow cytometry, radioimmunoassay (RIA), a competitive immunoassay, a noncompetitive immunoassay, fluorescence polarization immunoassay (FPIA), and enzyme multiplied immunoassay technique (EMIT). A wide range of immunoassay techniques have been previously described, see, e.g., U.S. Pat. Nos. 4,016,043; 4,424,279; and 4,018,653.

In some embodiments, the biomarkers described herein are detected or measured in a biological sample using a competitive immunoassay. In a competitive immunoassay, the antigen in the biological sample competes with labeled antigen to bind with antibodies. The amount of labeled antigen bound to the antibody is then measured. In this method, the response will be inversely proportional to the concentration of antigen in the biological sample. This is because the greater the response, the less antigen in the unknown was available to compete with the labeled antigen.

In some embodiments, the biomarkers described herein are detected or measured in a biological sample using a noncompetitive immunoassay. In a noncompetitive immunoassay, also referred to as the “sandwich assay,” antigen in the biological sample is bound to a first antibody site, then a second antibody that is labeled is bound to the antigen, forming a sandwich. The amount of labeled antibody on the site is then measured. In contrast to the competitive immunoassay, the results of the noncompetitive immunoassay will be directly proportional to the concentration of the antigen.

Immunoassays are generally categorized into direct and indirect immunoassays. In a direct immunoassay, binding of antibody to the target antigen is determined directly. This direct assay uses a labeled reagent, such as a fluorescent tag or an enzyme-labeled primary antibody, which can be visualized without further antibody interaction. In an indirect assay, unconjugated primary antibody binds to the antigen and then a labeled secondary antibody binds to the primary antibody. The secondary antibody is conjugated to an enzymatic label and a chromogenic or fluorogenic substrate is added to provide visualization of the antigen. Signal amplification occurs because several secondary antibodies may react with different epitopes on the primary antibody.

In some embodiments, the primary and/or secondary antibody is labeled with a detectable moiety. Numerous labels are commercially available and well-known to those of skill in the art. Examples of detectable moieties include, but are not limited to, radioisotopes, colloidal gold particles, fluorescent labels, and enzyme-substrate labels.

In some embodiments, the detectable moiety is a radioisotope. Examples of radioisotopes include, but are not limited to, ³⁵S, ¹⁴C, ¹²⁵I, ³H, and ¹³¹I. An antibody can be labeled with the radioisotope using the techniques described in, e.g., Current Protocols in Immunology, Volumes 1 and 2, Coligen et al., Ed. Wiley-Interscience, New York, N.Y., Pubs. (1991) and radioactivity can be measured using scintillation counting.

In some embodiments, the detectable moiety is a colloidal gold particle.

In some embodiments, the detectable moiety is a fluorescent label. Fluorescent labels include, but are not limited to, rare earth chelates (europium chelates), Texas Red, rhodamine, fluorescein, dansyl, lissamine, umbelliferone, phycocrytherin, phycocyanin, or any other commercially available fluorophores or derivatives thereof. Fluorescence can be quantified using a fluorimeter. The fluorescent labels can be conjugated to the antibody using the techniques disclosed in, e.g., Current Protocols in Immunology, Volumes 1 and 2, Coligen et al., Ed. Wiley-Interscience, New York, N.Y., Pubs. (1991).

In some embodiments, the detectable moiety is an enzyme-substrate label as described in, e.g., U.S. Pat. No. 4,275,149. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase as described in, e.g., U.S. Pat. No. 4,737,456), luciferin, 2,3-dihydrophthalazinediones, malate dehydrogenase, urease, peroxidase (e.g., horseradish peroxidase (HRPO)), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (e.g., uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Numerous enzyme-substrate combinations are available to those skilled in the art. For a general review of enzyme-substrate combinations, See, e.g., U.S. Pat. Nos. 4,275,149 and 4,318,980. Examples of enzyme-substrate combinations include, for example, (a) horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); (b) alkaline phosphatase (AP) with para-Nitrophenyl phosphate as chromogenic substrate; and (c) μ-D-galactosidase (13-D-Gal) with a chromogenic substrate (e.g., p-nitrophenyl-β-D-galactosidase) or fluorogenic substrate (e.g., 4-methylumbelliferyl-β-D-galactosidase).

In some embodiments, the detection label is indirectly conjugated with the antibody. For example, the antibody can be conjugated with biotin and any of the four broad categories of labels mentioned above can be conjugated with avidin (or vice versa), which binds to biotin. In another example, the antibody is conjugated with a small hapten and one of the labels mentioned above is conjugated with an anti-hapten antibody. Thus, these labels are conjugated with the antibody in an indirect manner.

In some embodiments, the biomarkers described herein are detected or measured in a biological sample using a sandwich assay. Examples of sandwich assays include single-site and two-site sandwich assays of the non-competitive type, competitive binding assays, and direct binding assays of a labeled antibody to a biomarker. In a typical forward sandwich assay, an unlabeled antibody is immobilized on a solid substrate, and the biological sample to be tested is brought into contact with the bound molecule. After a period of time sufficient to allow formation of an antibody-antigen complex, a second antibody specific to the antigen and labeled with a reporter molecule capable of producing a detectable signal, is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of the biomarker. Variations on the forward sandwich assay include a simultaneous assay, in which both the biological sample and labeled antibody are added simultaneously to the bound antibody.

Another type of sandwich assay involves immobilizing the biomarker in the biological sample on a solid support and then exposing the immobilized biomarker to a specific antibody which may or may not be labeled with a reporter molecule. Depending on the amount of biomarker and the strength of the reporter molecule signal, the bound biomarker may be detectable by direct labelling with the antibody. Alternatively, a second labeled antibody, specific to the first antibody is exposed to the target-first antibody complex to form a target-first antibody-second antibody tertiary complex. The complex is detected by the signal emitted by the reporter molecule. The term “reporter molecule” as used herein refers to a molecule which, by its chemical nature, provides an analytically identifiable signal which allows the detection of the antigen-bound antibody. The most commonly used reporter molecules in this type of assay are either enzymes, fluorophores or radionuclide containing molecules (i.e. radioisotopes) and chemiluminescent molecules.

In some embodiments, the biomarkers described herein are detected or measured in a biological sample using an ELISA. Performing an ELISA involves at least one antibody with specificity for a particular biomarker. A known amount of anti-biomarker antibody is immobilized on a solid support either non-specifically (via adsorption to the surface) or specifically (via capture by another antibody specific to the anti-biomarker antibody, in a “sandwich” ELISA). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step, the plate is developed by adding an enzymatic substrate to produce a visible signal, which indicates the quantity of antigen in the sample. Older ELISAs utilize chromogenic substrates, though newer assays employ fluorogenic substrates with much higher sensitivity. Numerous ELISA methods and applications are known in the art and are further described in, e.g., Crowther, “Enzyme-Linked Immunosorbent Assay (ELISA),” in Molecular Biomethods Handbook, Rapley et al. (eds.), pp. 595-617, Humana Press, Inc., Totowa, N.J. (1998); Harlow and Lane (eds.), Antibodies: A Laboratory Manual, Cold Spring Harbor Laboratory Press (1988); Ausubel et al. (eds.), Current Protocols in Molecular Biology, Ch. 11, John Wiley & Sons, Inc., New York (1994).

In some embodiments, the biomarkers described herein are detected or measured in a biological sample using a direct ELISA. For example, in some embodiments, the biological sample containing the biomarker is exposed to a solid support. The biomarker within the biological sample becomes immobilized to the solid support, and is detected directly using an enzyme-conjugated antibody specific for the biomarker. A solution containing the appropriate enzyme substrate is then added to the antigen-antibody complex. The substrate will react with the enzyme linked to the antibody, giving a qualitative visual signal, which may be further quantitated, usually spectrophotometrically, to give an indication of the amount of biomarker which was present in the biological sample.

In some embodiments, the biomarkers described herein are detected or measured in a biological sample using an indirect ELISA. For example, in some embodiments, the biological sample containing the biomarker is exposed and immobilized to a solid support (e.g., a microtiter plate well). The biomarker is detected indirectly by first adding an antibody specific to the biomarker followed by the addition of a detection antibody specific for the antibody that specifically binds the biomarker. This detection antibody can be linked to an enzyme, and in the final step, a substance is added that the enzyme can convert to some detectable signal. For example, in the case of fluorescence ELISA, when light is shone upon the sample, any antigen/antibody complexes will fluoresce so that the amount of antibodies in the sample can be measured.

In some embodiments, the biomarkers described herein are detected or measured in a biological sample using a sandwich ELISA. For example, in some embodiments, a capture antibody specific to the biomarker is immobilized on a solid support (e.g., a microtiter plate) and a biological sample containing the biomarker is then added to the solid support. If the biomarker is present in the biological sample, it is bound to the capture antibody present on the solid support. In some embodiments, a sandwich ELISA is a “direct sandwich” ELISA, where the captured biomarker is detected directly by using an enzyme-conjugated antibody directed against the biomarker. Alternatively, in other embodiments, a sandwich ELISA is an “indirect sandwich” ELISA, where the captured biomarker is detected indirectly by using an antibody directed against the biomarker, which is then detected by another enzyme-conjugated antibody which binds the biomarker-specific antibody, thus forming an antibody-biomarker-antibody-antibody complex. Suitable reporter reagents are then added to detect the third antibody. In some embodiments, any number of additional antibodies are added as necessary, in order to detect the biomarker-antibody complex. In some embodiments, the additional antibodies are labeled or tagged, so as to permit their visualization and/or quantification of the biomarker.

In some embodiments, the biomarkers described herein are detected using an immunohistochemistry assay. Immunohistochemical staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Examples of methods used to visualize antibodies are, for example, through enzymes linked to the antibodies (e.g., luciferase, alkaline phosphatase, horseradish peroxidase, or β-galactosidase), or chemical methods (e.g., DAB/substrate chromagen). The sample is then analyzed microscopically, most preferably by light microscopy of a sample stained with a stain that is detected in the visible spectrum, using any of a variety of such staining methods and reagents known to those skilled in the art.

In some embodiments, the biomarkers described herein are detected or measured in a biological sample using a Western blot. The Western blot technique uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are detected using antibodies specific to the target protein. In some embodiments, the biomarkers are quantified by densitometry.

In some embodiments, the biomarkers described herein are detected or measured using a multiplex assay. A multiplex assay is a type of immunoassay that uses magnetic beads to simultaneously measure multiple analytes in a single experiment. In some embodiments, the multiplex assay is used to detect or measure phosphorylated and total endogenous protein, e.g., pSMAD2/3 and total SMAD2/3.

In some embodiments, the biomarkers described herein are detected using a lateral flow immunoassay test (LFIA), also known as the immunochromatographic assay, or strip test. LFIAs are a simple device intended to detect the presence or absence of a target antigen in a fluid sample. A LFIA test is a form of immunoassay in which the test sample flows along a solid substrate via capillary action. After the biological sample is applied to the test, it encounters a colored reagent which mixes with the sample and transits the substrate encountering the test lines or zones, which have been pretreated with an antibody or antigen. Depending upon the antigens present in the biological sample, the colored reagent can become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as serum, blood, and urine. LFIA strip test are easy to use, require minimum training and can be included as components of point-of-care test (POCT) diagnostics to be use on site in the field.

In some embodiments, the biomarkers described herein are immobilized on a solid support for detection in an immunoassay. In some embodiments, the antibody specific for the biomarker is immobilized on a solid support for detection of a biomarker in an immunoassay. The solid support is typically glass or a polymer, the most commonly used polymers being cellulose, polyacrylamide, nylon, polystyrene, polyvinyl chloride or polypropylene. In some embodiments, the solid support is in the form of a microplate, a tube, a bead, or any other surface suitable for conducting an immunoassay.

In some embodiments, the biomarkers described herein are detected or measured in a biological sample using a functional or activity-based assay. For example, if the biomarker is an enzyme, the expression level of the biomarker will be associated with the level of enzymatic activity in the biological sample.

### Kits

The present invention also provides kits adapted for the determination of the expression level of a biomarker in a biological sample of a subject with a disease or disorder, e.g., interstitial lung disease.

The term “kit” as used herein refers to a collection components assembled together with suitable packaging and instructions for their use.

In some embodiments, the kit comprises a container for the components of the kit. In some embodiments, the kit further comprises one or more additional containers comprising components of the kit. In some embodiments, the one or more additional containers comprise materials such as reagents (e.g., chromogen), buffers (e.g., blocking buffer, wash buffer, and substrate buffer), control samples, calibrators, diluents, filters, needles, and syringes.

In some embodiments, the kit comprises a container for holding or storing a biological sample (e.g., a container or cartridge for a sample). In some embodiments, the kit comprises one or more containers or reagents for preparing the biological sample. In some embodiments, the kit comprises one or more instruments for obtaining the biological sample, such as a syringe, pipette, forceps, measured spoon, or the like.

In some embodiments, the kit comprises reagents for the isolation and/or pre-treatment of a biological sample. For example, a whole blood sample obtained from a subject may be treated with a pre-treatment reagent prior to the assay used to detect the biomarker. Examples of pre-treatment reagents include, but are not limited to, solvents (e.g., methanol and ethylene glycol), salts, and detergents.

In some embodiments, the kit comprises an immunoassay for determining an expression level of a biomarker in a biological sample. In some embodiments, the kit comprises at least one capture reagent for the biomarker and at least one detection reagent for measurement of the biomarker. In some embodiments, the kit further comprises at least one capture reagent for a reference standard and detection reagents for measurement of the reference standard. In some embodiments, the kit detects the expression level of MYDGF, soluble RAGE, or pSMAD2/3. For example, in some embodiments, the kit comprises a monoclonal antibody specific for MYDGF. The monoclonal antibody directed to MYDGF is used to capture the MYDGF from a biological sample and then further conjugated antibodies are used to detect the presence of MYDGF that has been captured.

In some embodiments, the kit comprises a solid support. In some embodiments, antibodies specific for the biomarkers described herein are coated on the solid support. Examples of solid supports include, but are not limited to, microparticles, magnetic particles, beads, test tubes, microtiter plates, cuvettes, membranes, films, filter paper, discs or chips.

In some embodiments, the kit comprises quality control components. Examples of quality control components include, but are not limited to, sensitivity panels, calibrators, positive controls, and negative controls. Quality control components are used to establish assay performance characteristics, integrity of the kit reagents, and/or the standardization of assays. Calibrators are used to interpolate concentration of a biomarker in the biological sample. A positive control is a recombinant or biological sample that will be detectable in an assay. A negative control is a recombinant or biological sample that will not be detectable in an assay. In some embodiments, one or more of the reagents or components of the kit are lyophilized. In some embodiments, the kit further comprises one or more reagents (e.g., a buffer or water) for the reconstitution of the one or more lyophilized components.

### EXAMPLES

The disclosure is further described in detail by reference to the following examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### Example 1: Identification of Biomarkers in CSP-7-Treated IPF Fibroblasts and Epithelial Cells

This example describes the identification of biomarkers associated with CSP-7 treatment in fibroblasts and conditionally reprogrammed cells from subjects with IPF.

### Materials and Methods

### Samples and Cell Culture:

IPF fibroblasts and epithelial cells were used to identify potential biomarkers for CSP-7. Epithelial cells were derived from IPF lung explants using a modified conditional re-programming protocol (See, Liu et al., Am J Pathol, 2012; 180, 599-607; and Suprynowixz et al., PNAS, 2012; 109:20035-20040) and expanded in vitro. Epithelial cells are SSEA4⁺EpCAM⁺ basal progenitor cells that are similar to the pathogenic EpCAM+ basal-like cells found lining the honeycomb in IPF (See, Chilosi et al., Lab Invest, 2002; 82:1335-1345).

### CSP-7 Treatment:

IPF fibroblasts and epithelial cells were treated with 1% DMSO vehicle, 10 μM control peptide (CP), or 10 μM CSP-7 for 24 hours. Cells were then harvested and cell lysates were prepared using standard protocols.

### Protein Concentration:

Protein concentrations in the PCLS and epithelial cell samples were determined using a BCA assay. Briefly, equal amounts of protein extract was labeled using fluorescent dyes (size and charge matched). The spectrally resolvable dyes enabled simultaneous co-separation and analysis of protein concentration on a single multiplexed gel.

### 2D Gel Electrophoresis and Mass Spectrometry:

2D gels were prepped with isoelectric focusing (IEF) in the 1st dimension and SDS polyacrylamide gel electrophoresis (SDS-PAGE) in the 2nd dimension (2D-DIGE). After electrophoresis, the gels were scanned using a Typhoon image scanner. ImageQuant software was used to generate the image presentation data including the single and overlay images. Comparative analysis of all spots using DeCyder ‘in-gel’ or ‘cross-gel’ analysis software was performed. DMSO vehicle samples were pooled and used as reference samples. Protein expression ratios between CSP-7 or CP versus DMSO vehicle was determined, and p-values were assigned. Spots that were significantly regulated overall samples (P≤0.05) were assigned a number and automatically picked from the 2D gel with the Ettan Spot Picker. MALDI-TOF was then used to identify the spots.

ELISA: A commercial human MYDGF ELISA was used to quantitate MYDGF protein expression in the supernatants of CSP-7-treated IPF fibroblasts and epithelial cells and human plasma.

### Results:

Thirty-seven spots were significantly elevated in CSP-7-treated IPF fibroblasts and twenty-seven spots were significantly elevated in CSP-7-treated epithelial cells. The significantly regulated proteins upregulated in both CSP-7-treated IPF fibroblasts and epithelial cells are shown in Table 5 below.

TABLE 5
Proteins significantly regulated in CSP-7-treated IPF
fibroblasts (IPF Fibs) and epithelial cells (Epi cells)

| | ΔCSP-7/ | ΔCP/ | ΔCSP-7/ | ΔCP/ |
|---|---|---|---|---|
| Protein | DMSO | DMSO | DMSO | DMSO |
| Name | IPF Epi cells | IPF Epi cells | IPF Fibs | IPF Fibs |
| MYDGF | +1.4 | −1.0 | +1.5 | −1.2 |
| PROF1 | +1.4 | −1.7 | +1.9 | +1.1 |
| | +1.4 | −1.1 | +1.7 | +1.0 |
| CALM2 | +1.8 | +1.3 | +1.8 | +1.3 |
| | +1.4 | +1.3 | | |
| CALR | −2.5 | −2.3 | +1.4 | +1.0 |
| PPIA | +1.4 | +1.0 | +1.2 | −1.2 |
| | | | +1.5 | −1.0 |
| EIFA1 | +1.7 | +1.3 | +2.0 | +1.2 |

Delta signifies fold change in respective groups

Other significantly regulated proteins in CSP-7-treated IPF fibroblasts included galectin-1 (LGALS1), alpha-enolase (EnoA), and annexin V (ANXA5).

Other significantly regulated proteins in CSP-7-treated epithelial cells included fatty acid binding protein (FABP5), glutathione S-transferase P (GSTP-1), and 14-3-3 protein gamma.

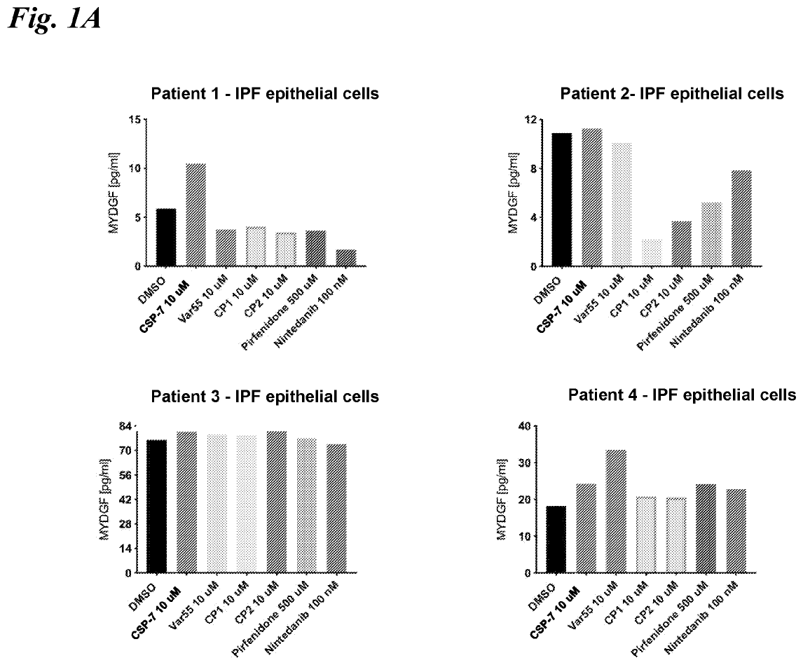

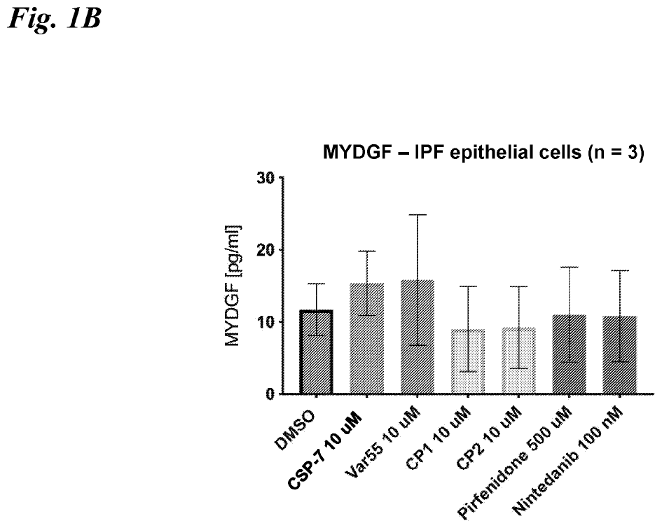

Additional experiments were performed to validate the MYDGF biomarker identified through MALDI-TOF. Epithelial cells derived from IPF lung explants were treated with 1% DMSO vehicle, CSP-7 (10 μM), Var55 (10 μM), control peptide 1 (CP1, 10 μM), control peptide 2 (CP2, 10 μM), pirfenidone (500 μM), or nintedanib (100 nM). MYDGF expression was measured in epithelial cell supernatant by ELISA. As shown in FIG. 1A and FIG. 1B, supernatants from epithelial cells treated with 10 μM or 100 μM of CSP-7 showed an increase in MYDGF protein.

Overall, these experiments identified a number of biomarkers induced in IPF fibroblasts and epithelial cells in response to CSP-7. Additional experiments confirmed that the MYDGF biomarker is increased in CSP-7-treated epithelial cells derived from patients with IPF. Healthy controls treated with CSP-7 experience no change in MYDGF protein expression.

### Example 2. Soluble RAGE as a Biomarker for CSP-7 Treatment

The endogenous secretory receptor for advanced glycation end products (esRAGE) is a soluble isoform produced by alternative splicing of the RAGE gene and is reduced in lung tissue, serum, and BAL of IPF patients with idiopathic pulmonary fibrosis (See, Yamaguchi et al., Respiratory Research, 2020; 21:145).

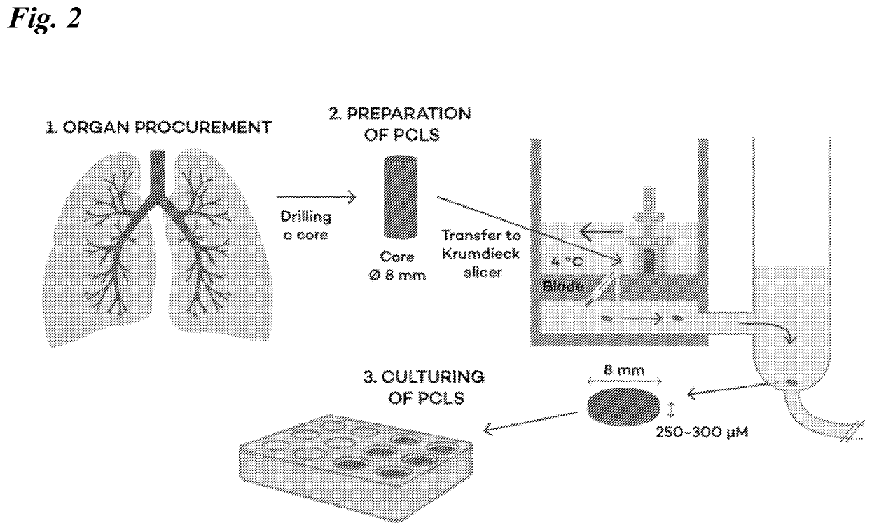

Precision cut lung slices (PCLSs) were used to determine whether soluble RAGE was affected by CSP-7 treatment. PCLSs enable testing of therapeutic agents, e.g., CSP-7, on human IPF lung tissue ex vivo (FIG. 2). Briefly, fresh lungs were obtained from human subjects with IPF and lung cores are drilled and transferred to a Krumdieck slicer. Precision cut lung slices (PCLS) were kept alive with human serum and culture media for up to one month.

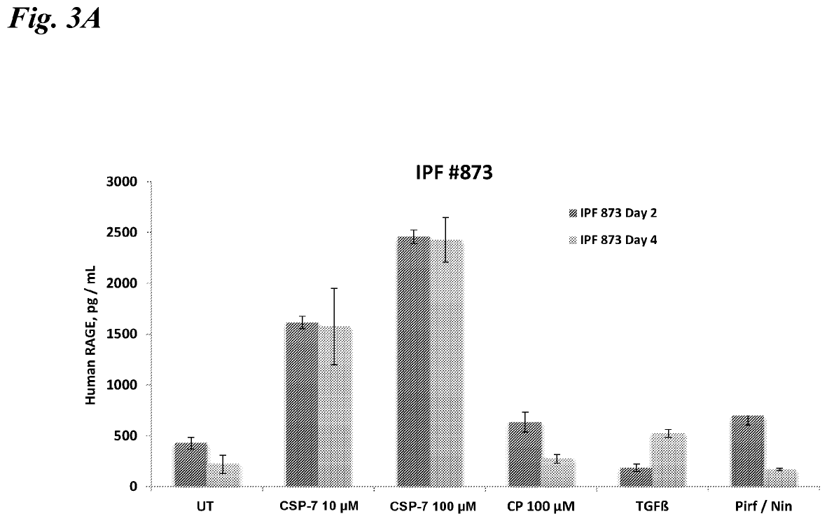

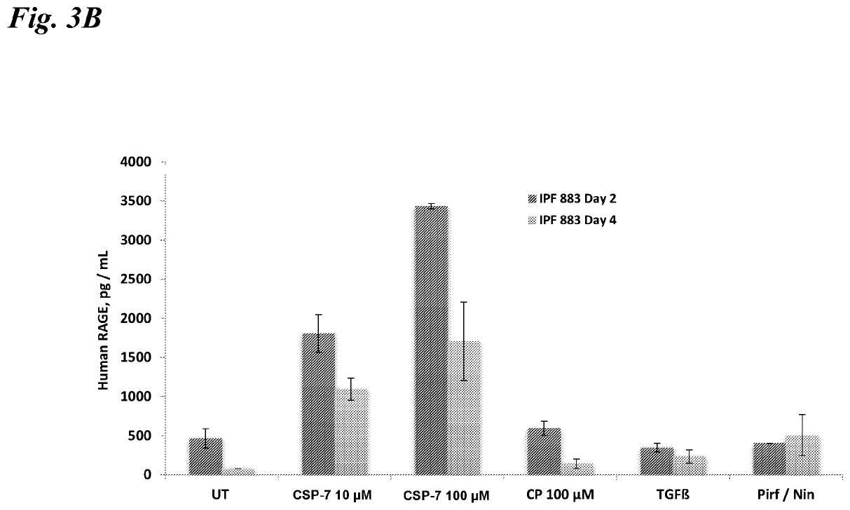

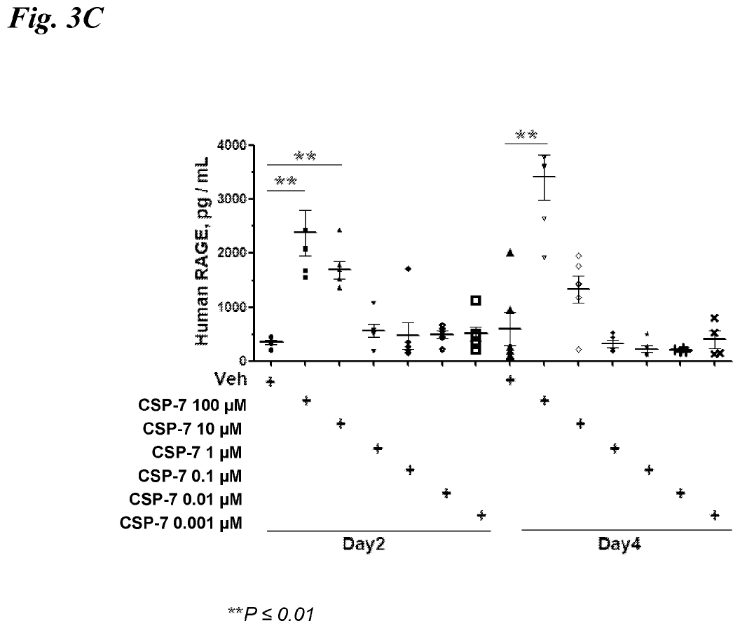

IPF PCLSs were either untreated (UT) or treated with CSP-7 (10 μM or 100 μM), control peptide (CP, 100 μM), TGFβ (2 ng/mL), or pirfenidone (Pirf, 500 μM) and nintedanib (Nin, 100 nM). Soluble RAGE expression was measured in the supernatant of IPF PCLSs on day 2 and 4 following treatment by ELISA (R&D Systems, Cat No. DRG00). The ELISA detects the extracellular domain of human RAGE. As shown in FIG. 3A and FIG. 3B, IPF PCLSs treated with either 10 μM or 100 μM of CSP-7 exhibited an increase in soluble RAGE compared to untreated or CP-treated controls. FIG. 3C shows a dose-dependent increase in soluble RAGE expression in IPF PCLSs on days 2 and 4 following treatment with CSP-7.

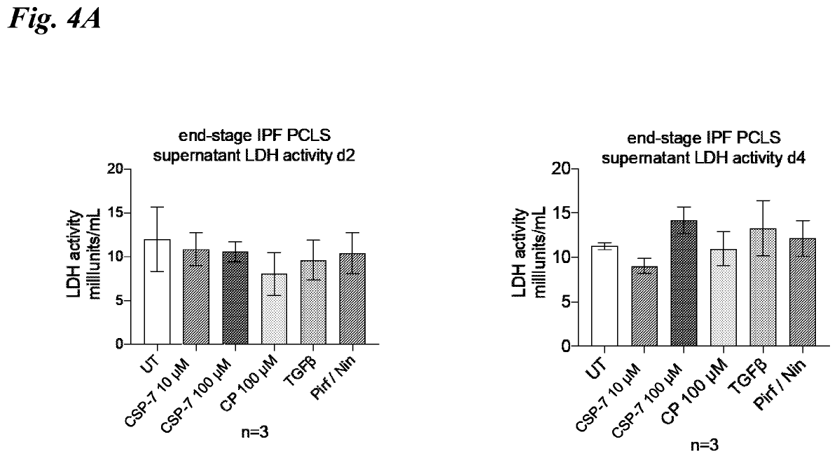

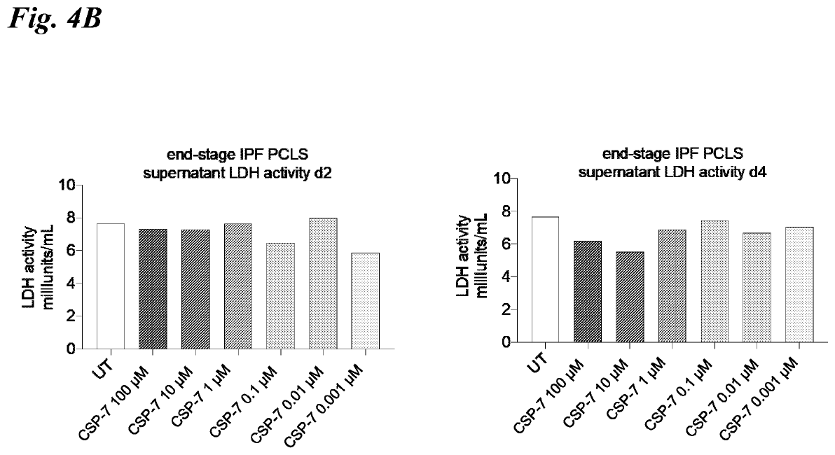

LDH activity was also examined in the supernatants of IPF PCLSs either untreated (UT) or treated with CSP-7 (10 μM or 100 μM), control peptide (CP, 100 μM), TGFβ (2 ng/mL), or pirfenidone (Pirf, 500 μM) and nintedanib (Nin, 100 nM). LDH activity was measured using an LDH assay (Sigma-Aldrich, Cat No MAK066). Overall, there was a slight reduction in LDH activity in CSP-7-treated PCLSs compared to untreated controls (FIG. 4A and FIG. 4B).

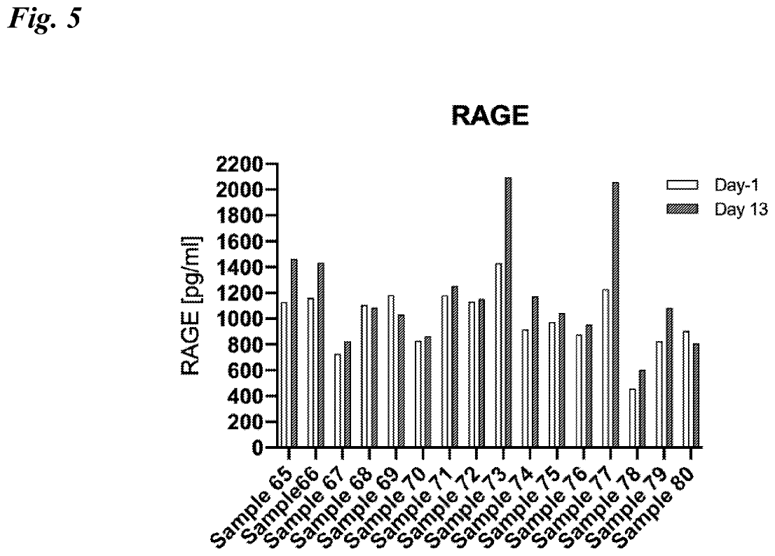

Soluble RAGE expression was also examined in healthy subjects on day 1 and day 13 following inhalation of CSP-7 peptide. As shown in FIG. 5, soluble RAGE expression in plasma of CSP-7-treated healthy subjects was elevated on day 1 and 13 following treatment with CSP-7.

Overall, these data demonstrate increased expression of soluble RAGE in end-stage IPF PCLS cultures following treatment with CSP-7. The reduction in LDH activity in CSP-7-PCLSs may also indicate healthier explant tissue and increased epithelial cell survival, which is critical to epithelial repair and reduced fibrosis.

### Example 3. PDGFRβ as a Biomarker for CSP-7 Therapy

Platelet-derived growth factor (PDGF) signaling has been implicated in the pathogenesis of pulmonary fibrosis. A number of fibrogenic mediators such as TGF-β, IL-1, TNF-α, bFGF, and thrombin exhibit PDGF-dependent profibrotic activities. Inhibition of PDGF signaling has also been shown to ameliorate bleomycin-induced pulmonary fibrosis in mice. See, Abdollahi et al., JEM, 2005 201 (6): 925-935; and Kishi et al., PLOS One, 2018, 13 (12): e0209786).

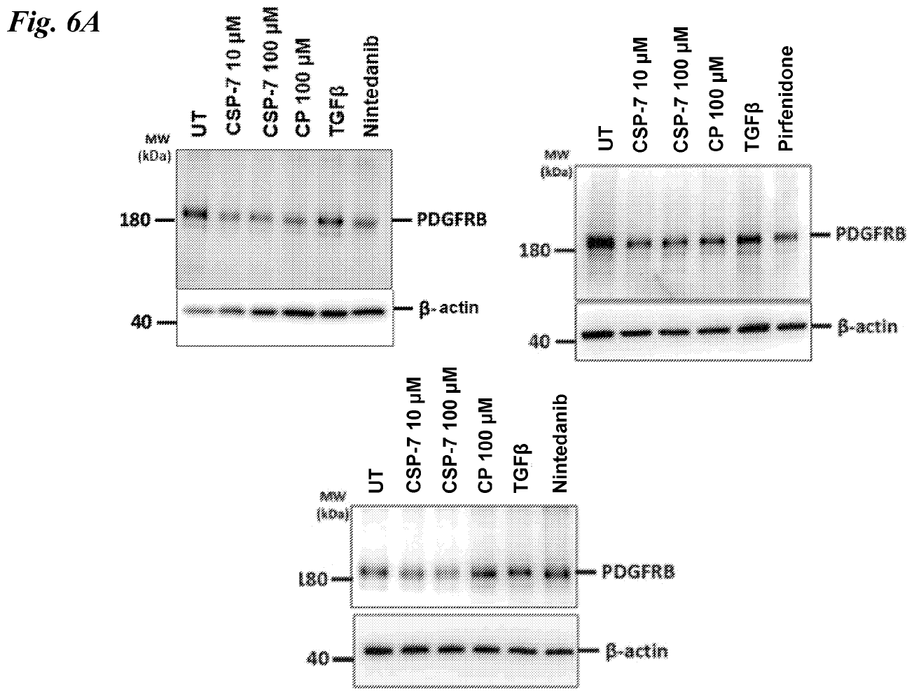

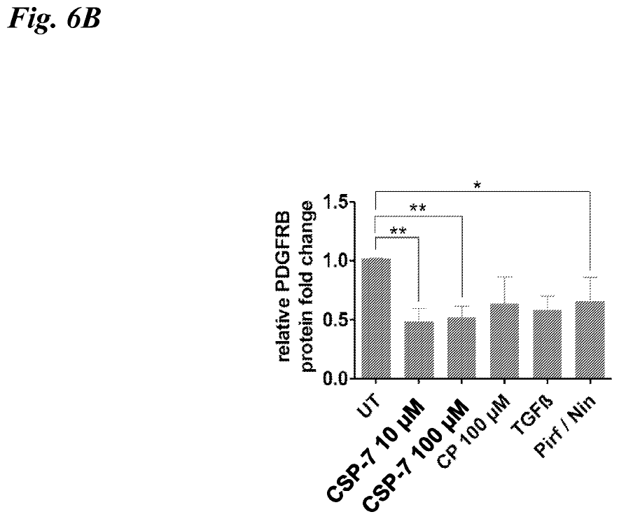

Experiments were performed to determine whether PDGFRβ could be used as a biomarker for CSP-7. PCLSs were obtained from IPF subjects and cultured as described above and shown in FIG. 2. PCLSs were either untreated (UT) or treated with CSP-7 (10 μM or 100 μM), control peptide (CP, 100 μM), TGFβ (2 ng/ml), or pirfenidone (Pirf, 500 μM) and nintedanib (Nin, 100 nM). PDGFRβ expression was measured in the supernatant of IPF PCLSs on day 2 and 4 following treatment by ELISA (R&D Systems, Cat No. DRG00). As shown in FIG. 6A and FIG. 6B, PCLSs treated with either 10 UM or 100 μM of CSP-7 exhibited a decrease in PDGFRβ protein expression compared to untreated controls.

Overall, these data demonstrate reduced expression of PDGFRβ in end-stage IPF PCLS cultures following treatment with CSP-7. PDGFRβ may be a useful biomarker associated with CSP-7 therapy in patients with fibrosis.

### Example 4. Phosphorylated SMAD2/3 as a Biomarker for CSP-7 and Var55 Therapy

TGF-β plays a central role in fibrosis, contributing to the influx and activation of inflammatory cells, the epithelial to mesenchymal transdifferentiation (EMT) of cells and the influx of fibroblasts and their subsequent elaboration of extracellular matrix. TGF-β signals through SMAD proteins such as SMAD2/3. Experiments were performed to determine whether the phosphorylation status of SMAD2/3 could be used as a biomarker associated with CSP-7 and/or Var55 treatment.

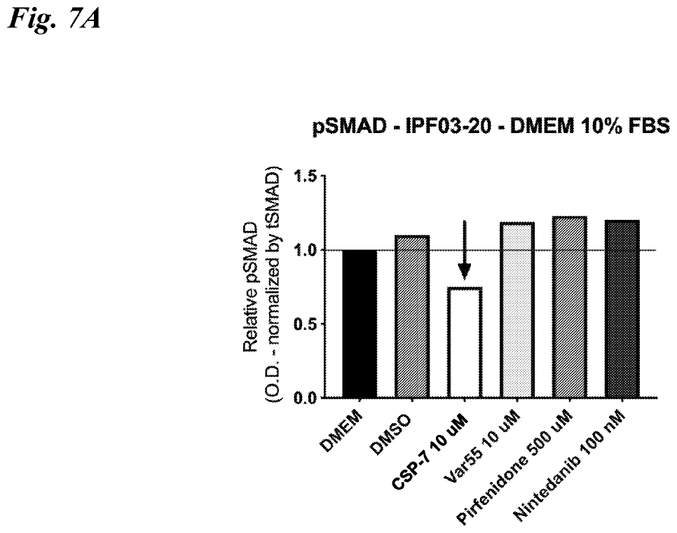

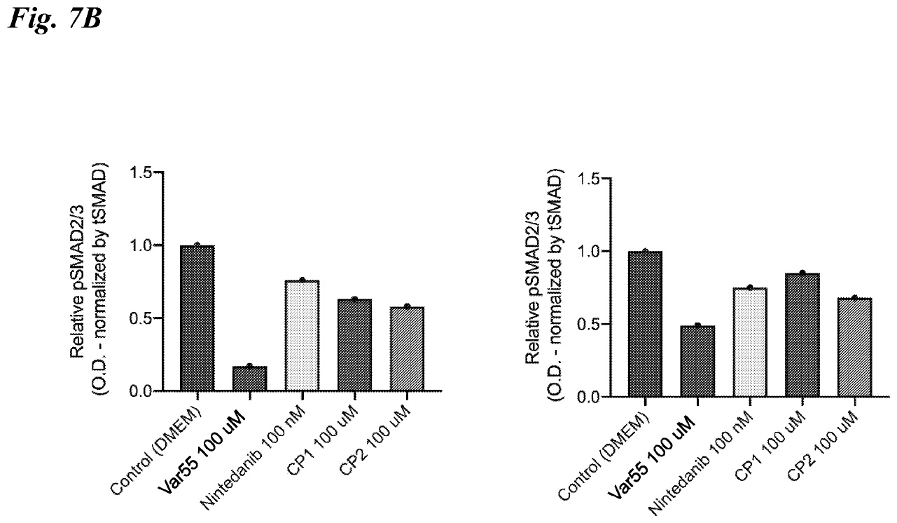

Primary lung fibroblasts from IPF patients were treated with 1% DMSO vehicle, CSP-7 (10 μM), Var55 (10 μM), pirfenidone (500 μM), or nintedanib (100 nM). SMAD2/3 phosphorylation was measured in IPF fibroblasts by a multiplex assay or Western blot analysis and phosphorylated SMAD2/3 was normalized to total SMAD2/3 protein. As shown in FIG. 7A, IPF fibroblasts treated with 10 μM of CSP-7 exhibited reduced phosphorylation of SMAD2/3 compared to 1% DMSO vehicle or DMEM controls. IPF fibroblasts treated with 10 μM of Var55 showed no difference in SMAD2/3 phosphorylation.

Additional experiments were performed to determine whether phosphorylation of SMAD2/3 could be used as a biomarker associated with Var55 treatment. Epithelial cells from IPF patients were treated with DMEM (control), Var55 (100 μM), nintedanib (100 nM), control peptide 1 (CP1, 100 μM), or control peptide 2 (CP2, 100 μM). SMAD2/3 phosphorylation was measured in IPF fibroblasts by a multiplex assay or Western blot analysis and phosphorylated SMAD2/3 was normalized to total SMAD2/3 protein. As shown in FIG. 7B, IPF epithelial cells treated with 100 μM of Var55 exhibited reduced phosphorylation of SMAD2/3 compared to DMEM control.

Collectively, these data demonstrate reduced SMAD2/3 phosphorylation in IPF fibroblasts and epithelial cells following treatment with CSP-7 and Var55. SMAD2/3 phosphorylation may therefore be a useful biomarker associated with CSP-7 and/or Var55 treatment in patients with fibrosis.

### Example 5. Galectin-7 as a Biomarker for CSP-7 and Var55 Therapy

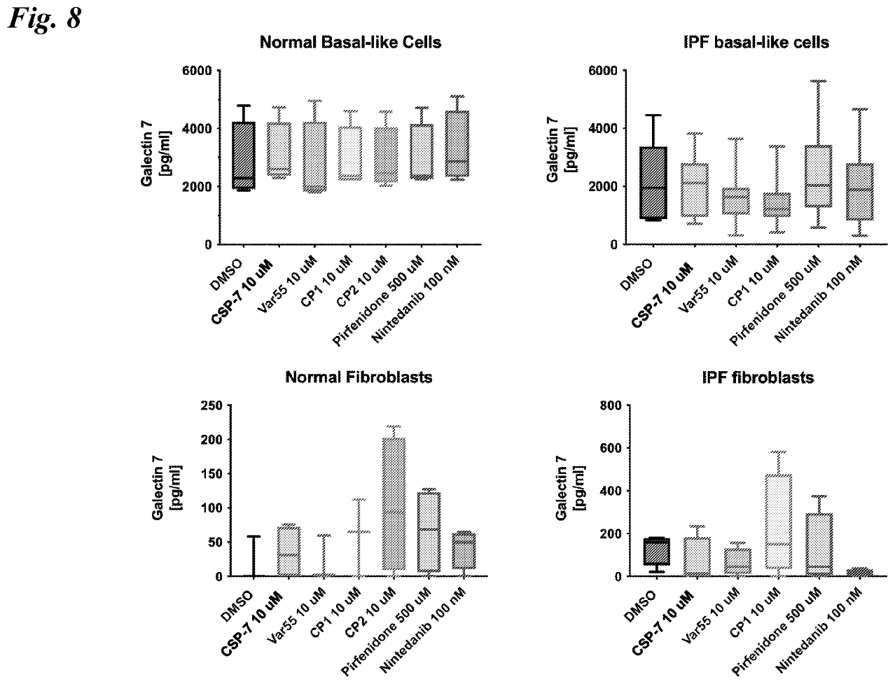

Basal-like cells and fibroblasts derived from either normal healthy subjects or subjects with idiopathic pulmonary fibrosis (IPF) were treated with DMSO vehicle, CSP-7 (10 μM), Var55 (10 μM), control peptide 1 (CP1, 10 μM), control peptide 2 (CP2, 10 μM), pirfenidone (500 μM), or nintedanib (100 nM). Galectin-7 was measured by ELISA. The results of the study are shown in FIG. 8.

### Example 6. Biomarkers of Modified Cav-1 Peptides in an Aged Mouse Model of Lung Fibrosis

The objective of this study was to evaluate the modified Cav-1 peptide CSP-7 in a bleomycin model of idiopathic pulmonary fibrosis and acute respiratory distress syndrome (ARDS). Aged male mice were selected to closely match the population of humans most vulnerable to ARDS.

Bleomycin (BLM, 8 U/kg) was administered intratracheally to 73-74 week-old mice (n=52) expecting approximately 50% loss of mice due to BLM-induced lung injury. On day 14 post-instillation, CSP-7 and a control peptide (CP) was administered to mice via dry powder inhalation (DPI) or intraperitoneal injection (IP) as shown in Table 6 below. On day 21 post-instillation, mice were sacrificed and blood and tissues were collected for analysis. Statistics were performed using Dunnett's multiple comparison test.

TABLE 6

| | | BLM Dose | Treat- | | | |
|---|---|---|---|---|---|---|
| Group | Description | (U/kg) | ment | Dose | Route | n |
| A | BLM CSP-7 | 8 | CSP-7 | 0.5 mg/mouse | DPI | 8 |
| | DPI | | | | | |
| D | BLM CP DPI | 8 | Control | 0.5 mg/mouse | DPI | 8 |
| | | | Peptide | | | |
| E | BLM CSP-7 IP | 8 | CSP-7 | 1.5 mg/kg | IP | 6 |
| B | BLM control | 8 | None | None | — | 8 |
| C | Saline control | Saline | None | None | — | 8 |
| | | control | | | | |

| | |
|---|---|
| Total Animals | 38 |

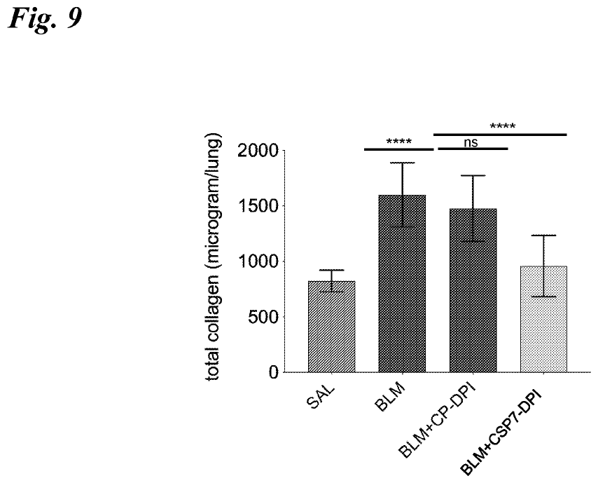

FIG. 9 shows collagen in total lung homogenates of saline or BLM-treated aged mice administered control peptide (CP) or CSP-7 through dry powder inhalation (DPI). BLM-treated mice administered CSP-7 showed a reduction in total collagen in the lung compared to mice administered control peptide.

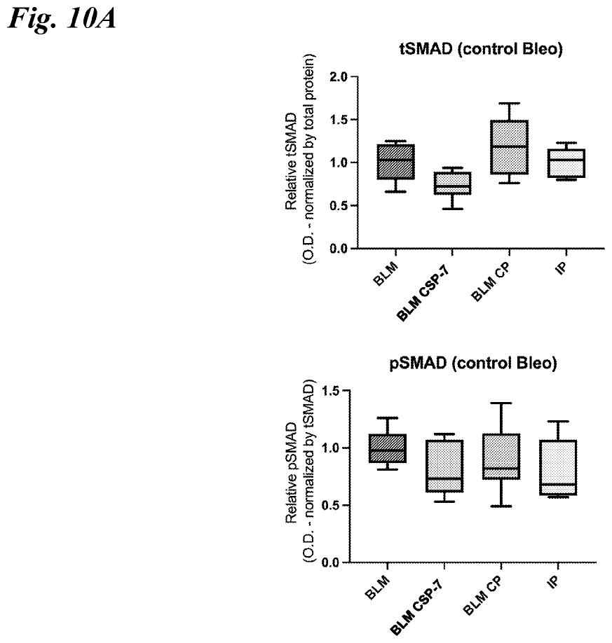

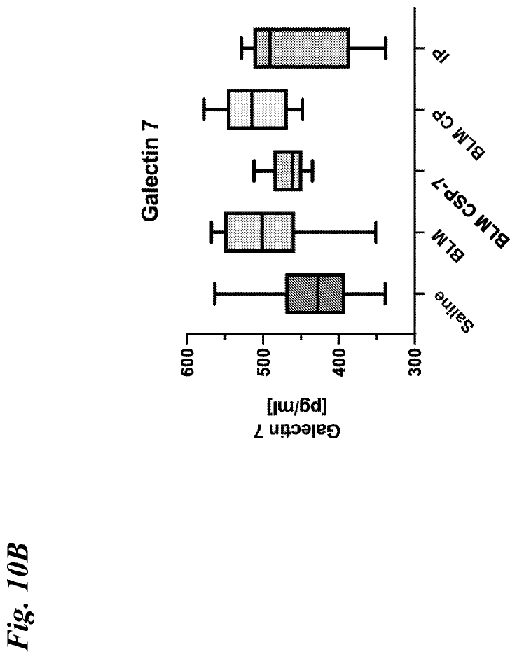

FIG. 10A shows total SMAD (tSMAD, top panel) and phosphorylated SMAD (pSMAD, bottom panel) in total lung homogenates of BLM-treated aged mice administered control peptide (CP) or CSP-7 through dry powder inhalation (DPI) or CSP-7 through intraperitoneal injection (IP).

FIG. 10B shows galectin-7 in total lung homogenates of BLM-treated aged mice administered control peptide (CP) or CSP-7 through dry powder inhalation (DPI) or CSP-7 through intraperitoneal injection (IP).

### Example 7. CSP-7 Attenuates Receptor Tyrosine Kinase Signaling in IPFs

The objective of this study was to examine the effect of CSP-7 treatment on receptor tyrosine kinase signaling in IPF fibroblasts.

End-stage IPF fibroblasts and non-cancerous donor lung fibroblasts were obtained and cultured in vitro. Early passages from normal donor and IPF fibroblasts were either left untreated or treated with DMSO, control peptide, or 10 μM CSP-7 for 24 hours. Cells were then harvested for a reverse phase protein array. The array contained 240 validated antibodies against total proteins and phosphoproteins, epithelial-mesenchymal transition (EMT), stem cells, apoptosis, DNA damage, autophagy, proliferation and cell cycle, growth factor receptors, cytokines/STATs, nuclear receptors/transcriptional regulatory proteins, autophagy, and metabolomic enzymes. Nine technical replicates were run for each sample, and signals were normalized to total protein.

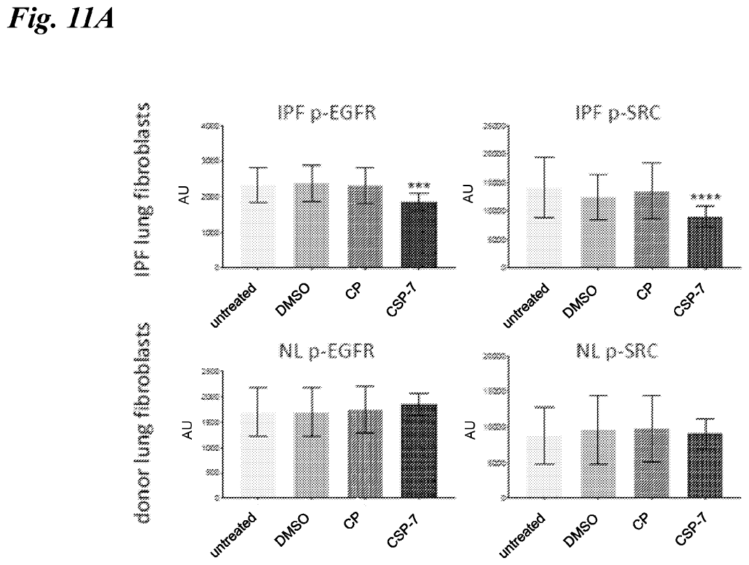

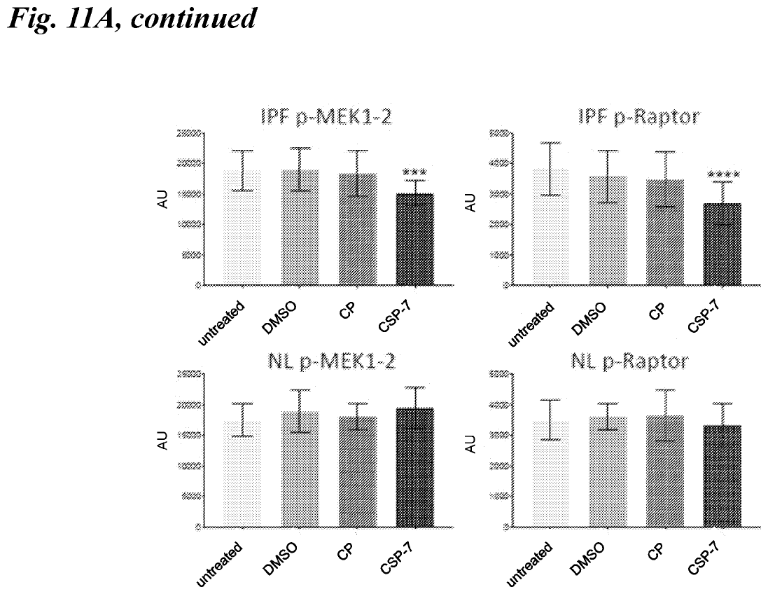

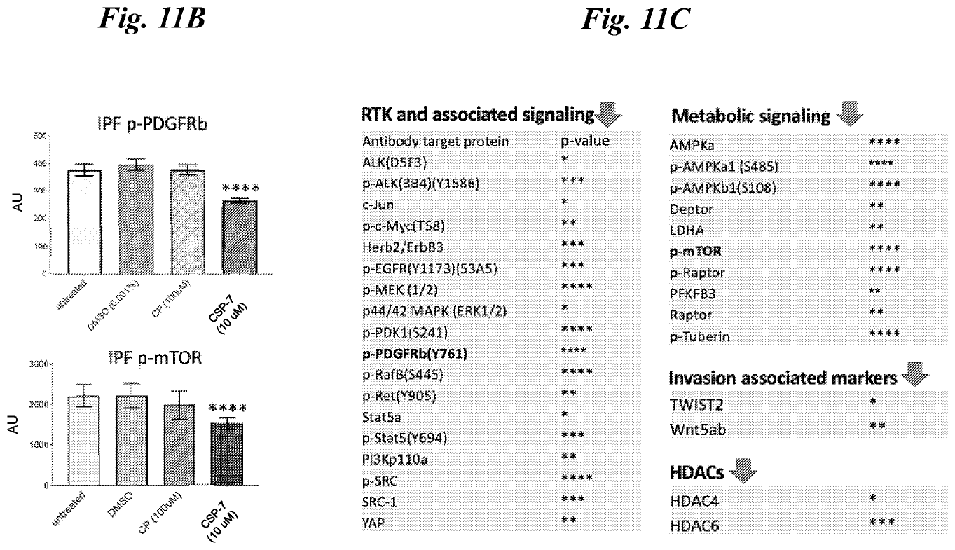

FIG. 11A shows that IPFs treated with CSP-7 showed reduced phosphorylation of EGFR, SRC, MEK1/2, and Raptor while CSP-7 treatment of normal lung fibroblasts had no effect on these signaling pathways.

FIG. 11B shows that IPFs treated with CSP-7 showed suppression of p-PDGFRβ and p-mTOR compared to untreated IPFs or those treated with DMSO or control peptide.

FIG. 11C provides a summary of the reverse phase protein array that assessed receptor tyrosine kinase (RTK) signaling, metabolic signaling, invasion-associated markers, and histone deacetylases (HDAC).

Overall, these results indicate that CSP-7 treatment of IPFs reduces activation of receptor tyrosine kinase signaling, metabolic signaling, invasion-associated markers, and histone deacetylases (HDACs). Of particular interest is the suppression of p-PDGFRβ as p-mTOR signaling shown in FIG. 11B, which is known to contribute to supporting myofibroblasts. These results suggest that CSP-7 may act as sponge of aberrant signaling when administered to IPFs while having limited effects on fibroblasts with normal signaling.

### Example 8. IL-11 as a Biomarker for Modified Cav-1 Peptide Therapy

The objective of this study was to assess the effect of CSP-7 treatment on IL-11 expression in the lungs of mice with acute lung injury.

Mice were treated with bleomycin to induce acute lung injury. Mice treated with saline only were used as a negative control. Mice were assigned to either a preventative or therapeutic treatment group. The preventative treatment group received three daily doses of control peptide or CSP-7 prior to administration of bleomycin. The therapeutic treatment group received three daily doses of control peptide or CSP-7 after administration of bleomycin. Mice were sacrificed on day 4 following treatment with bleomycin and bronchoalveolar lavage fluid (BALF) was collected from mice for measurement of IL-11.

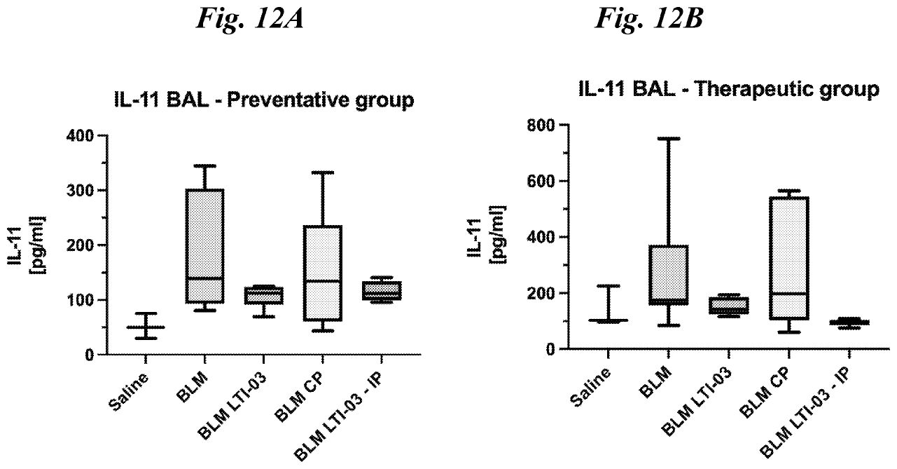

As shown in FIG. 12A, bleomycin-treated mice in the preventative treatment group administered CSP-7 showed a reduction in IL-11 levels in the BALF compared to bleomycin-treated mice administered control peptide. As shown in FIG. 12B, bleomycin-treated mice in the therapeutic treatment group administered CSP-7 showed a reduction in IL-11 levels in the BALF compared to bleomycin-treated mice administered with control peptide.

### Example 9. sCD163 and MMP-2 as Biomarkers for Modified Cav-1 Peptide Therapy

The objective of this study was to assess the effect of CSP-7 treatment on sCD163 and MMP-2 expression on lung macrophages from humans with IPF.

Lung macrophages were collected from humans with IPF and cultured in vitro. Lung macrophages were either left untreated or treated with 0.1 μM or 1 μM of CSP-7. One day after treatment, supernatants from the lung macrophage cultures were collected and analyzed for sCD163 and MMP-2.

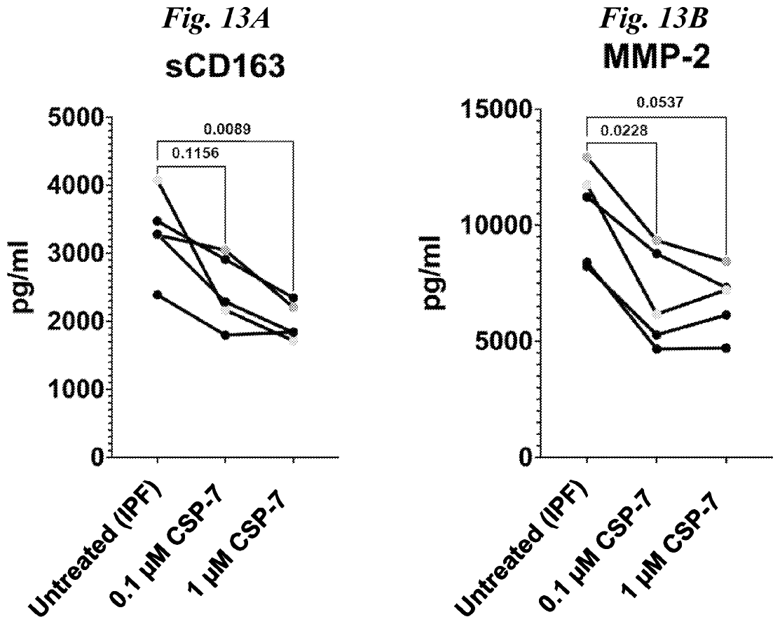

As shown in FIG. 13A, IPF macrophages treated with CSP-7 showed a reduction in sCD163 in the supernatant compared to untreated controls. As shown in FIG. 13B, IPF macrophages treated with CSP-7 showed a reduction in MMP-2 in the supernatant compared to untreated controls.

### Example 10. CXCL7 as a Biomarker for Modified Cav-1 Peptide Therapy

The objective of this study was to assess the effect of Var55 treatment on CXCL7 expression on lung macrophages from humans with IPF.

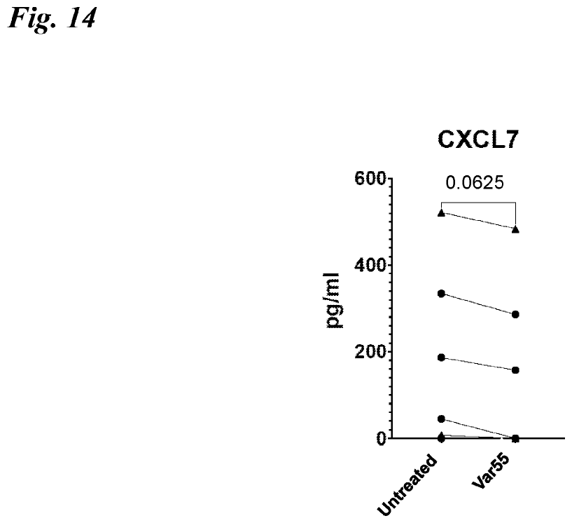

Lung macrophages were collected from humans with IPF or healthy controls and cultured in vitro. Lung macrophages were either left untreated or treated with Var55. Following treatment, supernatants from the lung macrophage cultures were collected and analyzed for CXCL7 expression by ELISA. As shown in FIG. 14, healthy controls (triangles) and IPF macrophages (circles) treated with Var55 showed a significant reduction in CXCL7 in the supernatant.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method for treating a subject with caveolin-1 peptide therapy, wherein the subject is suffering from fibrosis, the method comprising the steps of:
(a) obtaining or having obtained a biological sample from the subject;
(b) treating or having treated a cell of the biological sample with a caveolin-1 peptide or derivative thereof;
(c) measuring an expression level of a biomarker in the cell; and
(d) comparing the expression level of the biomarker to a control sample;
wherein the biomarker is myeloid-derived growth factor (MYDGF), soluble RAGE, phosphorylated mothers against decapentaplegic homolog 2/3 (pSMAD2/3), platelet-derived growth factor receptor beta (PDGFRβ), galectin-7 (LGALS7), interleukin-11 (IL-11), matrix metalloproteinase-2 (MMP-2), chemokine ligand 7 (CXCL-7), soluble CD163, phosphorylated mTOR (p-mTOR), phosphorylated PDGFRβ (pPDGFRβ), prolifin (PROF1), calmodulin 2 (CALM2), calreticulin (CALR), peptidyl-prolyl cis-trans isomerase A (PPIA), or eukaryotic translation initiation factor 5A (EIF5A1); and
if the expression level of MYDGF, PROF1, CALM2, CALR, PPIA, IF5A1, LGALS7, soluble RAGE, or EIF5A1 is increased, then administering caveolin-1 peptide or derivative thereof to the subject; or
if the expression level of PDGFRβ, pPDGFRβ, p-mTOR, CALR, IL-11, MMP-2, CXCL7, soluble CD163, or pSMAD2/3 is decreased, then administering caveolin-1 peptide or derivative thereof to the subject.

2. The method of claim 1, wherein the control sample is a cell obtained from the subject prior to treatment with the caveolin-1 peptide or derivative thereof.

3. The method of claim 1, wherein the control sample is a cell obtained from a subject or a population of subjects with interstitial lung disease.

4. The method of claim 1, wherein the control sample is a cell obtained from a healthy subject.

5. The method of any one of claims 1-4, wherein the cell is a fibroblast, a type I alveolar epithelial cell, a type II alveolar epithelial cell, a basal-like cell, a Clara cell, a ciliated cell, a club cell, a goblet cell, a neuroendocrine cell, an endothelial cell, a bialveolar stem cell, a macrophage, an alveolar macrophage, an ionocyte, a pericyte, a mesothelial cell, a mesenchymal cell, a neuroendocrine cell, a myofibroblast, a B-cell, a plasma cell, an innate lymphoid cell, a T-cell, a monocyte, an NK cell, a dendritic cell, or a peripheral blood mononuclear cell (PBMC).

6. The method of claim 5, wherein the cell is a fibroblast.

7. The method of claim 5, wherein the cell is a basal-like cell.

8. The method of any one of claims 1-7, wherein the cell is treated with the caveolin-1 peptide or derivative thereof ex vivo or in vitro.

9. The method of any one of claims 1-8, wherein the caveolin-1 peptide or derivative thereof is FTTFTVT (SEQ ID NO: 3).

10. The method of any one of claims 1-9, wherein the caveolin-1 peptide or derivative thereof is Ac-aaEGKASFTTFTVTKGSaa-NH2 (SEQ ID NO: 4).

11. The method of any one of claims 1-10, wherein fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

12. The method of claim 11, wherein the interstitial lung disease is idiopathic pulmonary fibrosis, lymphangioleiomyomatosis, nonspecific interstitial pneumonia, idiopathic interstitial pneumonia, cryptogenic organizing pneumonia, acute interstitial pneumonia, respiratory bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease.

13. The method of claim 12, wherein the interstitial lung disease is idiopathic pulmonary fibrosis.

14. The method of any one of claims 1-13, wherein the expression level of the biomarker is measured by 2-D gel electrophoresis, Western blot, mass spectrometry, flow cytometry, quantitative RT-PCR, ELISA, and/or a lateral flow immunoassay.

15. The method of any one of claims 1-14, wherein administering caveolin-1 peptide or derivative thereof to the subject if the expression level of MYDGF is increased compared to the control sample.

16. The method of claim 15, wherein the expression level of MYDGF is increased by about 1.5-fold, about 2-fold, about 3-fold, about 4-fold, about 5-fold, or more compared to the control sample.

17. The method of any one of claims 1-14, wherein administering caveolin-1 peptide or derivative thereof to the subject if the expression level of soluble RAGE is increased compared to the control sample.

18. The method of claim 17, wherein the expression level of soluble RAGE is increased by about 1.5-fold, about 2-fold, about 3-fold, about 4-fold, about 5-fold, or more compared to the control sample.

19. The method of any one of claims 1-14, wherein administering caveolin-1 peptide or derivative thereof to the subject if the expression level of pSMAD2/3 is decreased compared to the control sample.

20. The method of claim 19, wherein the expression level of pSMAD2/3 is decreased by about 10%, about 25%, about 50%, about 75%, about 90%, or more compared to the control sample.

21. The method of any one of claims 1-14, wherein administering caveolin-1 peptide or derivative thereof to the subject if the expression level of PDGFRβ and/or pPDGFRβ is decreased compared to the control sample.

22. The method of claim 21, wherein the expression level of PDGFRβ and/or pPDGFRβ is decreased by about 10%, about 25%, about 50%, about 75%, about 90%, or more compared to the control sample.

23. The method of any one of claims 1-22, wherein the method further comprises measuring an expression level of an internal control and wherein the expression level of the internal control is unaffected by the caveolin-1 peptide or derivative thereof.

24. The method of any one of claims 1-23, wherein steps (b)-(d) of the method are repeated one or more times.

25. A method of identifying an altered expression level of a biomarker associated with caveolin-1 peptide therapy, the method comprising:
(a) treating a cell of a subject with interstitial lung disease with a caveolin-1 peptide or derivative thereof;
(b) measuring an expression level of a biomarker in the cell; and
(c) comparing the expression level of the biomarker to a control sample;
wherein the caveolin-1 peptide or derivative thereof modulates the expression level of the biomarker in the cell; and
wherein the biomarker is myeloid-derived growth factor (MYDGF), soluble RAGE, phosphorylated mothers against decapentaplegic homolog 2/3 (pSMAD2/3), platelet-derived growth factor receptor beta (PDGFRβ), galectin-7 (LGALS7), interleukin-11 (IL-11), matrix metalloproteinase-2 (MMP-2), chemokine ligand 7 (CXCL-7), soluble CD163, phosphorylated mTOR (p-mTOR), phosphorylated PDGFRβ (pPDGFRβ), prolifin (PROF1), calmodulin 2 (CALM2), calreticulin (CALR), peptidyl-prolyl cis-trans isomerase A (PPIA), or eukaryotic translation initiation factor 5A (EIF5A1).

26. The method of claim 25, wherein the control sample is a cell obtained from the subject with interstitial lung disease prior to treatment with the caveolin-1 peptide or derivative thereof.

27. The method of claim 25, wherein the control sample is a cell obtained from a subject or a population of subjects with interstitial lung disease.

28. The method of claim 25, wherein the control sample is a cell obtained from a healthy subject.

29. The method of any one of claims 25-28, wherein the cell is a fibroblast, a type I alveolar epithelial cell, a type II alveolar epithelial cell, a basal-like cell, a Clara cell, a ciliated cell, a club cell, a goblet cell, a neuroendocrine cell, an endothelial cell, a bialveolar stem cell, a macrophage, an alveolar macrophage, an ionocyte, a pericyte, a mesothelial cell, a mesenchymal cell, a neuroendocrine cell, a myofibroblast, a B-cell, a plasma cell, an innate lymphoid cell, a T-cell, a monocyte, an NK cell, a dendritic cell, or a peripheral blood mononuclear cell (PBMC).

30. The method of claim 29, wherein the cell is a fibroblast.

31. The method of claim 29, wherein the cell is a basal-like cell.

32. The method of any one of claims 25-31, wherein the cell is treated with the caveolin-1 peptide or derivative thereof ex vivo or in vitro.

33. The method of claim 32, wherein the cell is obtained from the subject with interstitial lung disease.

34. The method of any one of claims 25-33, wherein the caveolin-1 peptide or derivative thereof is FTTFTVT (SEQ ID NO: 3).

35. The method of any one of claims 25-33, wherein the caveolin-1 peptide or derivative thereof is Ac-aaEGKASFTTFTVTKGSaa-NH2 (SEQ ID NO: 4).

36. The method of any one of claims 25-35, wherein the subject is a human.

37. The method of any one of claims 25-36, wherein the interstitial lung disease is idiopathic pulmonary fibrosis, lymphangioleiomyomatosis, nonspecific interstitial pneumonia, idiopathic interstitial pneumonia, cryptogenic organizing pneumonia, acute interstitial pneumonia, respiratory bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease.

38. The method of claim 37, wherein the interstitial lung disease is idiopathic pulmonary fibrosis.

39. The method of any one of claims 25-38, wherein the expression level of the biomarker is measured by 2-D gel electrophoresis, Western blot, mass spectrometry, flow cytometry, quantitative RT-PCR, ELISA, and/or a lateral flow immunoassay.

40. The method of any one of claims 25-39, wherein the increased expression level of MYDGF, PROF1, CALM2, CALR, PPIA, IF5A1, LGALS7, soluble RAGE, or EIF5A1 is associated with caveolin-1 therapy.

41. The method of any one of claims 25-40, wherein the decreased expression level of PDGFRβ, pPDGFRβ, p-mTOR, CALR, IL-11, MMP-2, CXCL7, sCD163, or pSMAD2/3 is associated with caveolin-1 therapy.

42. The method of any one of claims 25-40, wherein the expression level of MYDGF is increased compared to the control sample.

43. The method of claim 42, wherein the expression level of MYDGF is increased by about 1.5-fold, about 2-fold, about 3-fold, about 4-fold, about 5-fold, or more compared to the control sample.

44. The method of any one of claims 25-40, wherein the expression level of soluble RAGE is increased compared to the control sample.

45. The method of claim 44, wherein the expression level of soluble RAGE is increased by about 1.5-fold, about 2-fold, about 3-fold, about 4-fold, about 5-fold, or more compared to the control sample.

46. The method of any one of claims 25-40, wherein the expression level of pSMAD2/3 is decreased compared to the control sample.

47. The method of claim 46, wherein the expression level of pSMAD2/3 is decreased by about 10%, about 25%, about 50%, about 75%, about 90%, or more compared to the control sample.

48. The method of any one of claims 25-40, wherein the expression level of PDGFRβ and/or pPDGFRβ is decreased compared to the control sample.

49. The method of claim 48, wherein the expression level of PDGFRβ and/or pPDGFRβ is decreased by about 10%, about 25%, about 50%, about 75%, about 90%, or more compared to the control sample.

50. The method of any one of claims 25-49, wherein the method further comprises measuring an expression level of an internal control and wherein the expression level of the internal control is unaffected by the caveolin-1 peptide or derivative thereof.

51. The method of any one of claims 25-50, wherein steps (a)-(c) of the method are repeated one or more times.

52. A method of predicting or determining the efficacy of a therapeutically active agent, the method comprising:
(a) treating a cell of a subject with interstitial lung disease with the therapeutically active agent;
(b) measuring an expression level of a biomarker in the cell of the subject;
wherein the biomarker is MYDGF, soluble RAGE, pSMAD2/3, PDGFRβ, pPDGFRβ, LGALS7, IL-11, MMP-2, CXCL7, sCD163, p-mTOR, PROF1, CALM2, CALR, PPIA, or EIF5A1; and
(c) comparing the expression level of the biomarker to a control sample.

53. The method of claim 52, wherein the control sample is a cell obtained from the subject with interstitial lung disease prior to treatment with the therapeutically active agent.

54. The method of claim 52, wherein the control sample is a cell obtained from a subject or a population of subjects with interstitial lung disease.

55. The method of claim 52, wherein the control sample is a cell obtained from a healthy subject.

56. The method of any one of claims 52-55, wherein the cell is a fibroblast, a type I alveolar epithelial cell, a type II alveolar epithelial cell, a basal-like cell, a Clara cell, a ciliated cell, a club cell, a goblet cell, a neuroendocrine cell, an endothelial cell, a bialveolar stem cell, a macrophage, an alveolar macrophage, an ionocyte, a pericyte, a mesothelial cell, a mesenchymal cell, a neuroendocrine cell, a myofibroblast, a B-cell, a plasma cell, an innate lymphoid cell, a T-cell, a monocyte, an NK cell, a dendritic cell, or a PBMC.

57. The method of claim 56, wherein the cell is a fibroblast.

58. The method of claim 56, wherein the cell is a basal-like cell.

59. The method of any one of claims 52-58, wherein the cell is treated with the therapeutically active agent ex vivo or in vitro.

60. The method of claim 59, wherein the cell is obtained from the subject with interstitial lung disease.

61. The method of any one of claims 52-60, wherein the therapeutically active agent is a small molecule.

62. The method of any one of claims 52-60, wherein the therapeutically active agent is a biologic.

63. The method of claim 62, wherein the biologic is a caveolin-1 peptide or derivative thereof.

64. The method of claim 63, wherein the caveolin-1 peptide or derivative thereof is FTTFTVT (SEQ ID NO: 3).

65. The method of claim 63, wherein the caveolin-1 peptide or derivative thereof is Ac-aaEGKASFTTFTVTKGSaa-NH2 (SEQ ID NO: 4).

66. The method of any one of claims 52-65, wherein the subject is a human.

67. The method of any one of claims 52-66, wherein the interstitial lung disease is idiopathic pulmonary fibrosis, lymphangioleiomyomatosis, nonspecific interstitial pneumonia, idiopathic interstitial pneumonia, cryptogenic organizing pneumonia, acute interstitial pneumonia, respiratory bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease.

68. The method of claim 67, wherein the interstitial lung disease is idiopathic pulmonary fibrosis.

69. The method of any one of claims 52-68, wherein the expression level of the biomarker is measured by 2-D gel electrophoresis, Western blot, mass spectrometry, flow cytometry, quantitative RT-PCR, ELISA, and/or a lateral flow immunoassay.

70. The method of any one of claims 52-69, wherein the increased expression level of MYDGF, PROF1, CALM2, CALR, PPIA, IF5A1, LGALS7, soluble RAGE, or EIF5A1 indicates a favorable response to the therapeutically active agent.

71. The method of any one of claims 52-69, wherein the decreased expression level of PDGFRβ, pPDGFRβ, p-mTOR, pSMAD2/3, IL-11, MMP-2, CXCL7, sCD163, or CALR indicates a favorable response to the therapeutically active agent.

72. The method of any one of claims 52-69, wherein the expression level of MYDGF is increased compared to the control sample.

73. The method of claim 72, wherein the expression level of MYDGF is increased by about 1.5-fold, about 2-fold, about 3-fold, about 4-fold, about 5-fold, or more compared to the control sample.

74. The method of any one of claims 52-69, wherein the expression level of soluble RAGE is increased compared to the control sample.

75. The method of claim 74, wherein the expression level of soluble RAGE is increased by about 1.5-fold, about 2-fold, about 3-fold, about 4-fold, about 5-fold, or more compared to the control sample.

76. The method of any one of claims 52-69, wherein the expression level of pSMAD2/3 is decreased compared to the control sample.

77. The method of claim 76, wherein the expression level of pSMAD2/3 is decreased by about 10%, about 25%, about 50%, about 75%, about 90%, or more compared to the control sample.

78. The method of any one of claims 52-69, wherein the expression level of PDGFRβ and/or pPDGFRβ is decreased compared to the control sample.

79. The method of claim 78, wherein the expression level of PDGFRβ and/or pPDGFRβ is decreased by about 10%, about 25%, about 50%, about 75%, about 90%, or more compared to the control sample.

80. The method of any one of claims 52-79, wherein the method further comprises measuring an expression level of an internal control and wherein the expression level of the internal control is unaffected by the therapeutically active agent.

81. The method of any one of claims 52-80, wherein steps (a)-(c) of the method are repeated one or more times.

82. A method of predicting or determining the efficacy of a caveolin-1 peptide or derivative thereof, the method comprising:
(a) treating a biological sample from a subject with fibrosis with the caveolin-1 peptide or derivative thereof;
(b) measuring an expression level of a biomarker in the biological sample from the subject;
wherein the biomarker is MYDGF, soluble RAGE, pSMAD2/3, PDGFRβ, pPDGFRβ, LGALS7, IL-11, MMP-2, CXCL7, sCD163, p-mTOR, PROF1, CALM2, CALR, PPIA, or IF5A1; and
(c) comparing the expression level of the biomarker to a control sample.

83. The method of claim 82, wherein the control sample is a biological sample obtained from the subject with fibrosis prior to treatment with the caveolin-1 peptide or derivative thereof.

84. The method of claim 82, wherein the control sample is a biological sample obtained from a subject or a population of subjects with fibrosis.

85. The method of claim 82, wherein the control sample is a biological sample obtained from a healthy subject.

86. The method of any one of claims 82-85, wherein the biological sample is bronchoalveolar lavage fluid (BALF), lung tissue, fibroblasts, type I alveolar epithelial cells, type II alveolar epithelial cells, basal cells, Clara cells, ciliated cells, club cells, goblet cells, neuroendocrine cells, endothelial cells, bialveolar stem cells, macrophages, alveolar macrophages, ionocytes, pericytes, mesothelial cells, mesenchymal cells, neuroendocrine cells, myofibroblasts, B-cells, plasma cells, innate lymphoid cells, T-cells, monocytes, NK cells, dendritic cells, or PBMCs.

87. The method of claim 86, wherein the biological sample is fibroblasts.

88. The method of claim 86, wherein the biological sample is basal-like cells.

89. The method of claim 86, wherein the biological sample is lung tissue.

90. The method of any one of claims 82-85, wherein biological sample is treated with the caveolin-1 peptide or derivative thereof ex vivo or in vitro.

91. The method of claim 90, wherein the biological sample is obtained from the subject with fibrosis.

92. The method of any one of claims 82-91, wherein the caveolin-1 peptide or derivative thereof is FTTFTVT (SEQ ID NO: 3).

93. The method of any one of claims 82-91, wherein the caveolin-1 peptide or derivative thereof is Ac-aaEGKASFTTFTVTKGSaa-NH2 (SEQ ID NO: 4).

94. The method of any one of claims 82-93, wherein the subject is a human.

95. The method of any one of claims 82-94, wherein the fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

96. The method of claim 95, wherein the interstitial lung disease is idiopathic pulmonary fibrosis, lymphangioleiomyomatosis, nonspecific interstitial pneumonia, idiopathic interstitial pneumonia, cryptogenic organizing pneumonia, acute interstitial pneumonia, respiratory bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease.

97. The method of claim 96, wherein the interstitial lung disease is idiopathic pulmonary fibrosis.

98. The method of any one of claims 82-97, wherein the expression level of the biomarker is measured by 2-D gel electrophoresis, Western blot, mass spectrometry, flow cytometry, quantitative RT-PCR, ELISA, and/or a lateral flow immunoassay.

99. The method of any one of claims 82-98, wherein the increased expression level of MYDGF, PROF1, CALM2, CALR, PPIA, IF5A1, LGALS7, soluble RAGE or EIF5A1 indicates a favorable response to the caveolin-1 peptide or derivative thereof.

100. The method of any one of claims 82-98, wherein the decreased expression level of PDGFRβ, pPDGFRβ, p-mTOR, CALR, IL-11, MMP-2, CXCL7, sCD163, or pSMAD2/3 indicates a favorable response to the caveolin-1 peptide or derivative thereof.

101. The method of any one of claims 82-98, wherein the expression level of MYDGF is increased compared to the control sample.

102. The method of claim 101, wherein the expression level of MYDGF is increased by about 1.5-fold, about 2-fold, about 3-fold, about 4-fold, about 5-fold, or more compared to the control sample.

103. The method of any one of claims 82-98, wherein the expression level of soluble RAGE is increased compared to the control sample.

104. The method of claim 103, wherein the expression level of soluble RAGE is increased by about 1.5-fold, about 2-fold, about 3-fold, about 4-fold, about 5-fold, or more compared to the control sample.

105. The method of any one of claims 82-98, wherein the expression level of pSMAD2/3 is decreased compared to the control sample.

106. The method of claim 105, wherein the expression level of pSMAD2/3 is decreased by about 10%, about 25%, about 50%, about 75%, about 90%, or more compared to the control sample.

107. The method of any one of claims 82-98, wherein the expression level of PDGFRβ and/or pPDGFRβ is decreased compared to the control sample.

108. The method of claim 107, wherein the expression level of PDGFRβ and/or pPDGFRβ is decreased by about 10%, about 25%, about 50%, about 75%, about 90%, or more compared to the control sample.

109. The method of any one of claims 82-108, wherein the method further comprises measuring an expression level of an internal control and wherein the expression level of the internal control is unaffected by the caveolin-1 peptide or derivative thereof.

110. The method of any one of claims 82-109, wherein steps (a)-(c) of the method are repeated one or more times.

111. A method of predicting or determining the efficacy of a caveolin-1 peptide or derivative thereof in a subject with fibrosis, the method comprising:
(a) obtaining a first biological sample from the subject prior to treatment with the caveolin-1 peptide or derivative thereof;
(b) administering the caveolin-1 peptide or derivative thereof to the subject;
(c) obtaining a second biological sample from the subject following treatment with the caveolin-1 peptide or derivative thereof;
(d) measuring an expression level of a biomarker in the first biological sample and the second biological sample;
wherein the biomarker is MYDGF, soluble RAGE, pSMAD2/3, PDGFRβ, pPDGFRβ, LGALS7, IL-11, MMP-2, CXCL7, sCD163, p-mTOR, PROF1, CALM2, CALR, PPIA, or IF5A1; and
(e) comparing the expression level of the biomarker in the first biological sample to the expression level of the biomarker in the second biological sample.

112. The method of claim 111, wherein step (a) of the method further comprises determining the expression level of the biomarker in the first biological sample compared to a control sample prior to treatment with the caveolin-1 peptide or derivative thereof.

113. The method of claim 112, wherein the control sample is a biological sample obtained from a subject or a population of subjects with fibrosis.

114. The method of claim 112 or 113, wherein the expression level of the biomarker in the first biological sample of step (a) is used to determine an optimal dose of the caveolin-1 peptide or derivative thereof administered in step (b).

115. The method of any one of claims 111-114, wherein the biological sample is serum, plasma, BALF, lung tissue, fibroblasts, type I alveolar epithelial cells, type II alveolar epithelial cells, basal cells, Clara cells, ciliated cells, club cells, goblet cells, neuroendocrine cells, endothelial cells, bialveolar stem cells, macrophages, alveolar macrophages, ionocytes, pericytes, mesothelial cells, mesenchymal cells, neuroendocrine cells, myofibroblasts, B-cells, plasma cells, innate lymphoid cells, T-cells, monocytes, NK cells, dendritic cells, or PBMCs.

116. The method of claim 115, wherein the biological sample is serum.

117. The method of claim 115, wherein the biological sample is plasma.

118. The method of claim 115, wherein the biological sample is fibroblasts, basal-like cells, or PBMCs.

119. The method of any one of claims 111-118, wherein the caveolin-1 peptide or derivative thereof is FTTFTVT (SEQ ID NO: 3).

120. The method of any one of claims 111-118, wherein the caveolin-1 peptide or derivative thereof is Ac-aaEGKASFTTFTVTKGSaa-NH2 (SEQ ID NO: 4).

121. The method of any one of claims 1-24 and 87-96, wherein the subject is a human.

122. The method of any one of claims 111-121, wherein the fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

123. The method of claim 122, wherein the interstitial lung disease is idiopathic pulmonary fibrosis, familial pulmonary fibrosis, idiopathic nonspecific interstitial pneumonia, conventional interstitial pneumonia, cryptogenic organizing pneumonia, or sarcoidosis.

124. The method of claim 123, wherein the interstitial lung disease is idiopathic pulmonary fibrosis.

125. The method of any one of claims 111-124, wherein the expression level of the biomarker in the first and second biological sample is measured by 2-D gel electrophoresis, Western blot, mass spectrometry, flow cytometry, quantitative RT-PCR, ELISA, and/or a lateral flow immunoassay.

126. The method of any one of claims 111-125, wherein the increased expression level of MYDGF, PROF1, CALM2, CALR, PPIA, IF5A1, LGALS7, or soluble RAGE indicates a favorable response to the caveolin-1 peptide or derivative thereof.

127. The method of any one of claims 111-125, wherein the decreased expression level of PDGFRβ, pPDGFRβ, p-mTOR, CALR, IL-11, MMP-2, CXCL7, sCD163, or pSMAD2/3 indicates a favorable response to the caveolin-1 peptide or derivative thereof.

128. The method of any one of claims 1-24 and 111-127, wherein the caveolin-1 peptide or derivative thereof is administered to the subject at a dose of about 0.01 mg/kg to about 250 mg/kg.

129. The method of claim 128, wherein the caveolin-1 peptide or derivative thereof is administered to the subject at a dose of about 0.05 mg/kg to about 50 mg/kg.

130. The method of any one of claims 1-24 and 111-129, wherein the caveolin-1 peptide or derivative thereof is administered to the subject through inhalation, intravenously, subcutaneously, orally, intraperitoneally, sublingually, buccally, or intramuscularly.

131. The method of any one of claims 1-24 and 111-130, wherein the method comprises administering the caveolin-1 peptide or derivative thereof to the subject once per day, once per week, twice per week, three times per week, five times per week, once every two weeks, or once per month.

132. The method of any one of claims 1-24 and 111-131, wherein the second biological sample is obtained from the subject one hour, three hours, six hours, twelve hours, one day, two days, three days, four days, five days, one week, two weeks, three weeks, one month, six months, or one year following administration of the caveolin-1 peptide or derivative thereof.

133. The method of any one of claims 1-24 and 87-108, wherein the method further comprises obtaining one or more additional biological samples following administration of the caveolin-1 peptide or derivative thereof.

134. The method of claim 133, wherein the expression level of the biomarker in the one or more additional biological samples is increased compared to the expression level of the biomarker in the first biological sample; and wherein the increased expression level of the biomarker in the one or more additional biological samples indicates a favorable response to the caveolin-1 peptide or derivative thereof.

135. The method of claim 133, wherein the expression level of the biomarker in the one or more additional biological samples is decreased compared to the expression level of the biomarker in the first biological sample; and wherein the decreased expression level of the biomarker in the one or more additional biological samples indicates a favorable response to the caveolin-1 peptide or derivative thereof.

136. The method of any one of claims 111-132, wherein the expression level of MYDGF in the second biological sample is increased compared to the first biological sample.

137. The method of claim 136, wherein the expression level of MYDGF in the second biological sample is increased by about 1.5-fold, about 2-fold, about 3-fold, about 4-fold, about 5-fold, or more compared to the first biological sample.

138. The method of any one of claims 111-132, wherein the expression level of soluble RAGE in the second biological sample is increased compared to the first biological sample.

139. The method of claim 138, wherein the expression level of soluble RAGE in the second biological sample is increased by about 1.5-fold, about 2-fold, about 3-fold, about 4-fold, about 5-fold, or more compared to the first biological sample.

140. The method of any one of claims 111-132, wherein the expression level of pSMAD2/3 is decreased compared to the control sample.

141. The method of claim 140, wherein the expression level of pSMAD2/3 is decreased by about 10%, about 25%, about 50%, about 75%, about 90%, or more compared to the control sample.

142. The method of any one of claims 111-132, wherein the expression level of PDGFRβ and/or pPDGFRβ in the second biological sample is decreased in the second biological sample compared to the first biological sample.

143. The method of claim 142, wherein the expression level of PDGFRβ and/or pPDGFRβ in the second biological sample is decreased by about 10%, about 25%, about 50%, about 75%, about 90%, or more compared to the first biological sample.

144. The method of any one of claims 111-132, wherein the expression level of the biomarker in the second biological sample is used to determine an optimal dose of the caveolin-1 peptide or derivative thereof.
